SEQUENCE LISTING (268 sequences)

SEQ ID NO: 1 - protein, 14 aa, synthetic construct
GRCTFSIPPICFPD

SEQ ID NO: 2 - protein, 14 aa, synthetic construct
GRCTYSIPPICFPD

SEQ ID NO: 3 - protein, 13 aa, synthetic construct
GRCTYSIPPICFP

SEQ ID NO: 4 - protein, 12 aa, synthetic construct
GRCTYSIPPICF

SEQ ID NO: 5 - protein, 11 aa, synthetic construct
RCTYSIPPICF

SEQ ID NO: 6 - protein, 14 aa, synthetic construct
GTCTFSIPPICNPN

SEQ ID NO: 7 - protein, 14 aa, synthetic construct
GTCTYSIPPICNPN

SEQ ID NO: 8 - protein, 13 aa, synthetic construct
GTCTYSIPPICNP

SEQ ID NO: 9 - protein, 12 aa, synthetic construct
GTCTYSIPPICN

SEQ ID NO: 10 - protein, 11 aa, synthetic construct
TCTYSIPPICN

SEQ ID NO: 11 - protein, 11 aa, synthetic construct
KCTYSIPPICF

SEQ ID NO: 12 - protein, 15 aa, synthetic construct
CGRATYSIPPICFPD

SEQ ID NO: 13 - protein, 14 aa, synthetic construct
CGRATYSIPPICFP

SEQ ID NO: 14 - protein, 13 aa, synthetic construct
CGRATYSIPPICF

SEQ ID NO: 15 - protein, 13 aa, synthetic construct
  MOD_RES 4: note = L-alpha-Aminobutyric acid
CGRXTYSIPPICF

SEQ ID NO: 16 - protein, 13 aa, synthetic construct
CKRATYSIPPICF

SEQ ID NO: 17 - protein, 13 aa, synthetic construct
CERATYSIPPICF

SEQ ID NO: 18 - protein, 13 aa, synthetic construct
CGRATFSIPPICF

SEQ ID NO: 19 - protein, 12 aa, synthetic construct
FCTYSIPPQCYG

SEQ ID NO: 20 - protein, 12 aa, synthetic construct
FCTFSIPPQCYG

SEQ ID NO: 21 - protein, 12 aa, synthetic construct
SCTFSIPPQCYG

SEQ ID NO: 22 - protein, 11 aa, synthetic construct
ACTYSIPAKCF

SEQ ID NO: 23 - protein, 14 aa, synthetic construct
GTCTFSIPPICNPN

SEQ ID NO: 24 - protein, 12 aa, synthetic construct
GTCTFSIPPICN

SEQ ID NO: 25 - protein, 11 aa, synthetic construct
FCTYSIPPQCY

SEQ ID NO: 26 - protein, 12 aa, synthetic construct
FCTYSIPPQCYA

SEQ ID NO: 27 - protein, 11 aa, synthetic construct
FCTYSIPPQCR

SEQ ID NO: 28 - protein, 11 aa, synthetic construct
PCTYSIPPQCY

SEQ ID NO: 29 - protein, 21 aa, synthetic construct
GIVEQCCTSICSLYQLENYCN

SEQ ID NO: 30 - protein, 21 aa, synthetic construct
GIVEQCCTSICSLYQLENYCA

SEQ ID NO: 31 - protein, 22 aa, synthetic construct
GIVEQCCTSICSLYQLENYCNG

SEQ ID NO: 32 - protein, 22 aa, synthetic construct
GIVEQCCTSICSLYQLENYCAG

SEQ ID NO: 33 - protein, 21 aa, synthetic construct
GIVEQCCTSICSLYQLENYCG

SEQ ID NO: 34 - protein, 30 aa, synthetic construct
FVNQHLCGSHLVEALYLVCGERGFFYTPKT

SEQ ID NO: 35 - protein, 30 aa, synthetic construct
FVNQHLCGSHLVEALYLVCGERGFFYTDKT

SEQ ID NO: 36 - protein, 30 aa, synthetic construct
FVNQHLCGSHLVEALYLVCGERGFFYTKPT

SEQ ID NO: 37 - protein, 30 aa, synthetic construct
FVNQHLCGSHLVEALYLVCGEEGFFYTPKT

SEQ ID NO: 38 - protein, 30 aa, synthetic construct
FVNQHLCGSHLVEALYLVCGEDGFFYTKPT

SEQ ID NO: 39 - protein, 30 aa, synthetic construct
FVNQHLCGSHLVEALYLVCGEEGFFYTKPT

SEQ ID NO: 40 - protein, 32 aa, synthetic construct
FVNQHLCGSHLVEALYLVCGERGFFYTKPTRR

SEQ ID NO: 41 - protein, 44 aa, synthetic construct
GRCTYSIPPICFGGFVNQHLCGSHLVEALYLVCGERGFFYTPKT

SEQ ID NO: 42 - protein, 43 aa, synthetic construct
FCTYSIPPQCYGGFVNQHLCGSHLVEALYLVCGERGFFYTPKT

SEQ ID NO: 43 - protein, 44 aa, synthetic construct
GRCTYSIPPICFGGFVNQHLCGSHLVEALYLVCGERGFFYTDKT

SEQ ID NO: 44 - protein, 43 aa, synthetic construct
FCTYSIPPQCYGGFVNQHLCGSHLVEALYLVCGERGFFYTDKT

SEQ ID NO: 45 - protein, 44 aa, synthetic construct
GRCTYSIPPICFGGFVNQHLCGSHLVEALYLVCGERGFFYTKPT

SEQ ID NO: 46 - protein, 43 aa, synthetic construct
FCTYSIPPQCYGGFVNQHLCGSHLVEALYLVCGERGFFYTKPT

SEQ ID NO: 47 - protein, 46 aa, synthetic construct
GRCTYSIPPICFGGFVNQHLCGSHLVEALYLVCGERGFFYTKPTRR

SEQ ID NO: 48 - protein, 45 aa, synthetic construct
FCTYSIPPQCYGGFVNQHLCGSHLVEALYLVCGERGFFYTKPTRR

SEQ ID NO: 49 - protein, 44 aa, synthetic construct
GRCTYSIPPICFGGFVNQHLCGSHLVEALYLVCGEEGFFYTKPT

SEQ ID NO: 50 - protein, 43 aa, synthetic construct
FCTYSIPPQCYGGFVNQHLCGSHLVEALYLVCGEEGFFYTKPT

SEQ ID NO: 51 - protein, 43 aa, synthetic construct
FVNQHLCGSHLVEALYLVCGERGFFYTPKTGRCTYSIPPICFG

SEQ ID NO: 52 - protein, 43 aa, synthetic construct
FVNQHLCGSHLVEALYLVCGERGFFYTPKTGFCTYSIPPQCYG

SEQ ID NO: 53 - protein, 43 aa, synthetic construct
FVNQHLCGSHLVEALYLVCGERGFFYTDKTGRCTYSIPPICFG

SEQ ID NO: 54 - protein, 43 aa, synthetic construct
FVNQHLCGSHLVEALYLVCGERGFFYTDKTGFCTYSIPPQCYG

SEQ ID NO: 55 - protein, 43 aa, synthetic construct
FVNQHLCGSHLVEALYLVCGERGFFYTKPTGRCTYSIPPICFG

SEQ ID NO: 56 - protein, 43 aa, synthetic construct
FVNQHLCGSHLVEALYLVCGERGFFYTKPTGFCTYSIPPQCYG

SEQ ID NO: 57 - protein, 43 aa, synthetic construct
FVNQHLCGSHLVEALYLVCGEEGFFYTKPTGRCTYSIPPICFG

SEQ ID NO: 58 - protein, 43 aa, synthetic construct
FVNQHLCGSHLVEALYLVCGEEGFFYTKPTGFCTYSIPPQCYG

SEQ ID NO: 59 - protein, 35 aa, synthetic construct
GRCTYSIPPICFGGIVEQCCTSICSLYQLENYCNG

SEQ ID NO: 60 - protein, 35 aa, synthetic construct
GFCTYSIPPQCYGGIVEQCCTSICSLYQLENYCNG

SEQ ID NO: 61 - protein, 34 aa, synthetic construct
GRCTYSIPPICFGGIVEQCCTSICSLYQLENYCG

SEQ ID NO: 62 - protein, 34 aa, synthetic construct
GFCTYSIPPQCYGGIVEQCCTSICSLYQLENYCG

SEQ ID NO: 63 - protein, 35 aa, synthetic construct
GIVEQCCTSICSLYQLENYCNGGRCTYSIPPICFG

SEQ ID NO: 64 - protein, 35 aa, synthetic construct
GIVEQCCTSICSLYQLENYCNGGFCTYSIPPQCYG

SEQ ID NO: 65 - protein, 28 aa, synthetic construct
GIVEQCRCTYSIPPICFGSLYQLENYCN

SEQ ID NO: 66 - protein, 28 aa, synthetic construct
GIVEQCFCTYSIPPQCYGSLYQLENYCN

SEQ ID NO: 67 - protein, 28 aa, synthetic construct
GIVEQCRCTYSIPPICFGSLYQLENYCG

SEQ ID NO: 68 - protein, 28 aa, synthetic construct
GIVEQCFCTYSIPPQCYGSLYQLENYCG

SEQ ID NO: 69 - protein, 6 aa, synthetic construct
ENLYFQ

SEQ ID NO: 70 - protein, 18 aa, synthetic construct
RREAEDGSGGSGENLYFQ

SEQ ID NO: 71 - protein, 35 aa, synthetic construct
RREAEDLQVGQVELGGGPGAGSLQPLALGENLYFQ

SEQ ID NO: 72 - protein, 26 aa, synthetic construct
GSRHWGGGPGAGSLQPLALGENLYFQ

SEQ ID NO: 73 - protein, 18 aa, synthetic construct
GSRHWGGSGGSGENLYFQ

SEQ ID NO: 74 - protein, 30 aa, synthetic construct
FVQQHLCGSHLVEALYLVCGERGFFYTPKT

SEQ ID NO: 75 - protein, 30 aa, synthetic construct
FVNQHLCGSHLVEALYLVCGERGFFYTPKQ

SEQ ID NO: 76 - protein, 30 aa, synthetic construct
FVNQHLCGSHLVEALYLVCGERGFFYTPKE

SEQ ID NO: 77 - protein, 30 aa, synthetic construct
FVQQHLCGSHLVEALYLVCGERGFFYTPKE

SEQ ID NO: 78 - protein, 30 aa, synthetic construct
FVNQHLCGSHLVEALYLVCGERGFFYTDPT

SEQ ID NO: 79 - protein, 30 aa, synthetic construct
FVNQHLCGSHLVQALYLVCGERGFFYTPKT

SEQ ID NO: 80 - protein, 30 aa, synthetic construct
FVNQHLCGSHLVEALALVCGERGFFYTPKT

SEQ ID NO: 81 - protein, 30 aa, synthetic construct; identical to SEQ ID NO: 37

SEQ ID NO: 82 - protein, 30 aa, synthetic construct
FVNQHLCGSHLVEALYLVCGEEGFFYTLPT

SEQ ID NO: 83 - protein, 30 aa, synthetic construct
FVNQHLCGSHLVEALYLVCGERGFFYTVPT

SEQ ID NO: 84 - protein, 30 aa, synthetic construct
FVNQHLCGSHLVEALYLVCGERGFFYTAPT

SEQ ID NO: 85 - protein, 21 aa, synthetic construct
GIVEQCCTSICSLEQLENYCN

SEQ ID NO: 86 - protein, 21 aa, synthetic construct
GIVEQCCTSICSLYQLENYCS

SEQ ID NO: 87 - protein, 21 aa, synthetic construct
GIVEQCCHSICSLYQLENYCN

SEQ ID NO: 88 - protein, 21 aa, synthetic construct
GIVEQCCWSICSLYQLENYCN

SEQ ID NO: 89 - protein, 21 aa, synthetic construct
GIVEQCCMSICSLYQLENYCN

SEQ ID NO: 90 - DNA, 1729 bp, synthetic construct
  source 1..1729: mol_type = other DNA
tgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatccaacccttaatataacttcgtataatgtatgctatacgaagttattaggtctcgaacagatccgccaggcgtgtatatatagcgtggatggccaggcaactttagtgctgacacatacaggcatatatatatgtgtgcgacgacacatgatcatatcgcatgcatgtgctctgtatgtatataaaactcttgttttcttcttttctctaaatattctttccttatacattaggacctttgcagcataaattactatacttctatagacacacaaacacaaatacacacactaaattaataatgagccatattcaacgggaaacgtcttgctctaggccgcgattaaattccaacatggatgctgatttatatgggtataaatgggctcgtgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgtgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcaacccttaatataacttcgtataatgtatgctatacgaagttattaggtgcatgc

SEQ ID NO: 91 - DNA, 46 bp, synthetic construct
  source 1..46: mol_type = other DNA
cacttcattgataccattattgtacaacttgagcaagttgtcgatc

SEQ ID NO: 92 - DNA, 50 bp, synthetic construct
  source 1..50: mol_type = other DNA
ctgtgcggtatttcacaccgcatagcacctaataacttcgtatagcatac

SEQ ID NO: 93 - DNA, 1578 bp, synthetic construct
  source 1..1578: mol_type = other DNA
atgaaactctcaactattgttacagctgttttgtttacagcttcttccgttttagccgccccagttaataccactaccgaaaatgaaacagctcaaattcctgccgaagctatcattggctacttggatttggagggtgattctgacgttgctgttttgccatttgcaaacagcaccaacactggtttactgttcataaatactactatttcaaaccttgctgccaaggaagaaggatccctcgagaagagaactagtgtgtatagtcaataatagccggagtatttccagcatatatgtatctgtagtgagggttggtggtctgacgaacatccagcaaggtgttccacctgaaatttttcaccttggagggtaatgtgatgacgccatttcctgtgcaaatgcttttcgttttgaacagtgcaacttttgtatcaaatcttcatctacttgatgccatctcaacaaatccctcatttactagcgtgtgaaggaatcaagattttccactgataagccaatttgtcggaaatcccccgcgcgggagttggcgttcagtacgaagcttgatccaataaaaaaacgttatagaaagaaattggactacgatatgctccaatccaaattgtcaaaattgaccaccgaaaaagaacaattggaatttgacaagaggaacaactcactagattctcaaacggagcgtcacctagagtcagtttccaagtcaattacagaaagtttggaaacagaagaggagtatctacaattgaattgcaaacttaaagtcgagctgtcccaattcatgtcgctaaggctttcttacttggaccccatttttgaaagtttcattaaagttcagtcaaaaattttcatggacatttatgacacattaaagagcggactaccttatgttgattctctatccaaagaggattatcagtccaaaatcttggactttagaatagataacattctgtcgaaaatggaagcgctgaaccttcaaccttacattgatgattagagcaatgatataaacaacaattgagtgacaggtctactttgttctcaaaaggccataaccatctgtttgcatctcttatcaccacaccatcctcctcatctggccttcaattgtggggaacaactagcatcccaacaccagactaactccacccagatgaaaccagttgtcgcttaccagtcaatgaatgttgagctaacgttccttgaaactcgaatgatcccagccttgctgcgtatcatccctccgctattccgccgcttgctccaaccatgtttccgcctttttcgaacaagttcaaatacctatctttggcaggacttttcctcctgccttttttagcctcaggtctcggttagcctctaggcaaattctggtcttcatacctatatcaacttttcatcagatagcctttgggttcaaaaaagaactaaagcaggatgcctgatatataaatcccagatgatctgcttttgaaactattttcagtatcttgattcgtttacttacaaacaactattgttgattttatctggagaataatcgaacaaagatcc

SEQ ID NO: 94 - DNA, 53 bp, synthetic construct
  source 1..53: mol_type = other DNA
aaaacaactaattattcgaaggatcaaaatgaaactctcaactattgttacag

SEQ ID NO: 95 - DNA, 53 bp, synthetic construct
  source 1..53: mol_type = other DNA
tgaaggaaatctcatcgtttggatctttgttcgattattctccagataaaatc

SEQ ID NO: 96 - DNA, 58 bp, synthetic construct
  source 1..58: mol_type = other DNA
gagaaaataccgcatcaggcgctcttccgctaagcttcaacgatgccaaaagggtata

SEQ ID NO: 97 - DNA, 54 bp, synthetic construct
  source 1..54: mol_type = other DNA
gcgcagcgagtcagtgagcgaggaagcagatcttgagataaatttcacgtttaa

SEQ ID NO: 98 - DNA, 885 bp, Pichia pastoris
  source 1..885: mol_type = unassigned DNA
aagcttcaacgatgccaaaagggtataataagcgtcatttgcagcattgtgaagaaaactatgtggcaagccaagcctgcgaagaatgtattttaagtttgactttgatgtattcacttgattaagccataattctcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccagacgataataccaatctaaactctttaaacgttaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatct

SEQ ID NO: 99 - DNA, 48 bp, synthetic construct
  source 1..48: mol_type = other DNA
gaggctgaagcttacgtagaattcgatagttcacttcttcctgttaag

SEQ ID NO: 100 - DNA, 49 bp, synthetic construct
  source 1..49: mol_type = other DNA
ctaaggcgaattaattcgcggccgcctcacgatcttccggaagatggag

SEQ ID NO: 101 - DNA, 2199 bp, synthetic construct
  source 1..2199: mol_type = other DNA
gatagttcacttcttcctgttaaggaggcagaagacaagttgtctattaacgaccctttgttcgaaagacaatggcatctagttaatccttcattcccaggttcagacattaacgtattggacttatggtataacaacataactggtgctggtgttgttgcagcaatagtagacgacggtttggattatgagaacgaggatctaaaagacaacttctgtgcagagggaagctgggactttaatgataacactaacttgccaaagcctcgtttgagcgacgattatcacggaactaggtgcgctggagagattgcagctaagaagggaaataacttctgtggagttggtgttggatataatgcaaagattagtggtattaggattcttagtggagacattacaactgaggacgaggcagctagtcttatatacggattggatgttaatgacatttacagttgtagttggggaccagcagacgatggtaggcaccttcagggtccatcagatttagttaagaaagcacttgttaagggagtaacagaaggtagggacagtaagggtgctatatatgtattcgcttcaggtaacggaggtacaagaggagacaactgtaactatgatggttacacaaacagtatttacagcataacaataggtgcaatagaccataaggacttgcacccaccatacagtgagggatgcagtgctgttatggctgttacttacagcagtggaagtggtgagtacatacacagctcagacattaatggtcgttgttcaaactctcatggaggtactagcgctgctgcacctcttgcagctggagtatatacacttcttttggaggctaatcctaatttgacatggcgtgatgttcaataccttagtattcttagcgctgttggtcttgagaaaaatgcagatggtgactggcgtgactctgctatgggtaaaaagtatagccacagatacggtttcggaaagattgacgctcacaaacttatagagatgagtaaaacttgggaaaacgtaaatgctcagacttggttctatttacctactctttacgtaagtcaaagtactaatagtactgaggaaactcttgagagtgttattactattagcgagaagtcattgcaggacgcaaattttaaacgtatagaacatgttactgttacagttgacatagacactgagatacgtggtacaactacagttgaccttattagtcctgctggtattataagtaatttgggtgtagttcgtcctcgtgacgtaagtagcgaaggttttaaggattggacttttatgagcgttgctcactggggagaaaatggtgttggagactggaagattaaagtaaaaactactgagaacggtcatagaatagattttcattcatggcgtttaaaattgttcggtgagagtatagacagtagcaagactgagacatttgttttcggtaatgacaaggaagaagtagagccagcagcaactgagtcaactgttagtcagtacagcgcttcaagcacaagcataagtatttctgcaacaagcactagcagcattagtataggagttgagacttctgctatacctcagactacaacagcttcaactgacccagacagcgaccctaatacaccaggaggtagcgcaaataatttgagtaatgaatcaaacggaacaaaccacagtaatcacacatcaatcgacggtggatctggtcctgattctgatccaaacactcctaaaaaactttcctctccaaggcaagccatgcattattttttaacaatatttttgattggtgctacatttttggtgttatacttcatgttttttatgaaatcaaggagaagaatcagaaggtcaagagctgaaacttatgaatttgatattattgatacagactctgagtacgattctactttggacaatggaacttctggaattactgagcctgaagaggttgaggacttcgattttgatttgtctgatgaagatcatttggcatctttgtcttcatcagaaaacggtgatgctgaacatacaattgattctgtattaacaaacgaaaatccattttctgaccctataaagcaaaagttcccaaatgacgccaacgcagaatctgcttccaataaattacaagaattacagcctgatgttcctccatcttccggaagatcgtga

SEQ ID NO: 102 - DNA, 1457 bp, synthetic construct
  source 1..1457: mol_type = other DNA
ctgttgtagttttaatatagtttgagtatgagatggaactcagaacgaaggaattatcaccagtttatatattctgaggaaagggtgtgtcctaaattggacagtcacgatggcaataaacgctcagccaatcagaatgcaggagccataaattgttgtattattgctgcaagatttatgtgggttcacattccactgaatggttttcactgtagaattggtgtcctagttgttatgtttcgagatgttttcaagaaaaactaaaatgcacaaactgaccaataatgtgccgtcgcgcttggtacaaacgtcaggattgccaccacttttttcgcactctggtacaaaagttcgcacttcccactcgtatgtaacgaaaaacagagcagtctatccagaacgagacaaattagcgcgtactgtcccattccataaggtatcataggaaacgagagtcctccccccatcacgtatatataaacacactgatatcccacatccgcttgtcaccaaactaatacatccagttcaagttacctaaacaaatcaaattttcgctgatgagtccgtgaggacgaaacgagtaagctcgtccgaaaaaccagcccagttatgttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgagaccttcgtttgtgcacgcgtgtacgcatgtaacattatactgaaaaccttgcttgagaaggttttgggacgctcgaaggctttaatttgcaagctgggggaagatttattgtctcaaaaggtcaatttcatattttatatgcattcaatacttatttattattaatttagcttgactacgatgcatataattttaattttattttaaattatatatgaggtaagagtataactctaaacctaataaatatataataattatacgcaatagttaaaccatagattaattacaactaatcctttcgtactaagttgtaatcctttattgacatttccctaaagcagatagaaaccatactgtctcacgactattaaacccaactcacgtaaccttttaattgacgaacagtcaaacccttatcagcgtgtgctaccaataggataggttga

SEQ ID NO: 103 - DNA, 51 bp, synthetic construct
  source 1..51: mol_type = other DNA
tgaccacacctctaccggccgctagcctgttgtagttttaatatagtttga

SEQ ID NO: 104 - DNA, 53 bp, synthetic construct
  source 1..53: mol_type = other DNA
tatattaagggttccggatcgcggccgcatctcaacctatcctattggtagca

SEQ ID NO: 105 - DNA, 46 bp, synthetic construct
  source 1..46: mol_type = other DNA
gacgtaccagattacgctcatatggacaagaagtacagcatcggcc

SEQ ID NO: 106 - DNA, 48 bp, synthetic construct
  source 1..48: mol_type = other DNA
tcagtatctacgattcatctcgagtcacaccttcctcttcttcttggg

SEQ ID NO: 107 - DNA, 50 bp, synthetic construct
  source 1..50: mol_type = other DNA
agtgtgcaagtttctgtaaatcgatcgaactgtggggttgcagacagttt

SEQ ID NO: 108 - DNA, 48 bp, synthetic construct
  source 1..48: mol_type = other DNA
ccgatgctgtacttcttgtccatatgtgttttgatagttgttcaattg

SEQ ID NO: 109 - DNA, 852 bp, Pichia pastoris
  source 1..852: mol_type = unassigned DNA
cgaactgtggggttgcagacagtttcaggcgtgtcccgaccaatatggcctactagactctctgaaaaatcacagttttccagtagttccgatcaaattactatcgaaatggtcccataaacggacatttgacatccgttcctgaattatagtcttccaccgtggatcatggtgttcctttttttcccaaagaatatcagcatcccttaactacgttaggtcagtgatgacaatggaccaaattgttgcaaggtttttctttttctttcatcggcacatttcagcctcacatgcgactattatcgatcaatgaaatccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacacat

SEQ ID NO: 110 - protein, 106 aa, synthetic construct
ENLYFQFVNQHLCGSHLVEALYLVCGERGFFYTPKTRREAEDLQVGQVELGGGPGAGSLQPLALGENLYFQGIVEQCCTSICSLYQLENYCNGSRHWGSHHHHHHH

SEQ ID NO: 111 - DNA, 322 bp, synthetic construct
  source 1..322: mol_type = other DNA
gagaatctgtacttccaatttgtgaaccaacacctgtgcggctcacacctggtggaagctctgtacctggtgtgcggagaacgtggattcttctacacaccaaagaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 112 - protein, 106 aa, synthetic construct
ENLYFQFVNQHLCGSHLVEALYLVCGERGFFYTDKTRREAEDLQVGQVELGGGPGAGSLQPLALGENLYFQGIVEQCCTSICSLYQLENYCNGSRHWGSHHHHHHH

SEQ ID NO: 113 - DNA, 322 bp, synthetic construct
  source 1..322: mol_type = other DNA
gagaatctgtacttccaatttgtgaaccaacacctgtgcggctcacacctggtggaagctctgtacctggtgtgcggagaacgtggattcttctacacagataagaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 114 - protein, 106 aa, synthetic construct
ENLYFQFVNQHLCGSHLVEALYLVCGERGFFYTKPTRREAEDLQVGQVELGGGPGAGSLQPLALGENLYFQGIVEQCCTSICSLYQLENYCNGSRHWGSHHHHHHH

SEQ ID NO: 115 - DNA, 322 bp, synthetic construct
  source 1..322: mol_type = other DNA
gagaatctgtacttccaatttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 116 - protein, 106 aa, synthetic construct
ENLYFQFVNQHLCGSHLVEALYLVCGEEGFFYTKPTRREAEDLQVGQVELGGGPGAGSLQPLALGENLYFQGIVEQCCTSICSLYQLENYCNGSRHWGSHHHHHHH

SEQ ID NO: 117 - DNA, 322 bp, synthetic construct
  source 1..322: mol_type = other DNA
gagaatctgtacttccaatttgtgaaccaacacctgtgcggctcacacctggtggaagctctgtacctggtgtgcggagaagagggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 118 - DNA, 46 bp, synthetic construct
  source 1..46: mol_type = other DNA
tgaagccgaggcaggatccgagaatctgtacttccaatttgtgaac

SEQ ID NO: 119 - DNA, 59 bp, synthetic construct
  source 1..59: mol_type = other DNA
attgactatacacactagtgatgatgatggtgatgatgtgatccccaatgtctggatcc

SEQ ID NO: 120 - DNA, 59 bp, synthetic construct
  source 1..59: mol_type = other DNA
gaatagaataagtacacctaccttggaagtacagattctcggatcctgcctcggcttca

SEQ ID NO: 121 - DNA, 59 bp, synthetic construct
  source 1..59: mol_type = other DNA
aggtgtacttattctattcctccaatatgctttggaggttttgtgaaccaacacctgtg

SEQ ID NO: 122 - DNA, 58 bp, synthetic construct
  source 1..58: mol_type = other DNA
aggaatagagtaagtacaaaattggaagtacagattctcggatcctgcctcggcttca

SEQ ID NO: 123 - DNA, 59 bp, synthetic construct
  source 1..59: mol_type = other DNA
atttgtacttactctattcctccacagtgctatggaggttttgtgaaccaacacctgtg

SEQ ID NO: 124 - DNA, 25 bp, synthetic construct
  source 1..25: mol_type = other DNA
cacattgtatgcttccaagattctg

SEQ ID NO: 125 - DNA, 27 bp, synthetic construct
  source 1..27: mol_type = other DNA
gaagatcaaaaaacaactaattattcg

SEQ ID NO: 126 - DNA, 28 bp, synthetic construct
  source 1..28: mol_type = other DNA
gatacatatatgctggaaatactccggc

SEQ ID NO: 127 - DNA, 19 bp, synthetic construct
  source 1..19: mol_type = other DNA
tgaagccgaggcaggatcc

SEQ ID NO: 128 - DNA, 56 bp, synthetic construct
  source 1..56: mol_type = other DNA
aaagcatattggaggaatagaataagtacacctaccggtctttggtgtgtagaaga

SEQ ID NO: 129 - DNA, 55 bp, synthetic construct
  source 1..55: mol_type = other DNA
ttctattcctccaatatgctttggatccagacattggggaggtggtcctggtgca

SEQ ID NO: 130 - DNA, 56 bp, synthetic construct
  source 1..56: mol_type = other DNA
atagcactgtggaggaatagagtaagtacaaaatccggtctttggtgtgtagaaga

SEQ ID NO: 131 - DNA, 53 bp, synthetic construct
  source 1..53: mol_type = other DNA
ctattcctccacagtgctatggatccagacattggggaggtggtcctggtgca

SEQ ID NO: 132 - DNA, 56 bp, synthetic construct
  source 1..56: mol_type = other DNA
aaagcatattggaggaatagaataagtacacctaccggtcttatctgtgtagaaga

SEQ ID NO: 133 - DNA, 56 bp, synthetic construct
  source 1..56: mol_type = other DNA
atagcactgtggaggaatagagtaagtacaaaatccggtcttatctgtgtagaaga

SEQ ID NO: 134 - DNA, 56 bp, synthetic construct
  source 1..56: mol_type = other DNA
aaagcatattggaggaatagaataagtacacctaccggttggctttgtgtagaaga

SEQ ID NO: 135 - DNA, 56 bp, synthetic construct
  source 1..56: mol_type = other DNA
atagcactgtggaggaatagagtaagtacaaaatccggttggctttgtgtagaaga

SEQ ID NO: 136 - DNA, 38 bp, synthetic construct
  source 1..38: mol_type = other DNA
tccccaatgtctggatccaaagcatattggaggaatag

SEQ ID NO: 137 - DNA, 38 bp, synthetic construct
  source 1..38: mol_type = other DNA
tccccaatgtctggatccatagcactgtggaggaatag

SEQ ID NO: 138 - DNA, 58 bp, synthetic construct
  source 1..58: mol_type = other DNA
ggatccagacattggggaggttctggaggtagtggtgagaatctgtacttccaaggca

SEQ ID NO: 139 - DNA, 58 bp, synthetic construct
  source 1..58: mol_type = other DNA
gaggaatagaataagtacacctaccttggaagtacagattctcccccagtgccaatgg

SEQ ID NO: 140 - DNA, 58 bp, synthetic construct
  source 1..58: mol_type = other DNA
gtgtacttattctattcctccaatatgctttggaggcattgtggaacaatgctgtacc

SEQ ID NO: 141 - DNA, 58 bp, synthetic construct
  source 1..58: mol_type = other DNA
gaggaatagagtaagtacaaaatccttggaagtacagattctcccccagtgccaatgg

SEQ ID NO: 142 - DNA, 58 bp, synthetic construct
  source 1..58: mol_type = other DNA
ttgtacttactctattcctccacagtgctatggaggcattgtggaacaatgctgtacc

SEQ ID NO: 143 - DNA, 52 bp, synthetic construct
  source 1..52: mol_type = other DNA
ttggaggaatagaataagtacacctacctccgttgcagtagttctccagctg

SEQ ID NO: 144 - DNA, 52 bp, synthetic construct
  source 1..52: mol_type = other DNA
tacttattctattcctccaatatgctttggatccagacattggggatcacat

SEQ ID NO: 145 - DNA, 52 bp, synthetic construct
  source 1..52: mol_type = other DNA
gtggaggaatagagtaagtacaaaatcctccgttgcagtagttctccagctg

SEQ ID NO: 146 - DNA, 52 bp, synthetic construct
  source 1..52: mol_type = other DNA
tacttactctattcctccacagtgctatggatccagacattggggatcacat

SEQ ID NO: 147 - DNA, 41 bp, synthetic construct
  source 1..41: mol_type = other DNA
tcaccactacctccagaaccgtcctctgcctcacgtctggt

SEQ ID NO: 148 - DNA, 40 bp, synthetic construct
  source 1..40: mol_type = other DNA
acggttctggaggtagtggtgagaatctgtacttccaagg

SEQ ID NO: 149 - DNA, 49 bp, synthetic construct
  source 1..49: mol_type = other DNA
atattggaggaatagaataagtacacctgcattgttccacaatgccttg

SEQ ID NO: 150 - DNA, 50 bp, synthetic construct
  source 1..50: mol_type = other DNA
cttattctattcctccaatatgctttggatccctctaccagctggagaac

SEQ ID NO: 151 - DNA, 49 bp, synthetic construct
  source 1..49: mol_type = other DNA
actgtggaggaatagagtaagtacaaaagcattgttccacaatgccttg

SEQ ID NO: 152 - DNA, 50 bp, synthetic construct
  source 1..50: mol_type = other DNA
cttactctattcctccacagtgctatggatccctctaccagctggagaac

SEQ ID NO: 153 - DNA, 78 bp, synthetic construct
  source 1..78: mol_type = other DNA
ccagctggagaactactgcggatccagacattggggatcacatcatcaccatcatcatcactagtgtgtatagtcaat

SEQ ID NO: 154 - DNA, 25 bp, synthetic construct
  source 1..25: mol_type = other DNA
gcggataacaatttgatgtgctagc

SEQ ID NO: 155 - DNA, 52 bp, synthetic construct
  source 1..52: mol_type = other DNA
taccttcttcgattttcatatgcctaccttcgatcactttgtgattcatggt

SEQ ID NO: 156 - DNA, 59 bp, synthetic construct
  source 1..59: mol_type = other DNA
cggtaccctcgagggatccgagaatctgtacttccaaggaggcgagagcctgttcaagg

SEQ ID NO: 157 - DNA, 69 bp, synthetic construct
  source 1..69: mol_type = other DNA
cctatctagactgcaggtcgacctaatgatgatgatgatgatgacctccattcatcagctgagtggcct

SEQ ID NO: 158 - DNA, 708 bp, synthetic construct
  source 1..708: mol_type = other DNA
ggcgagagcctgttcaagggccctcgtgactacaaccctatcagtagcacaatttgtcacctgaccaacgagagtgatggccacacaacaagcctgtacggcatcgggttcggcccctttatcatcaccaacaagcacctgttccgtcgcaataatggcactctgctggtgcagagcctgcacggggtgttcaaagtgaagaacacaaccactctgcagcagcacctgatcgatgggcgcgatatgatcatcattcgtatgcccaaggacttccccccttttcctcagaaactgaagttccgtgagccccagcgcgaggagcgcatctgtctggtgaccacaaactttcagactaagagcatgtccagcatggtgagcgatactagctgtaccttcccatcatctgacggcatcttctggaagcactggattcagactaaggacggccagtgtggcagcccactggtgagcacacgtgacggcttcatcgtggggattcacagcgcctccaactttacaaacaccaataactatttcacctcagtgccaaagaactttatggagctgctgaccaaccaggaggcccagcagtgggtgagcgggtggcgcctgaacgccgattccgtgctgtggggcgggcacaaggtgtttatggtgaagccagaggagcctttccagcctgtgaaggaggccactcagctgatgaat

SEQ ID NO: 159 - DNA, 18 bp, synthetic construct
  source 1..18: mol_type = other DNA
gcggtaggcgtgtacggt

SEQ ID NO: 160 - DNA, 19 bp, synthetic construct
  source 1..19: mol_type = other DNA
ccggacacgctgaacttgt

SEQ ID NO: 161 - DNA, 40 bp, synthetic construct
  source 1..40: mol_type = other DNA
caccactacctccagaaccgggaattggaatgttggcctc

SEQ ID NO: 162 - DNA, 40 bp, synthetic construct
  source 1..40: mol_type = other DNA
cggttctggaggtagtggtggagatttccacatggaagag

SEQ ID NO: 163 - DNA, 59 bp, synthetic construct
  source 1..59: mol_type = other DNA
cggtaccctcgagggatccgagaatctgtacttccaaggatccaatgaatgtcttggta

SEQ ID NO: 164 - DNA, 69 bp, synthetic construct
  source 1..69: mol_type = other DNA
cctatctagactgcaggtcgacctaatgatgatgatgatgatgacctccttcaacaactgcctcggctt

SEQ ID NO: 165 - protein, 120 aa, synthetic construct
ENLYFQGRCTYSIPPICFGGFVNQHLCGSHLVEALYLVCGERGFFYTPKTRREAEDLQVGQVELGGGPGAGSLQPLALGENLYFQGIVEQCCTSICSLYQLENYCNGSRHWGSHHHHHHH

SEQ ID NO: 166 - protein, 119 aa, synthetic construct
ENLYFQFCTYSIPPQCYGGFVNQHLCGSHLVEALYLVCGERGFFYTPKTRREAEDLQVGQVELGGGPGAGSLQPLALGENLYFQGIVEQCCTSICSLYQLENYCNGSRHWGSHHHHHHH

SEQ ID NO: 167 - protein, 120 aa, synthetic construct
ENLYFQGRCTYSIPPICFGGFVNQHLCGSHLVEALYLVCGERGFFYTDKTRREAEDLQVGQVELGGGPGAGSLQPLALGENLYFQGIVEQCCTSICSLYQLENYCNGSRHWGSHHHHHHH

SEQ ID NO: 168 - protein, 119 aa, synthetic construct
ENLYFQFCTYSIPPQCYGGFVNQHLCGSHLVEALYLVCGERGFFYTDKTRREAEDLQVGQVELGGGPGAGSLQPLALGENLYFQGIVEQCCTSICSLYQLENYCNGSRHWGSHHHHHHH

SEQ ID NO: 169 - protein, 120 aa, synthetic construct
ENLYFQGRCTYSIPPICFGGFVNQHLCGSHLVEALYLVCGERGFFYTKPTRREAEDLQVGQVELGGGPGAGSLQPLALGENLYFQGIVEQCCTSICSLYQLENYCNGSRHWGSHHHHHHH

SEQ ID NO: 170 - protein, 119 aa, synthetic construct
ENLYFQFCTYSIPPQCYGGFVNQHLCGSHLVEALYLVCGERGFFYTKPTRREAEDLQVGQVELGGGPGAGSLQPLALGENLYFQGIVEQCCTSICSLYQLENYCNGSRHWGSHHHHHHH

SEQ ID NO: 171 - protein, 119 aa, synthetic construct
ENLYFQGRCTYSIPPICFGGFVNQHLCGSHLVEALYLVCGERGFFYTKPTRREAEDLQVGQVELGGGPGAGSLQPLALGENLYFQGIVEQCCTSICSLYQLENYCGSRHWGSHHHHHHH

SEQ ID NO: 172 - protein, 118 aa, synthetic construct
ENLYFQFCTYSIPPQCYGGFVNQHLCGSHLVEALYLVCGERGFFYTKPTRREAEDLQVGQVELGGGPGAGSLQPLALGENLYFQGIVEQCCTSICSLYQLENYCGSRHWGSHHHHHHH

SEQ ID NO: 173 - protein, 120 aa, synthetic construct
ENLYFQGRCTYSIPPICFGGFVNQHLCGSHLVEALYLVCGEEGFFYTKPTRREAEDLQVGQVELGGGPGAGSLQPLALGENLYFQGIVEQCCTSICSLYQLENYCNGSRHWGSHHHHHHH

SEQ ID NO: 174 - protein, 119 aa, synthetic construct
ENLYFQFCTYSIPPQCYGGFVNQHLCGSHLVEALYLVCGEEGFFYTKPTRREAEDLQVGQVELGGGPGAGSLQPLALGENLYFQGIVEQCCTSICSLYQLENYCNGSRHWGSHHHHHHH

SEQ ID NO: 175 - DNA, 364 bp, synthetic construct
  source 1..364: mol_type = other DNA
gagaatctgtacttccaaggtaggtgtacttattctattcctccaatatgctttggaggttttgtgaaccaacacctgtgcggctcacacctggtggaagctctgtacctggtgtgcggagaacgtggattcttctacacaccaaagaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 176 - DNA, 361 bp, synthetic construct
  source 1..361: mol_type = other DNA
gagaatctgtacttccaattttgtacttactctattcctccacagtgctatggaggttttgtgaaccaacacctgtgcggctcacacctggtggaagctctgtacctggtgtgcggagaacgtggattcttctacacaccaaagaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 177 - DNA, 364 bp, synthetic construct
  source 1..364: mol_type = other DNA
gagaatctgtacttccaaggtaggtgtacttattctattcctccaatatgctttggaggttttgtgaaccaacacctgtgcggctcacacctggtggaagctctgtacctggtgtgcggagaacgtggattcttctacacagataagaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 178 - DNA, 361 bp, synthetic construct
  source 1..361: mol_type = other DNA
gagaatctgtacttccaattttgtacttactctattcctccacagtgctatggaggttttgtgaaccaacacctgtgcggctcacacctggtggaagctctgtacctggtgtgcggagaacgtggattcttctacacagataagaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 179 - DNA, 364 bp, synthetic construct
  source 1..364: mol_type = other DNA
gagaatctgtacttccaaggtaggtgtacttattctattcctccaatatgctttggaggttttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 180 - DNA, 361 bp, synthetic construct
  source 1..361: mol_type = other DNA
gagaatctgtacttccaattttgtacttactctattcctccacagtgctatggaggttttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 181 - DNA, 361 bp, synthetic construct
  source 1..361: mol_type = other DNA
gagaatctgtacttccaaggtaggtgtacttattctattcctccaatatgctttggaggttttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcggatccagacattggggatcacatcatcaccatcatcatcactagt

SEQ ID NO: 182 - DNA, 358 bp, synthetic construct
  source 1..358: mol_type = other DNA
gagaatctgtacttccaattttgtacttactctattcctccacagtgctatggaggttttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcggatccagacattggggatcacatcatcaccatcatcatcactagt

SEQ ID NO: 183 - DNA, 364 bp, synthetic construct
  source 1..364: mol_type = other DNA
gagaatctgtacttccaaggtaggtgtacttattctattcctccaatatgctttggaggttttgtgaaccaacacctgtgcggctcacacctggtggaagctctgtacctggtgtgcggagaagagggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 184 - DNA, 361 bp, synthetic construct
  source 1..361: mol_type = other DNA
gagaatctgtacttccaattttgtacttactctattcctccacagtgctatggaggttttgtgaaccaacacctgtgcggctcacacctggtggaagctctgtacctggtgtgcggagaagagggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 185 - protein, 109 aa, synthetic construct
ENLYFQFVNQHLCGSHLVEALYLVCGERGFFYTPKTGRCTYSIPPICFGSRHWGGGPGAGSLQPLALGENLYFQGIVEQCCTSICSLYQLENYCNGSRHWGSHHHHHHH

SEQ ID NO: 186 - protein, 109 aa, synthetic construct
ENLYFQFVNQHLCGSHLVEALYLVCGERGFFYTPKTGFCTYSIPPQCYGSRHWGGGPGAGSLQPLALGENLYFQGIVEQCCTSICSLYQLENYCNGSRHWGSHHHHHHH

SEQ ID NO: 187 - protein, 109 aa, synthetic construct
ENLYFQFVNQHLCGSHLVEALYLVCGERGFFYTDKTGRCTYSIPPICFGSRHWGGGPGAGSLQPLALGENLYFQGIVEQCCTSICSLYQLENYCNGSRHWGSHHHHHHH

SEQ ID NO: 188 - protein, 109 aa, synthetic construct
ENLYFQFVNQHLCGSHLVEALYLVCGERGFFYTDKTGFCTYSIPPQCYGSRHWGGGPGAGSLQPLALGENLYFQGIVEQCCTSICSLYQLENYCNGSRHWGSHHHHHHH

SEQ ID NO: 189 - protein, 109 aa, synthetic construct
ENLYFQFVNQHLCGSHLVEALYLVCGERGFFYTKPTGRCTYSIPPICFGSRHWGGGPGAGSLQPLALGENLYFQGIVEQCCTSICSLYQLENYCNGSRHWGSHHHHHHH

SEQ ID NO: 190 - protein, 109 aa, synthetic construct
ENLYFQFVNQHLCGSHLVEALYLVCGERGFFYTKPTGFCTYSIPPQCYGSRHWGGGPGAGSLQPLALGENLYFQGIVEQCCTSICSLYQLENYCNGSRHWGSHHHHHHH

SEQ ID NO: 191 - protein, 109 aa, synthetic construct
ENLYFQFVNQHLCGSHLVEALYLVCGEEGFFYTKPTGRCTYSIPPICFGSRHWGGGPGAGSLQPLALGENLYFQGIVEQCCTSICSLYQLENYCNGSRHWGSHHHHHHH

SEQ ID NO: 192 - protein, 109 aa, synthetic construct
ENLYFQFVNQHLCGSHLVEALYLVCGEEGFFYTKPTGFCTYSIPPQCYGSRHWGGGPGAGSLQPLALGENLYFQGIVEQCCTSICSLYQLENYCNGSRHWGSHHHHHHH

SEQ ID NO: 193 - protein, 101 aa, synthetic construct
ENLYFQFVNQHLCGSHLVEALYLVCGERGFFYTKPTGRCTYSIPPICFGSRHWGGSGGSGENLYFQGIVEQCCTSICSLYQLENYCNGSRHWGSHHHHHHH

SEQ ID NO: 194 - protein, 101 aa, synthetic construct
ENLYFQFVNQHLCGSHLVEALYLVCGERGFFYTKPTGFCTYSIPPQCYGSRHWGGSGGSGENLYFQGIVEQCCTSICSLYQLENYCNGSRHWGSHHHHHHH

SEQ ID NO: 195 - protein, 108 aa, synthetic construct
ENLYFQFVNQHLCGSHLVEALYLVCGERGFFYTKPTGRCTYSIPPICFGSRHWGGGPGAGSLQPLALGENLYFQGIVEQCCTSICSLYQLENYCGSRHWGSHHHHHHH

SEQ ID NO: 196 - protein, 108 aa, synthetic construct
ENLYFQFVNQHLCGSHLVEALYLVCGERGFFYTKPTGFCTYSIPPQCYGSRHWGGGPGAGSLQPLALGENLYFQGIVEQCCTSICSLYQLENYCGSRHWGSHHHHHHH

SEQ ID NO: 197 - DNA, 331 bp, synthetic construct
  source 1..331: mol_type = other DNA
gagaatctgtacttccaatttgtgaaccaacacctgtgcggctcacacctggtggaagctctgtacctggtgtgcggagaacgtggattcttctacacaccaaagaccggtaggtgtacttattctattcctccaatatgctttggatccagacattggggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 198 - DNA, 331 bp, synthetic construct
  source 1..331: mol_type = other DNA
gagaatctgtacttccaatttgtgaaccaacacctgtgcggctcacacctggtggaagctctgtacctggtgtgcggagaacgtggattcttctacacaccaaagaccggattttgtacttactctattcctccacagtgctatggatccagacattggggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 199 - DNA, 331 bp, synthetic construct
  source 1..331: mol_type = other DNA
gagaatctgtacttccaatttgtgaaccaacacctgtgcggctcacacctggtggaagctctgtacctggtgtgcggagaacgtggattcttctacacagataagaccggtaggtgtacttattctattcctccaatatgctttggatccagacattggggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 200 - DNA, 331 bp, synthetic construct
  source 1..331: mol_type = other DNA
gagaatctgtacttccaatttgtgaaccaacacctgtgcggctcacacctggtggaagctctgtacctggtgtgcggagaacgtggattcttctacacagataagaccggattttgtacttactctattcctccacagtgctatggatccagacattggggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 201 - DNA, 331 bp, synthetic construct
  source 1..331: mol_type = other DNA
gagaatctgtacttccaatttgtgaaccaacacctgtgcggctcacacctggtggaagctctgtacctggtgtgcggagaacgtggattcttctacacaaagccaaccggtaggtgtacttattctattcctccaatatgctttggatccagacattggggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 202 - DNA, 331 bp, synthetic construct
  source 1..331: mol_type = other DNA
gagaatctgtacttccaatttgtgaaccaacacctgtgcggctcacacctggtggaagctctgtacctggtgtgcggagaacgtggattcttctacacaaagccaaccggattttgtacttactctattcctccacagtgctatggatccagacattggggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 203 - DNA, 331 bp, synthetic construct
  source 1..331: mol_type = other DNA
gagaatctgtacttccaatttgtgaaccaacacctgtgcggctcacacctggtggaagctctgtacctggtgtgcggagaagagggattcttctacacaaagccaaccggtaggtgtacttattctattcctccaatatgctttggatccagacattggggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 204 - DNA, 331 bp, synthetic construct
  source 1..331: mol_type = other DNA
gagaatctgtacttccaatttgtgaaccaacacctgtgcggctcacacctggtggaagctctgtacctggtgtgcggagaagagggattcttctacacaaagccaaccggattttgtacttactctattcctccacagtgctatggatccagacattggggaggtggtcctggtgcaggcagcctgcagccattggcactgggggagaatctgtacttccaaggcattgtggaacaatgctgtaccagcatctgctccctctaccagctggagaactactgcaacggatccagacattggggatcacatcatcaccatcatcatcactagt

SEQ ID NO: 205 - DNA, 307 bp, synthetic construct
  source 1..307: mol_type = other DNA
gagaatctgtacttccaatttgtgaaccaacacctgtgcggctcacacctggtggaagctctgtacctggtgtgcggagaacgtggattcttctacacaaagccaaccggtaggtgtacttattctattcctccaatatgctttggatccagacattggggaggttctggaggtagtggtgagaatctgtacttccaaggcattgtggaacaatgctgtaccagcatctgctccctctaccagctggagaactactgcaacggatccagacattggggatcacatcatcaccatcatcatcactagt

SEQ ID NO: 206 - DNA, 307 bp, synthetic construct
  source 1..307: mol_type = other DNA
gagaatctgtacttccaatttgtgaaccaacacctgtgcggctcacacctggtggaagctctgtacctggtgtgcggagaacgtggattcttctacacaaagccaaccggattttgtacttactctattcctccacagtgctatggatccagacattggggaggttctggaggtagtggtgagaatctgtacttccaaggcattgtggaacaatgctgtaccagcatctgctccctctaccagctggagaactactgcaacggatccagacattggggatcacatcatcaccatcatcatcactagt

SEQ ID NO: 207 - DNA, 328 bp, synthetic construct
  source 1..328: mol_type = other DNA
gagaatctgtacttccaatttgtgaaccaacacctgtgcggctcacacctggtggaagctctgtacctggtgtgcggagaacgtggattcttctacacaaagccaaccggtaggtgtacttattctattcctccaatatgctttggatccagacattggggaggtggtcctggtgcaggcagcctgcagccattggcactgggggagaatctgtacttccaaggcattgtggaacaatgctgtaccagcatctgctccctctaccagctggagaactactgcggatccagacattggggatcacatcatcaccatcatcatcactagt

SEQ ID NO: 208 - DNA, 328 bp, synthetic construct
  source 1..328: mol_type = other DNA
gagaatctgtacttccaatttgtgaaccaacacctgtgcggctcacacctggtggaagctctgtacctggtgtgcggagaacgtggattcttctacacaaagccaaccggattttgtacttactctattcctccacagtgctatggatccagacattggggaggtggtcctggtgcaggcagcctgcagccattggcactgggggagaatctgtacttccaaggcattgtggaacaatgctgtaccagcatctgctccctctaccagctggagaactactgcggatccagacattggggatcacatcatcaccatcatcatcactagt

SEQ ID NO: 209 - protein, 119 aa, synthetic construct
ENLYFQFVNQHLCGSHLVEALYLVCGERGFFYTPKTRREAEDLQVGQVELGGGPGAGSLQPLALGENLYFQGRCTYSIPPICFGGIVEQCCTSICSLYQLENYCNGSRHWGSHHHHHHH

SEQ ID NO: 210 - protein, 119 aa, synthetic construct
ENLYFQFVNQHLCGSHLVEALYLVCGERGFFYTPKTRREAEDLQVGQVELGGGPGAGSLQPLALGENLYFQGFCTYSIPPQCYGGIVEQCCTSICSLYQLENYCNGSRHWGSHHHHHHH

SEQ ID NO: 211 - protein, 119 aa, synthetic construct
ENLYFQFVNQHLCGSHLVEALYLVCGERGFFYTDKTRREAEDLQVGQVELGGGPGAGSLQPLALGENLYFQGRCTYSIPPICFGGIVEQCCTSICSLYQLENYCNGSRHWGSHHHHHHH

SEQ ID NO: 212 - protein, 119 aa, synthetic construct
ENLYFQFVNQHLCGSHLVEALYLVCGERGFFYTDKTRREAEDLQVGQVELGGGPGAGSLQPLALGENLYFQGFCTYSIPPQCYGGIVEQCCTSICSLYQLENYCNGSRHWGSHHHHHHH

SEQ ID NO: 213 - protein, 119 aa, synthetic construct
ENLYFQFVNQHLCGSHLVEALYLVCGERGFFYTKPTRREAEDLQVGQVELGGGPGAGSLQPLALGENLYFQGRCTYSIPPICFGGIVEQCCTSICSLYQLENYCNGSRHWGSHHHHHHH

SEQ ID NO: 214 - protein, 119 aa, synthetic construct
ENLYFQFVNQHLCGSHLVEALYLVCGERGFFYTKPTRREAEDLQVGQVELGGGPGAGSLQPLALGENLYFQGFCTYSIPPQCYGGIVEQCCTSICSLYQLENYCNGSRHWGSHHHHHHH

SEQ ID NO: 215 - protein, 119 aa, synthetic construct
ENLYFQFVNQHLCGSHLVEALYLVCGEEGFFYTKPTRREAEDLQVGQVELGGGPGAGSLQPLALGENLYFQGRCTYSIPPICFGGIVEQCCTSICSLYQLENYCNGSRHWGSHHHHHHH

SEQ ID NO: 216 - protein, 119 aa, synthetic construct
ENLYFQFVNQHLCGSHLVEALYLVCGEEGFFYTKPTRREAEDLQVGQVELGGGPGAGSLQPLALGENLYFQGFCTYSIPPQCYGGIVEQCCTSICSLYQLENYCNGSRHWGSHHHHHHH

SEQ ID NO: 217 - protein, 118 aa, synthetic construct
ENLYFQFVNQHLCGSHLVEALYLVCGERGFFYTKPTRREAEDLQVGQVELGGGPGAGSLQPLALGENLYFQGRCTYSIPPICFGGIVEQCCTSICSLYQLENYCGSRHWGSHHHHHHH

SEQ ID NO: 218 - protein, 118 aa, synthetic construct
ENLYFQFVNQHLCGSHLVEALYLVCGERGFFYTKPTRREAEDLQVGQVELGGGPGAGSLQPLALGENLYFQGFCTYSIPPQCYGGIVEQCCTSICSLYQLENYCGSRHWGSHHHHHHH

SEQ ID NO: 219 - DNA, 361 bp, synthetic construct
  source 1..361: mol_type = other DNA
gagaatctgtacttccaatttgtgaaccaacacctgtgcggctcacacctggtggaagctctgtacctggtgtgcggagaacgtggattcttctacacaccaaagaccagacgtgaggcagaggacttgcaggtgggacaggtggagctgggaggtggtcctggtgcaggcagcctgcagccattggcactgggggagaatctgtacttccaaggtaggtgtacttattctattcctccaatatgctttggaggcattgtggaacaatgctgtaccagcatctgctccctctaccagctggagaactactgcaacggatccagacattggggatcacatcatcaccatcatcatcactagt

SEQ ID NO: 220 - DNA, 361 bp, synthetic construct
  source 1..361: mol_type = other DNA
gagaatctgtacttccaatttgtgaaccaacacctgtgcggctcacacctggtggaagctctgtacctggtgtgcggagaacgtggattcttctacacaccaaagaccagacgtgaggcagaggacttgcaggtgggacaggtggagctgggaggtggtcctggtgcaggcagcctgcagccattggcactgggggagaatctgtacttccaaggattttgtacttactctattcctccacagtgctatggaggcattgtggaacaatgctgtaccagcatctgctccctctaccagctggagaactactgcaacggatccagacattggggatcacatcatcaccatcatcatcactagt

SEQ ID NO: 221 - DNA, 361 bp, synthetic construct
  source 1..361: mol_type = other DNA
gagaatctgtacttccaatttgtgaaccaacacctgtgcggctcacacctggtggaagctctgtacctggtgtgcggagaacgtggattcttctacacagataagaccagacgtgaggcagaggacttgcaggtgggacaggtggagctgggaggtggtcctggtgcaggcagcctgcagccattggcactgggggagaatctgtacttccaaggtaggtgtacttattctattcctccaatatgctttggaggcattgtggaacaatgctgtaccagcatctgctccctctaccagctggagaactactgcaacggatccagacattggggatcacatcatcaccatcatcatcactagt

SEQ ID NO: 222 - DNA, 361 bp, synthetic construct
  source 1..361: mol_type = other DNA
gagaatctgtacttccaatttgtgaaccaacacctgtgcggctcacacctggtggaagctctgtacctggtgtgcggagaacgtggattcttctacacagataagaccagacgtgaggcagaggacttgcaggtgggacaggtggagctgggaggtggtcctggtgcaggcagcctgcagccattggcactgggggagaatctgtacttccaaggattttgtacttactctattcctccacagtgctatggaggcattgtggaacaatgctgtaccagcatctgctccctctaccagctggagaactactgcaacggatccagacattggggatcacatcatcaccatcatcatcactagt

SEQ ID NO: 223 - DNA, 361 bp, synthetic construct
  source 1..361: mol_type = other DNA
gagaatctgtacttccaatttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggtaggtgtacttattctattcctccaatatgctttggaggcattgtggaacaatgctgtaccagcatctgctccctctaccagctggagaactactgcaacggatccagacattggggatcacatcatcaccatcatcatcactagt

SEQ ID NO: 224 - DNA, 361 bp, synthetic construct
  source 1..361: mol_type = other DNA
gagaatctgtacttccaatttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggattttgtacttactctattcctccacagtgctatggaggcattgtggaacaatgctgtaccagcatctgctccctctaccagctggagaactactgcaacggatccagacattggggatcacatcatcaccatcatcatcactagt

SEQ ID NO: 225 - DNA, 361 bp, synthetic construct
  source 1..361: mol_type = other DNA
gagaatctgtacttccaatttgtgaaccaacacctgtgcggctcacacctggtggaagctctgtacctggtgtgcggagaagagggattcttctacacaaagccaaccagacgtgaggcagaggacttgcaggtgggacaggtggagctgggaggtggtcctggtgcaggcagcctgcagccattggcactgggggagaatctgtacttccaaggtaggtgtacttattctattcctccaatatgctttggaggcattgtggaacaatgctgtaccagcatctgctccctctaccagctggagaactactgcaacggatccagacattggggatcacatcatcaccatcatcatcactagt

SEQ ID NO: 226 - DNA, 361 bp, synthetic construct
  source 1..361: mol_type = other DNA
gagaatctgtacttccaatttgtgaaccaacacctgtgcggctcacacctggtggaagctctgtacctggtgtgcggagaagagggattcttctacacaaagccaaccagacgtgaggcagaggacttgcaggtgggacaggtggagctgggaggtggtcctggtgcaggcagcctgcagccattggcactgggggagaatctgtacttccaaggattttgtacttactctattcctccacagtgctatggaggcattgtggaacaatgctgtaccagcatctgctccctctaccagctggagaactactgcaacggatccagacattggggatcacatcatcaccatcatcatcactagt

SEQ ID NO: 227 - DNA, 361 bp, synthetic construct; identical to SEQ ID NO: 224
  source 1..361: mol_type = other DNA

SEQ ID NO: 228 - DNA, 358 bp, synthetic construct
  source 1..358: mol_type = other DNA
gagaatctgtacttccaatttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggattttgtacttactctattcctccacagtgctatggaggcattgtggaacaatgctgtaccagcatctgctccctctaccagctggagaactactgcggatccagacattggggatcacatcatcaccatcatcatcactagt

SEQ ID NO: 229 - protein, 119 aa, synthetic construct
ENLYFQFVNQHLCGSHLVEALYLVCGERGFFYTPKTRREAEDLQVGQVELGGGPGAGSLQPLALGENLYFQGIVEQCCTSICSLYQLENYCNGGRCTYSIPPICFGSRHWGSHHHHHHH

SEQ ID NO: 230 - protein, 119 aa, synthetic construct
ENLYFQFVNQHLCGSHLVEALYLVCGERGFFYTPKTRREAEDLQVGQVELGGGPGAGSLQPLALGENLYFQGIVEQCCTSICSLYQLENYCNGGFCTYSIPPQCYGSRHWGSHHHHHHH

SEQ ID NO: 231 - protein, 119 aa, synthetic construct
ENLYFQFVNQHLCGSHLVEALYLVCGERGFFYTDKTRREAEDLQVGQVELGGGPGAGSLQPLALGENLYFQGIVEQCCTSICSLYQLENYCNGGRCTYSIPPICFGSRHWGSHHHHHHH

SEQ ID NO: 232 - protein, 119 aa, synthetic construct
ENLYFQFVNQHLCGSHLVEALYLVCGERGFFYTDKTRREAEDLQVGQVELGGGPGAGSLQPLALGENLYFQGIVEQCCTSICSLYQLENYCNGGFCTYSIPPQCYGSRHWGSHHHHHHH

SEQ ID NO: 233 - protein, 119 aa, synthetic construct
ENLYFQFVNQHLCGSHLVEALYLVCGERGFFYTKPTRREAEDLQVGQVELGGGPGAGSLQPLALGENLYFQGIVEQCCTSICSLYQLENYCNGGRCTYSIPPICFGSRHWGSHHHHHHH

SEQ ID NO: 234 - protein, 119 aa, synthetic construct
ENLYFQFVNQHLCGSHLVEALYLVCGERGFFYTKPTRREAEDLQVGQVELGGGPGAGSLQPLALGENLYFQGIVEQCCTSICSLYQLENYCNGGFCTYSIPPQCYGSRHWGSHHHHHHH

SEQ ID NO: 235 - protein, 119 aa, synthetic construct
ENLYFQFVNQHLCGSHLVEALYLVCGEEGFFYTKPTRREAEDLQVGQVELGGGPGAGSLQPLALGENLYFQGIVEQCCTSICSLYQLENYCNGGRCTYSIPPICFGSRHWGSHHHHHHH

SEQ ID NO: 236 - protein, 119 aa, synthetic construct
ENLYFQFVNQHLCGSHLVEALYLVCGEEGFFYTKPTRREAEDLQVGQVELGGGPGAGSLQPLALGENLYFQGIVEQCCTSICSLYQLENYCNGGFCTYSIPPQCYGSRHWGSHHHHHHH

SEQ ID NO: 237 - protein, 102 aa, synthetic construct
ENLYFQFVNQHLCGSHLVEALYLVCGERGFFYTKPTRREAEDGSGGSGENLYFQGIVEQCCTSICSLYQLENYCNGGRCTYSIPPICFGSRHWGSHHHHHHH

SEQ ID NO: 238 - protein, 102 aa, synthetic construct
ENLYFQFVNQHLCGSHLVEALYLVCGERGFFYTKPTRREAEDGSGGSGENLYFQGIVEQCCTSICSLYQLENYCNGGFCTYSIPPQCYGSRHWGSHHHHHHH

SEQ ID NO: 239 - DNA, 361 bp, synthetic construct
  source 1..361: mol_type = other DNA
gagaatctgtacttccaatttgtgaaccaacacctgtgcggctcacacctggtggaagctctgtacctggtgtgcggagaacgtggattcttctacacaccaaagaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggaggtaggtgtacttattctattcctccaatatgctttggatccagacattggggatcacatcatcaccatcatcatcactagt

SEQ ID NO: 240 - DNA, 361 bp, synthetic construct; identical to SEQ ID NO: 239
  source 1..361: mol_type = other DNA

SEQ ID NO: 241 - DNA, 361 bp, synthetic construct
  source 1..361: mol_type = other DNA
gagaatctgtacttccaatttgtgaaccaacacctgtgcggctcacacctggtggaagctctgtacctggtgtgcggagaacgtggattcttctacacagataagaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggaggtaggtgtacttattctattcctccaatatgctttggatccagacattggggatcacatcatcaccatcatcatcactagt

SEQ ID NO: 242 - DNA, 361 bp, synthetic construct
  source 1..361: mol_type = other DNA
gagaatctgtacttccaatttgtgaaccaacacctgtgcggctcacacctggtggaagctctgtacctggtgtgcggagaacgtggattcttctacacagataagaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggaggattttgtacttactctattcctccacagtgctatggatccagacattggggatcacatcatcaccatcatcatcactagt

SEQ ID NO: 243 - DNA, 361 bp, synthetic construct
  source 1..361: mol_type = other DNA
gagaatctgtacttccaatttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggaggtaggtgtacttattctattcctccaatatgctttggatccagacattggggatcacatcatcaccatcatcatcactagt

SEQ ID NO: 244 - DNA, 361 bp, synthetic construct
  source 1..361: mol_type = other DNA
gagaatctgtacttccaatttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggaggattttgtacttactctattcctccacagtgctatggatccagacattggggatcacatcatcaccatcatcatcactagt

SEQ ID NO: 245 - DNA, 361 bp, synthetic construct
  source 1..361: mol_type = other DNA
gagaatctgtacttccaatttgtgaaccaacacctgtgcggctcacacctggtggaagctctgtacctggtgtgcggagaagagggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggaggtaggtgtacttattctattcctccaatatgctttggatccagacattggggatcacatcatcaccatcatcatcactagt

SEQ ID NO: 246 - DNA, 361 bp, synthetic construct
  source 1..361: mol_type = other DNA
gagaatctgtacttccaatttgtgaaccaacacctgtgcggctcacacctggtggaagctctgtacctggtgtgcggagaagagggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgctgtaccagcatctgctccctctaccagctggagaactactgcaacggaggattttgtacttactctattcctccacagtgctatggatccagacattggggatcacatcatcaccatcatcatcactagt

SEQ ID NO: 247 - DNA, 310 bp, synthetic construct
  source 1..310: mol_type = other DNA
gagaatctgtacttccaatttgtgaaccaacacctgtgcggctcacacctggtggaagctctgtacctggtgtgcggagaacgtggattcttctacacaaagccaaccagacgtgaggcagaggacggttctggaggtagtggtgagaatctgtacttccaaggcattgtggaacaatgctgtaccagcatctgctccctctaccagctggagaactactgcaacggaggtaggtgtacttattctattcctccaatatgctttggatccagacattggggatcacatcatcaccatcatcatcactagt

SEQ ID NO: 248 - DNA, 310 bp, synthetic construct
  source 1..310: mol_type = other DNA
gagaatctgtacttccaatttgtgaaccaacacctgtgcggctcacacctggtggaagctctgtacctggtgtgcggagaacgtggattcttctacacaaagccaaccagacgtgaggcagaggacggttctggaggtagtggtgagaatctgtacttccaaggcattgtggaacaatgctgtaccagcatctgctccctctaccagctggagaactactgcaacggaggattttgtacttactctattcctccacagtgctatggatccagacattggggatcacatcatcaccatcatcatcactagt

SEQ ID NO: 249 - protein, 113 aa, synthetic construct
ENLYFQFVNQHLCGSHLVEALYLVCGERGFFYTPKTRREAEDLQVGQVELGGGPGAGSLQPLALGENLYFQGIVEQCRCTYSIPPICFGSLYQLENYCNGSRHWGSHHHHHHH

SEQ ID NO: 250 - protein, 113 aa, synthetic construct
ENLYFQFVNQHLCGSHLVEALYLVCGERGFFYTPKTRREAEDLQVGQVELGGGPGAGSLQPLALGENLYFQGIVEQCFCTYSIPPQCYGSLYQLENYCNGSRHWGSHHHHHHH

SEQ ID NO: 251 - protein, 113 aa, synthetic construct
ENLYFQFVNQHLCGSHLVEALYLVCGERGFFYTDKTRREAEDLQVGQVELGGGPGAGSLQPLALGENLYFQGIVEQCRCTYSIPPICFGSLYQLENYCNGSRHWGSHHHHHHH

SEQ ID NO: 252 - protein, 113 aa, synthetic construct
ENLYFQFVNQHLCGSHLVEALYLVCGERGFFYTDKTRREAEDLQVGQVELGGGPGAGSLQPLALGENLYFQGIVEQCFCTYSIPPQCYGSLYQLENYCNGSRHWGSHHHHHHH

SEQ ID NO: 253 - protein, 113 aa, synthetic construct
ENLYFQFVNQHLCGSHLVEALYLVCGERGFFYTKPTRREAEDLQVGQVELGGGPGAGSLQPLALGENLYFQGIVEQCRCTYSIPPICFGSLYQLENYCNGSRHWGSHHHHHHH

SEQ ID NO: 254 - protein, 113 aa, synthetic construct
ENLYFQFVNQHLCGSHLVEALYLVCGERGFFYTKPTRREAEDLQVGQVELGGGPGAGSLQPLALGENLYFQGIVEQCFCTYSIPPQCYGSLYQLENYCNGSRHWGSHHHHHHH

SEQ ID NO: 255 - protein, 113 aa, synthetic construct
ENLYFQFVNQHLCGSHLVEALYLVCGEEGFFYTKPTRREAEDLQVGQVELGGGPGAGSLQPLALGENLYFQGIVEQCRCTYSIPPICFGSLYQLENYCNGSRHWGSHHHHHHH

SEQ ID NO: 256 - protein, 113 aa, synthetic construct
ENLYFQFVNQHLCGSHLVEALYLVCGEEGFFYTKPTRREAEDLQVGQVELGGGPGAGSLQPLALGENLYFQGIVEQCFCTYSIPPQCYGSLYQLENYCNGSRHWGSHHHHHHH

SEQ ID NO: 257 - protein, 112 aa, synthetic construct
ENLYFQFVNQHLCGSHLVEALYLVCGERGFFYTKPTRREAEDLQVGQVELGGGPGAGSLQPLALGENLYFQGIVEQCRCTYSIPPICFGSLYQLENYCGSRHWGSHHHHHHH

SEQ ID NO: 258 - protein, 112 aa, synthetic construct
ENLYFQFVNQHLCGSHLVEALYLVCGERGFFYTKPTRREAEDLQVGQVELGGGPGAGSLQPLALGENLYFQGIVEQCFCTYSIPPQCYGSLYQLENYCGSRHWGSHHHHHHH

SEQ ID NO: 259 - DNA, 343 bp, synthetic construct
  source 1..343: mol_type = other DNA
gagaatctgtacttccaatttgtgaaccaacacctgtgcggctcacacctggtggaagctctgtacctggtgtgcggagaacgtggattcttctacacaccaaagaccagacgtgaggcagaggacttgcaggtgggacaggtggagctgggaggtggtcctggtgcaggcagcctgcagccattggcactgggggagaatctgtacttccaaggcattgtggaacaatgcaggtgtacttattctattcctccaatatgctttggatccctctaccagctggagaactactgcaacggatccagacattggggatcacatcatcaccatcatcatcactagt

SEQ ID NO: 260 - DNA, 343 bp, synthetic construct
  source 1..343: mol_type = other DNA
gagaatctgtacttccaatttgtgaaccaacacctgtgcggctcacacctggtggaagctctgtacctggtgtgcggagaacgtggattcttctacacaccaaagaccagacgtgaggcagaggacttgcaggtgggacaggtggagctgggaggtggtcctggtgcaggcagcctgcagccattggcactgggggagaatctgtacttccaaggcattgtggaacaatgcttttgtacttactctattcctccacagtgctatggatccctctaccagctggagaactactgcaacggatccagacattggggatcacatcatcaccatcatcatcactagt

SEQ ID NO: 261 - DNA, 343 bp, synthetic construct
  source 1..343: mol_type = other DNA
gagaatctgtacttccaatttgtgaaccaacacctgtgcggctcacacctggtggaagctctgtacctggtgtgcggagaacgtggattcttctacacagataagaccagacgtgaggcagaggacttgcaggtgggacaggtggagctgggaggtggtcctggtgcaggcagcctgcagccattggcactgggggagaatctgtacttccaaggcattgtggaacaatgcaggtgtacttattctattcctccaatatgctttggatccctctaccagctggagaactactgcaacggatccagacattggggatcacatcatcaccatcatcatcactagt

SEQ ID NO: 262 - DNA, 343 bp, synthetic construct
  source 1..343: mol_type = other DNA
gagaatctgtacttccaatttgtgaaccaacacctgtgcggctcacacctggtggaagctctgtacctggtgtgcggagaacgtggattcttctacacagataagaccagacgtgaggcagaggacttgcaggtgggacaggtggagctgggaggtggtcctggtgcaggcagcctgcagccattggcactgggggagaatctgtacttccaaggcattgtggaacaatgcttttgtacttactctattcctccacagtgctatggatccctctaccagctggagaactactgcaacggatccagacattggggatcacatcatcaccatcatcatcactagt

SEQ ID NO: 263 - DNA, 343 bp, synthetic construct
  source 1..343: mol_type = other DNA
gagaatctgtacttccaatttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgcaggtgtacttattctattcctccaatatgctttggatccctctaccagctggagaactactgcaacggatccagacattggggatcacatcatcaccatcatcatcactagt

SEQ ID NO: 264 - DNA, 343 bp, synthetic construct
  source 1..343: mol_type = other DNA
gagaatctgtacttccaatttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgcttttgtacttactctattcctccacagtgctatggatccctctaccagctggagaactactgcaacggatccagacattggggatcacatcatcaccatcatcatcactagt

SEQ ID NO: 265 - DNA, 343 bp, synthetic construct
  source 1..343: mol_type = other DNA
gagaatctgtacttccaatttgtgaaccaacacctgtgcggctcacacctggtggaagctctgtacctggtgtgcggagaagagggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgcaggtgtacttattctattcctccaatatgctttggatccctctaccagctggagaactactgcaacggatccagacattggggatcacatcatcaccatcatcatcactagt

SEQ ID NO: 266 - DNA, 343 bp, synthetic construct
  source 1..343: mol_type = other DNA
gagaatctgtacttccaatttgtgaaccaacacctgtgcggctcacacctggtggaagctctgtacctggtgtgcggagaagagggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgcttttgtacttactctattcctccacagtgctatggatccctctaccagctggagaactactgcaacggatccagacattggggatcacatcatcaccatcatcatcactagt

SEQ ID NO: 267 - DNA, 340 bp, synthetic construct
  source 1..340: mol_type = other DNA
gagaatctgtacttccaatttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgcaggtgtacttattctattcctccaatatgctttggatccctctaccagctggagaactactgcggatccagacattggggatcacatcatcaccatcatcatcactagt

SEQ ID NO: 268 - DNA, 340 bp, synthetic construct
  source 1..340: mol_type = other DNA
gagaatctgtacttccaatttgtgaaccaacacctgtgcggctcacacctggtggaagctctgtacctggtgtgcggagaacgtggattcttctacacaaagccaaccagacgtgaggcagaggacttgcaggtgggacaggtggagctgggaggtggtcctggtgcaggcagcctgcagccattggcactgggggagaatctgtacttccaaggcattgtggaacaatgcttttgtacttactctattcctccacagtgctatggatccctctaccagctggagaactactgcggatccagacattggggatcacatcatcaccatcatcatcactagt

(19) United States
(12) Patent Application Publication
(10) Pub. No.: US 2026/0242438 A1
(43) Pub. Date: Aug. 20, 2026

(54) INSULIN HYBRID PEPTIDE COMPRISING CHYMOTRYPSIN INHIBITORY PEPTIDE, AND USE THEREOF

(71) Applicant: INSTITUTE OF MATERIA MEDICA, CHINESE ACADEMY OF MEDICAL SCIENCES, Beijing (CN)

(72) Inventors: Wei WANG, Beijing (CN); Zhufang SHEN, Beijing (CN); Minzhi LIU, Beijing (CN); Sihan ZHOU, Beijing (CN); Caina LI, Beijing (CN); Xin SHEN, Beijing (CN); Hui CAO, Beijing (CN); Lei LEI, Beijing (CN); Yan YANG, Beijing (CN)

(21) Appl. No.: 18/870,356

(22) PCT Filed: May 29, 2023
(86) PCT No.: PCT/CN2023/096851

§ 371 Date: Nov. 27, 2024

(30) Foreign Application Priority Data
May 29, 2022 (CN) 202210594279.5

(51) Int. Cl. C07K 14/62; A61K 38/00; A61P 3/10; C12P 21/02

(52) U.S. Cl. CPC C07K 14/62; A61P 3/10; C12P 21/02; A61K 38/00; C07K 2319/70

(57) ABSTRACT

The present invention belongs to the field of protein engineering, and relates to a hybrid peptide formed by a chymotrypsin inhibitory peptide and an insulin or an analog thereof. The hybrid insulin analogue can resist the degradation of the chymotrypsin to improve the stability, such that the curative effect of direct injection administration can be improved, and moreover, the insulin analogue can penetrate through small intestine epithelium to be directly absorbed and enter blood circulation, thereby achieving oral administration of insulin hybrid peptidedrugs. The present invention further provides a method for preparing a hybrid peptide comprising the chymotrypsin inhibiting peptide and insulin or an analog thereof.

### FIELD OF THE INVENTION

The invention belongs to the field of protein engineering, and relates to polypeptide molecules with chymotrypsin inhibition activity. The invention also relates to hybrid peptides formed by fusion of chymotrypsin inhibitory peptides and insulin molecules or analogs modified by PEGylation, phosphorylation, amidation or acylation, or pharmaceutically acceptable salts thereof. The insulin hybrid peptide still maintains the inhibitory activity against chymotrypsin, so that the stability and curative effect of in-vivo administration of the insulin hybrid peptide are improved, the degradation of small intestine metabolic enzymes can be resisted, and the insulin hybrid peptide is promoted to be directly absorbed into blood circulation through small intestine absorption, so that the oral administration of the hybrid peptide of insulin or the analog thereof is realized.

### BACKGROUND OF THE INVENTION

Insulin is a hormone secreted by the pancreas that controls blood sugar levels. It is used to transport excess glucose from the bloodstream to cells, providing an energy source and maintaining normal blood sugar levels. However, due to insulin deficiency, insulin resistance and loss of β-cell function, diabetic patients cannot maintain normal insulin function. Therefore, the diabetic patient cannot utilize glucose in blood as an energy source, but exhibits a hyperglycemia symptom with high glucose level, and then glucose is excreted in the urine, so that various complications are caused. Therefore, patients with abnormal insulin secretion (Type I diabetes) or insulin resistance (Type II diabetes) essentially require insulin therapy, and they can keep the blood glucose level normal by insulin injecting.

Due to the extremely short half-life of insulin in the body, like other proteins and peptide hormones, it cannot demonstrate sustained therapeutic effects. Therefore, there is a problem that insulin must be continuously and repeatedly administered to exert its effects. Frequent administration of insulin can cause severe pain and discomfort to patients. Therefore, it is necessary to improve the use of insulin in terms of patient compliance, safety, and convenience.

Due to the high molecular weight of insulin, it is easily hydrolyzed by gastric acid, pepsin, and gastrointestinal tract (GIT) enzymes, as well as physical barriers of the intestinal mucosal epithelium. These factors make it challenging that polypeptide drugs including insulin are difficult to orally administrate. To overcome these obstacles, many different studies have been conducted to achieve positive research results in improving insulin bioavailability [1,2,3]. These mainly include absorption enhancers such as sodium caprate and sodium N-[8-(2-hydroxybenzyl)amino] caprylate (SANC) [4], protease inhibitors (aprotinin, soy trypsin inhibitor, and leupeptin) [5], nanocarriers (such as polymers, lipids, and inorganic nanoparticles), as well as microneedles and microencapsulated protein drug delivery devices (such as RaniPill™ Capsule) [6]. These technologies have also been clinically tested, but the bioavailability of oral insulin seems to be only 1-2% [6,7]. The clinical trials of oral insulin have not entered intensive research, and the main reason may be related to cost-effectiveness.

Oral insulin administration can overcome degradation of gastric acid and pepsin by adopting an enteric-coating formulation technology, but there are still various protein metabolizing enzymes secreted by the pancreas in the small intestine, among which trypsin and chymotrypsin are most mainly included. Only by overcoming the degradation of small intestine proteases, the residence time of the insulin molecules in the small intestinal environment can be prolonged, and then the absorption of small intestine epithelium is promoted. Although the addition of protease inhibitors and absorption enhancers promotes absorption of insulin in the small intestine [8,9], it is still difficult to overcome the protease degradation of insulin after it enters the blood circulation.

The human insulin molecule consists of a 21 amino acid A-chain and a 30 amino acid B-chain, linked by two disulfide bonds. The structure of insulin is relatively stable against the degradation of trypsin [10], and the protease that degrades insulin in the small intestine microenvironment is mainly chymotrypsin. In order to improve the stability of insulin molecules to resist protease degradation, the prior art does not solve the problem of oral administration of insulin by adopting the substitution of D-type amino acid, PEG modification, fatty acylation and et al. Aiming at the defects in the prior art, the present invention isolates and identifies a series of inhibitory peptides against chymotrypsin, and then hybrid peptides are formed by fusion of insulin or analogs thereof and hymotrypsin inhibitoy pepides to solve the stability problem of insulin molecules being degraded by proteases.

### SUMMARY OF THE INVENTION

The invention provides a hybrid peptide formed by connection of an insulin or an analog thereof to a chymotrypsin inhibitory peptide via a linker peptide. The insulin or the analog thereof comprises a B-chain and an A-chain, which is connected via two pairs of intrachain disulfide bonds, the chymotrypsin inhibitory peptide is connected to the B-chain or the A-chain of insulin or the analog thereof via the linker peptide. As shown in FIG. 10, the present invention provides hybrid peptides formed by connection between five types of chymotrypsin inhibitory peptides and the B-chain or the A-chain of the insulin or the analog thereof.

In a specific embodiment of the present invention, the chymotrypsin inhibitory peptide is screened and identified by an in vitro enzymatic method, selected from the group consisting of polypeptides with the following amino acid sequences: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 11, SEQ ID NO: 12, SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28.

In one aspect of the present invention, the insulin molecules or analogs thereof including A-chain and B-chain are provided for the synthesis of hybrid peptides. The A-chain can be selected from the group consisting of peptides with the following amino acid sequences: SEQ ID NO: 29, SEQ ID NO: 30, SEQ ID NO: 31, SEQ ID NO: 32, and SEQ ID NO: 33, and the B-chain can be selected from the group consisting of polypeptides with the following amino acid sequences: SEQ ID NO: 34, SEQ ID NO: 35, SEQ ID NO: 36, SEQ ID NO: 37, SEQ ID NO: 38, SEQ ID NO: 39, and SEQ ID NO: 40.

Another aspect of the present invention provides a linker peptide linked between the chymotrypsin inhibitory peptide and the A-chain or B-chain of insulin or analogues thereof. The linker peptides contain three amino acid residues of Gly, Ser and Pro, the length of which is one or more amino acid residues, or absent.

The present invention provides the hybrid peptide of the insulin or the analog thereof comprising a N- to C-terminal domain: (N-terminus)-chymotrypsin inhibitory peptide-insulin B-chain—insulin A-chain-(C-terminus), wherein the C-terminus of the chymotrypsin inhibitory peptide is directly fused to the N-terminus of the insulin B-chain or the analog thereof via the linker peptide. The “(N-terminus)-chymotrypsin inhibitory peptide-insulin B-chain” is a polypeptide formed by fusion of the chymotrypsin inhibitory peptide and the insulin B-chain, and is selected from the goup consisting of polypeptides with the following amino acid sequences: SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 and SEQ ID NO: 50. The A-chain is selected from the goup consisting of polypeptides with the following amino acid sequences: SEQ ID NO: 29, SEQ ID NO: 31, and SEQ ID NO: 33.

In one embodiment, the hybrid peptide of the insulin or the analog thereof provided by the present invention needs to be generated through the maturation and processing of a precursor protein. The precursor protein has a N- to C-terminal domain: (N-terminus)-chymotrypsin inhibitory peptide-insulin B-chain-C-polypeptide-insulin A-chain-(C-terminus), wherein the C-terminus of the chymotrypsin inhibitory peptide is directly fused to the N-terminus of the insulin B-chain or the analog thereof via the linker peptide. The “(N-terminus)-chymotrypsin inhibitory peptide-insulin B-chain” is a polypeptide formed by fusion of the chymotrypsin inhibitory peptide and the insulin B-chain and is selected from the goup consisting of polypeptides with the following amino acid sequences: SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 and SEQ ID NO: 50. The A-chain is selected from the goup consisting of polypeptides with the following amino acid sequences: SEQ ID NO: 29, SEQ ID NO: 31, and SEQ ID NO: 33; The C-polypeptide is selected from the goup consisting of polypeptides with the following amino acid sequence: SEQ ID NO: 70 and SEQ ID NO: 71, wherein the precursor protein needs three-step treatment including protease KEX2 digestion, tobacco etch virus (TEV) protease digestion and nickel ion-induced chemical cleavage. The treatment method can be combined in a step-by-step manner. In order to obtain the hybrid peptide of the insulin or the analog thereof which does not contain two “RR” residues at the C-terminus of B-chain, a carboxypeptidase B can be further used for treatment. To obtain the hybrid peptide of the insulin or the analog thereof with the C-terminal amidation, a peptidylglycine α-amidating monooxygenase can be further used for treatment.

In one particular embodiment, a precursor protein of the hybrid peptide of the insulin or the analog thereof is selected from the goup consisting of polypeptides with the following amino acid sequences: SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173 and SEQ ID NO: 174. These precursor proteins are treated using protease KEX2 digestion, tobacco etch virus (TEV) protease digestion and nickel ion-induced chemical cleavage, respectively. In order to obtain the hybrid peptides of the insulin or its analogues without two “RR” residues at the C-terminus of the B-chain or the analogs thereof, the carboxypeptidase B can be used for further treatment. It is also possible that the peptidylglycine a-amidating monooxygenase can be continuously used for treatment to obtain hybrid peptides with C-terminal amidation modification. For example, the hybrid peptide formed by fusion of the chymotrypsin inhibitory peptide CH57 (SEQ ID NO: 4) and CH10 (SEQ ID NO: 19) to the N-terminus of the B-chain of human insulin or analog thereof is as follows:

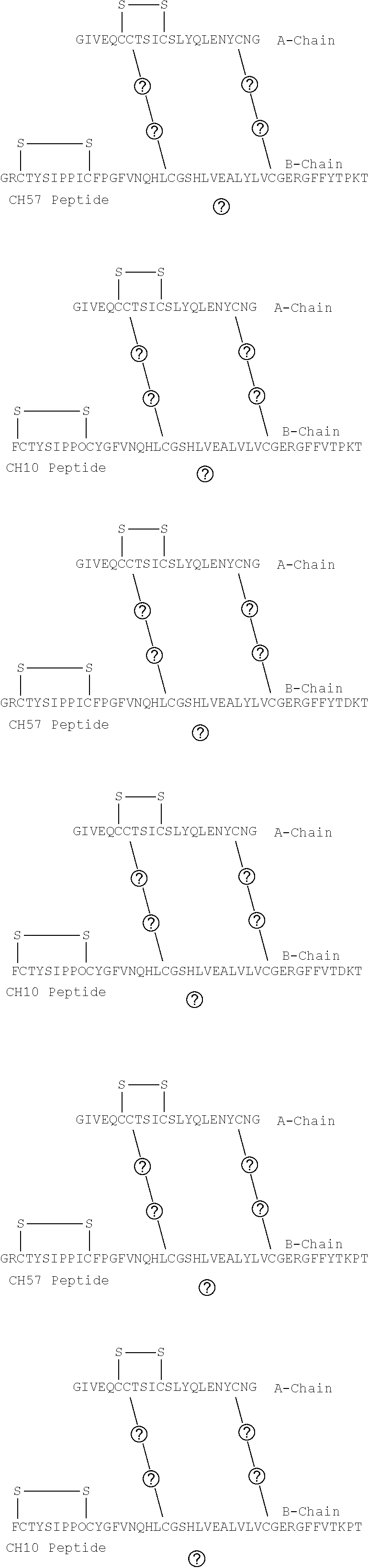

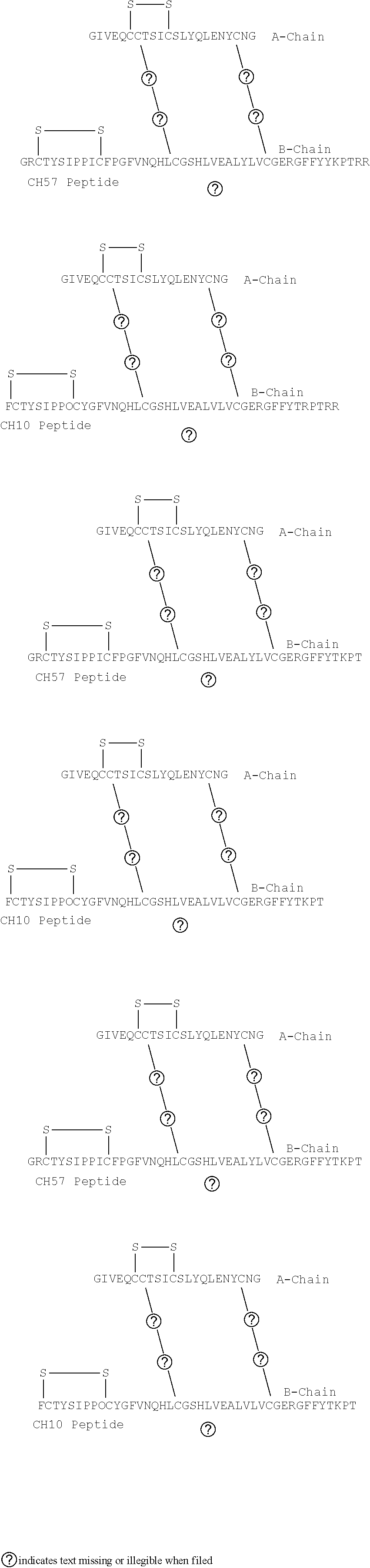

The nucleotide coding sequences of the aforementioned precursor protein of the hybrid peptides are selected from the group consisting of sequences: SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183 and SEQ ID NO: 184.

The present invention provides another hybrid peptide comprising a N- to C-terminal domain: (N-terminus)-insulin B-chain-chymotrypsin inhibitory peptide—insulin A-chain-(C-terminus), wherein the N-terminus of the chymotrypsin inhibitory peptide is directly fused to the C-terminus of the insulin B-chain or the analog thereof via the linker peptide. The “(N-terminus)-insulin B-chain-chymotrypsin inhibitory peptide” is a polypeptide formed by fusion of the insulin B-chain and the chymotrypsin inhibitory peptide and is selected from the group consisting of polypeptides with the following amino acid sequences: SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58. The A-chain is selected from the group consisting of polypeptides with the following amino acid sequences: SEQ ID NO: 29, SEQ ID NO: 31, and SEQ ID NO: 33

In one embodiment, the hybrid peptide of the insulin or the analog thereof provided by the present invention needs to be generated through the maturation and processing of a precursor protein. The precursor protein has a N- to C-terminal domain: (N-terminus)-insulin B-chain-chymotrypsin inhibitory peptide-C-polypeptide-insulin A-chain-(C-terminus), wherein the N-terminus of the chymotrypsin inhibitory peptide is fused directly to the C-terminus of the insulin B-chain or the analog thereof via the linker peptide. The “(N-terminus)-insulin B-chain-chymotrypsin inhibitory peptide” is a polypeptide formed by fusion of the insulin B-chain and the chymotrypsin inhibitory peptide, and is selected from the group consisting of polypeptides with the following amino acid sequences: SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58. The A-chain is selected from the group consisting of polypeptides with the following amino acid sequences: SEQ ID NO: 29, SEQ ID NO: 31, and SEQ ID NO: 33. The C-polypeptide is selected from the group consisting of polypeptides with the following amino acid sequences: SEQ ID NO: 72 and SEQ ID NO: 73. Wherein the precursor protein needs three-step treatment including protease KEX2 digestion, tobacco etch (TEV) protease digestion and nickel ion-induced chemical cleavage. The treatment method can be combined in a step-by-step manner. In order to obtain the hybrid peptide of the insulin or the analog thereof which does not contain two “RR” residues at the C-terminus of the B-chain, the carboxypeptidase B can be further used for treatment. To obtain the hybrid peptide of insulin or the analog thereof with the C-terminal amidation modification, the peptidylglycine a-amidating monooxygenase can be further used for treatment.

In one particular embodiment, the precursor protein of the insulin or analog hybrid peptide thereof is selected from the group consisting of polypeptides with the following amino acid sequences: SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195 [symbol] SEQ ID NO: 196. These precursor proteins are treated using protease KEX2 digestion, tobacco etch virus (TEV) protease digestion and nickel ion-induced chemical cleavage. In order to obtain the hybrid peptides of the insulin or its analogues without two “RR” residues at the C-terminus of the B-chain, the carboxypeptidase B can be used for further treatment. Additionally, the peptidylglycine a-amidating monooxygenase can be continuously used for treatment to obtain the hybrid peptides with C-terminal amidation modification. For example, the hybrid peptide formed by fusion of the chymotrypsin inhibitory peptide CH57 (SEQ ID NO: 4) and CH10 (SEQ ID NO: 19) to the C-terminus of the B-chain of human insulin or analog thereof is as follows:

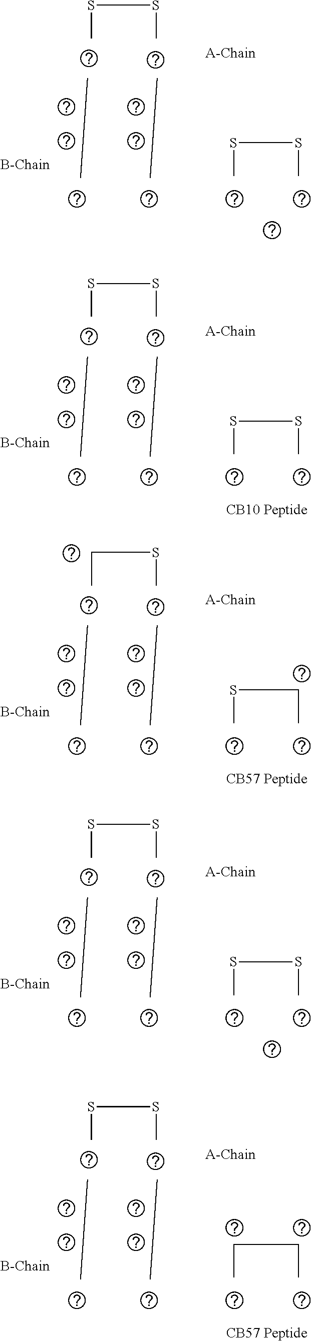

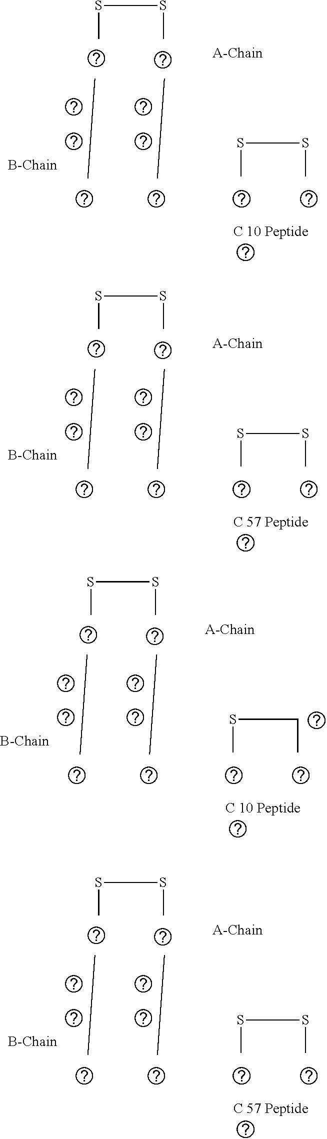

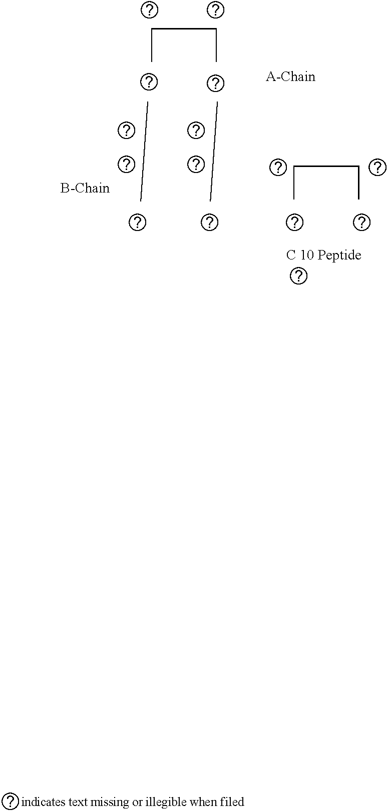

The nucleotide coding sequences of the aforementioned precursor protein of the hybrid peptide are selected from the group consisting of sequences: SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207 and SEQ ID NO: 208.

The present invention provides the hybrid peptide comprising a N- to C-terminal domain: (N-terminus)-insulin B-chain—chymotrypsin inhibitory peptide-insulin A-chain-(C-terminus), wherein the C-terminus of the chymotrypsin inhibitory peptide is directly fused to the N-terminus of the insulin A-chain or the analog thereof via the linker peptide, and the B-chain of the insulin or the analog is selected from the group consisting of polypeptides with the following amino acid sequences: SEQ ID NO: 34, SEQ ID NO: 35, SEQ ID NO: 36, SEQ ID NO: 39 and SEQ ID NO: 40; The “chymotrypsin inhibitory peptide-insulin A-chain-(C-terminus)” is a polypeptide formed by fusion of the chymotrypsin inhibitory peptide and the insulin A-chain and is selected from the group consisting of polypeptides with the following amino acid sequences: SEQ ID NO: 59, SEQ ID NO: 60, SEQ ID NO: 61 and SEQ ID NO: 62.

In one embodiment, the hybrid peptide of the insulin or the analog thereof provided by the present invention needs to be generated through the maturation and processing of a precursor protein. The precursor protein has a N- to C-terminal domain: (N-terminus)-insulin B-chain-C-polypeptide-chymotrypsin inhibitory peptide-insulin A-chain-(C-terminus), wherein the C-terminus of the chymotrypsin inhibitory peptide is directly fused to the N-terminus of the insulin A-chain or the analog thereof via the linker peptide, and the B-chain of the insulin or the analog thereof is selected from the group consisting of polypeptides with the following amino acid sequences: SEQ ID NO: 34, SEQ ID NO: 35, SEQ ID NO: 36, SEQ ID NO: 39 and SEQ ID NO: 40. The “chymotrypsin inhibitory peptide-insulin A-chain-(C-terminus)” is a polypeptide formed by fusion of the chymotrypsin inhibitory peptide and the insulin A-chain and is selected from the group consisting of polypeptides with the following amino acid sequences: SEQ ID NO: 59, SEQ ID NO: 60, SEQ ID NO: 61, and SEQ ID NO: 62. The C-polypeptide is selected from the group consisting of polypeptides with the following amino acid sequences: SEQ ID NO: 70 and SEQ ID NO: 71. Wherein the precursor protein needs three-step treatment including protease KEX2 digestion, tobacco etch virus (TEV) protease digestion and nickel ion-induced chemical cleavage. The treatment method can be combined in a step-by-step manner. In order to obtain a hybrid peptide of insulin or an analog thereof which does not contain two “RR” residues at the C-terminus of the B-chain, the carboxypeptidase B can be further used for treatment. To obtain the hybrid peptide of the insulin or the analog thereof with the C-terminal amidation modificattion, the peptidylglycine α-amidating monooxygenase can be further used for treatment.

In one particular embodiment, the precursor protein of the insulin or analog hybrid peptide thereof is selected from the group consisting of polypeptides with the following amino acid sequences: SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217 and SEQ ID NO: 218. These precursor proteins are treated using protease KEX2 digestion, tobacco etch virus (TEV) protease digestion and nickel ion-induced chemical cleavage. In order to obtain the hybrid peptides of the insulin or the analogues without two “RR” residues at the C-terminus of the B-chain, the carboxypeptidase B can be used for further treatment. Additionally, the peptidylglycine a-amidating monooxygenase can be continuously used for treatment to obtain the hybrid peptides with C-terminal amidation modification. For example, the hybrid peptide formed by fusion of the chymotrypsin inhibitory peptide CH57 (SEQ ID NO: 4) and CH10 (SEQ ID NO: 19) to the N-terminus of the A-chain of human insulin or analog thereof is as follows:

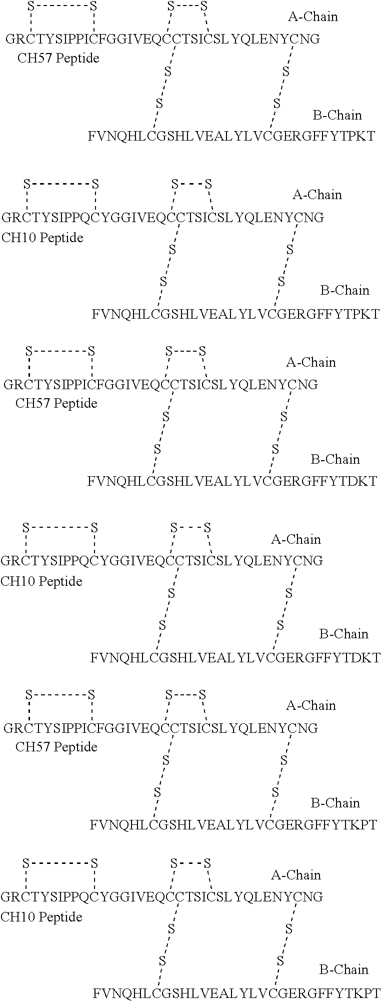

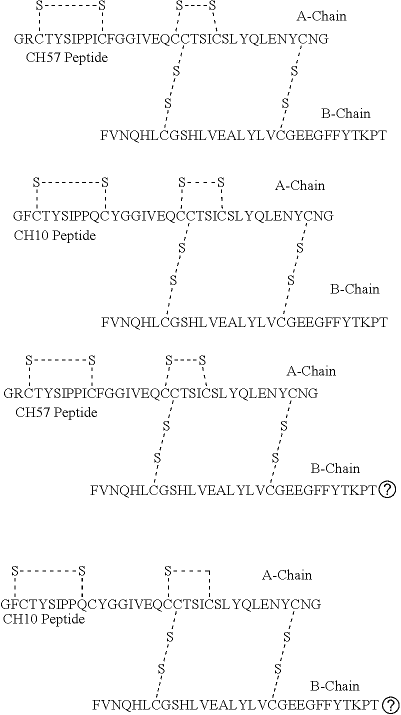

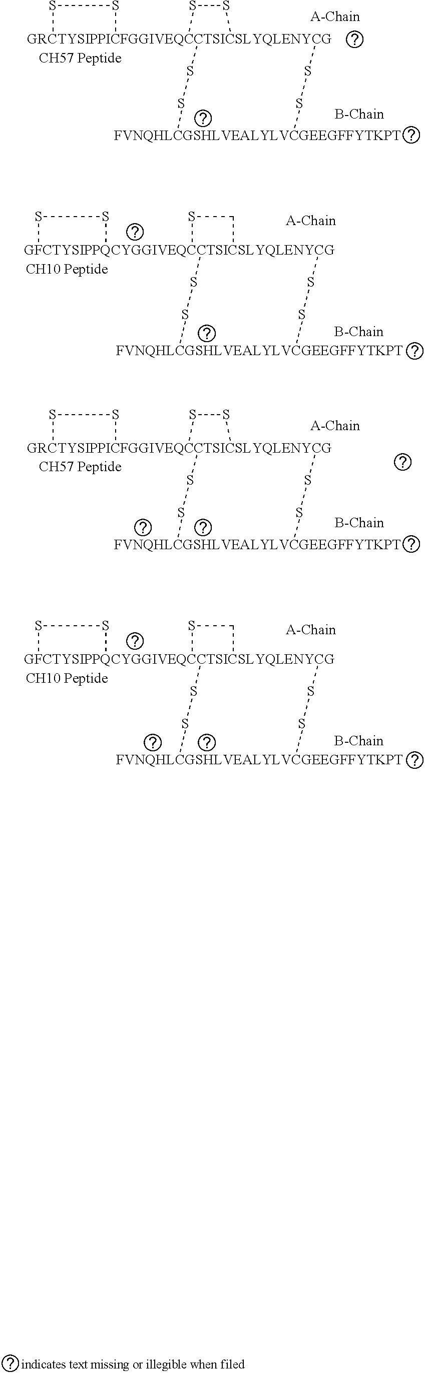

The nucleotide coding sequences of the aforementioned precursor protein of the hybrid peptide are selected from the group consisting of sequences: SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227 and SEQ ID NO: 228.

The present invention provides another hybrid peptide comprising a N- to C-terminal domain: (N-terminal)-insulin B-chain—insulin A-chain-chymotrypsin inhibitory peptide-(C-terminus), wherein the N-terminal of the chymotrypsin inhibitory peptide is fused directly to the C-terminal of the insulin A-chain or the analog thereof via the linker peptide. The B-chain of the insulin or the analog thereof is selected from the group consisting of polypeptides with the following amino acid sequence: SEQ ID NO: 34, SEQ ID NO: 35, SEQ ID NO: 36, SEQ ID NO: 39, SEQ ID NO: 40. The “insulin A-chain chymotrypsin inhibitory peptide-(C-terminus)” is a polypeptide formed by fusion of the insulin A-chain and the chymotrypsin inhibitory peptide and is selected the group consisting of from polypeptides with the following amino acid sequence: SEQ ID NO: 63 and SEQ ID NO: 64.

In another embodiment, the hybrid peptide of the insulin or the analog thereof provided by the present invention needs to be generated through the maturation and processing of a precursor protein. The precursor protein has a N- to C-terminal domain: (N-terminus)-insulin B-chain-C-polypeptide-insulin A-chain-chymotrypsin inhibitory peptide-(C-terminus), wherein the N-terminus of the chymotrypsin inhibitory peptide is directly fused to the C-terminus of the insulin A-chain or the analog thereof via the linker peptide, the B-chain of insulin or the analog thereof is selected from the group consisting of polypeptides with the following amino acid sequence: SEQ ID NO: 34, SEQ ID NO: 35, SEQ ID NO: 36, SEQ ID NO: 39, and SEQ ID NO: 40. The “insulin A-chain-chymotrypsin inhibitory peptide-(C-terminus)” is a polypeptide formed by fusion of the insulin A-chain and the chymotrypsin inhibitory peptide and is selected from the group consisting of polypeptides with the following amino acid sequence: SEQ ID NO: 63 and SEQ ID NO: 64. The C-polypeptide is selected from the group consisting of polypeptides with the following amino acid sequence: SEQ ID NO: 70 and SEQ ID NO: 71. Wherein the precursor protein needs three-step treatment including protease KEX2 digestion, tobacco etch virus (TEV) protease digestion and nickel ion-induced chemical cleavage. The treatment method can be combined in a step-by-step manner. In order to obtain the hybrid peptide of the insulin or the analog thereof which does not contain two “RR” residues at the C-terminus of the B-chain, the carboxypeptidase B can be further used for treatment. To obtain the hybrid peptide of the insulin or the analog thereof with the C-terminal amidation modification, the peptidylglycine a-amidating monooxygenase can be further used for treatment.

In one particular embodiment, the precursor protein of the insulin or analog hybrid peptide thereof is selected from the group consisting of polypeptides with the following amino acid sequences: SEQ ID NO:229, SEQ ID NO:230, SEQ ID NO: 231, SEQ ID NO: 232, SEQ ID NO: 233, SEQ ID NO: 234, SEQ ID NO: 235, SEQ ID NO: 236, SEQ ID NO: 237 and SEQ ID NO: 238. These precursor proteins are treated using protease KEX2 digestion, tobacco etch virus (TEV) protease digestion and nickel ion-induced chemical cleavage. In order to obtain the hybrid peptides of the insulin or the analogues without two “RR” residues at the C-terminus of the B-chain, the carboxypeptidase B can be used for further treatment. Additionally, the peptidylglycine a-amidating monooxygenase can be continuously used for treatment to obtain the hybrid peptides with C-terminal amidation modification. For example, the hybrid peptide formed by fusion of the chymotrypsin inhibitory peptide CH57 (SEQ ID NO: 4) and CH10 (SEQ ID NO: 19) to the C-terminus of the A-chain of human insulin or analog thereof is as follows:

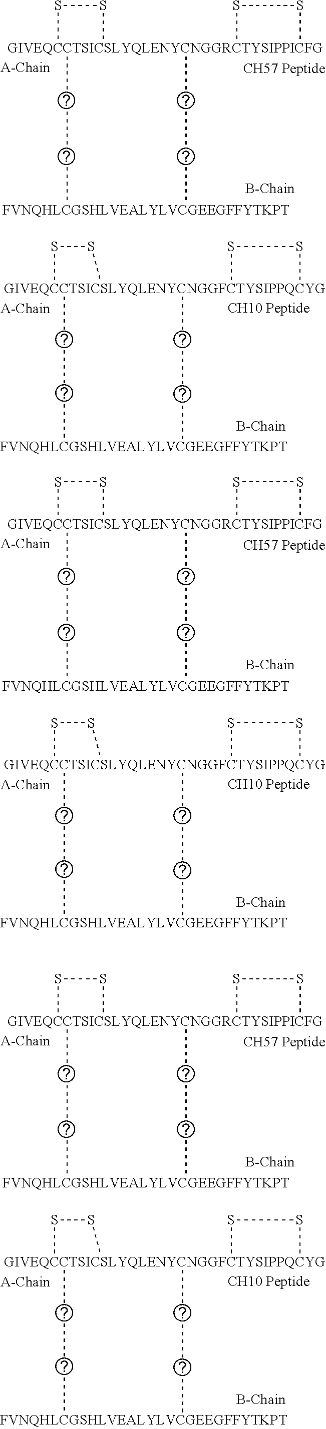

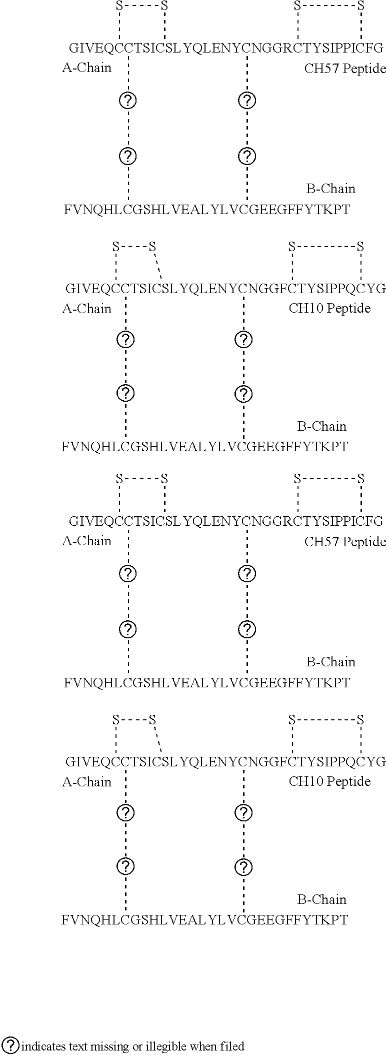

The nucleotide coding sequences of the aforementioned precursor protein of the hybrid peptide are selected from the group consisting of sequences: SEQ ID NO:239, SEQ ID NO:240, SEQ ID NO: 241, SEQ ID NO: 242, SEQ ID NO: 243, SEQ ID NO: 244, SEQ ID NO: 245, SEQ ID NO: 246, SEQ ID NO: 247 and SEQ ID NO: 248.

The present invention provides another hybrid peptide comprising a N- to C-terminal domain: (N-terminus)-insulin B-chain—insulin A-chain-chymotrypsin inhibitory peptide-A-chain-(C-terminus), wherein the chymotrypsin inhibitory peptide directly forms a fusion peptide within the A-chain of the insulin or the analog thereof; the B-chain of the insulin or the analog is selected from the group consisting of polypeptides with the following amino acid sequences: SEQ ID NO: 34, SEQ ID NO: 35, SEQ ID NO: 36, SEQ ID NO: 39, and SEQ ID NO: 40. The “insulin A-chain chymotrypsin inhibitory peptide-A-chain-(C-terminus)” is a polypeptide formed by fusion of the chymotrypsin inhibitory peptide within the A-chain of the insulin and is selected from the group consisting of polypeptides with the following amino acid sequence: SEQ ID NO: 65, SEQ ID NO: 66, SEQ ID NO: 67 and SEQ ID NO: 68.

In another embodiment, the hybrid peptide of the insulin or the analog thereof provided by the present invention needs to be generated through the maturation and processing of a precursor protein. The precursor protein has a N- to C-terminal domain: (N-terminus)-insulin B-chain-C-polypeptide-insulin A-chain-chymotrypsin inhibitory peptide-A-chain-(C-terminus), wherein the chymotrypsin inhibitory peptide directly forms a fusion peptide within the A-chain of the insulin or the analog thereof; the B-chain of the insulin or the analog is selected from the group consisting of polypeptides with the following amino acid sequences: SEQ ID NO: 34, SEQ ID NO: 35, SEQ ID NO: 36, SEQ ID NO: 39, and SEQ ID NO: 40. The “insulin A-chain chymotrypsin inhibitory peptide-A-chain-(C-terminus)” is a polypeptide formed by fusion of the chymotrypsin inhibitory peptide within the A-chain of insulin and is selected from the group consisting of polypeptides with the following amino acid sequence: SEQ ID NO: 65, SEQ ID NO: 66, SEQ ID NO: 67 and SEQ ID NO: 68. The C-polypeptide is selected from the group consisting of polypeptides with the following amino acid sequence: SEQ ID NO: 70 and SEQ ID NO: 71. Wherein the precursor protein needs three-step treatment including protease KEX2 digestion, tobacco etch virus (TEV) protease digestion and nickel ion-induced chemical cleavage. The treatment method can be combined in a step-by-step manner. In order to obtain the hybrid peptide of the insulin or the analog thereof which does not contain two “RR” residues at the C-terminus of the B-chain, the carboxypeptidase B can be further used for treatment. To obtain the hybrid peptide of the insulin or the analog thereof with the C-terminal amidation modification, the peptidylglycine a-amidating monooxygenase can be further used for treatment.

In one particular embodiment, the precursor protein of the insulin or analog hybrid peptide thereof is selected from the group consisting of polypeptides with the following amino acid sequences: SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257 and SEQ ID NO: 258. These precursor proteins are treated using protease KEX2 digestion, tobacco etch virus (TEV) protease digestion and nickel ion-induced chemical cleavage. In order to obtain the hybrid peptides of the insulin or the analogues without two “RR” residues at the C-terminus of the B-chain, the carboxypeptidase B can be used for further treatment. Additionally, the peptidylglycine a-amidating monooxygenase can be continuously used for treatment to obtain the hybrid peptides with C-terminal amidation modification. For example, the hybrid peptide formed by fusion of the chymotrypsin inhibitory peptide CH57 (SEQ ID NO: 4) and CH10 (SEQ ID NO: 19) within the A-chain of human insulin or analog thereof is as follows:

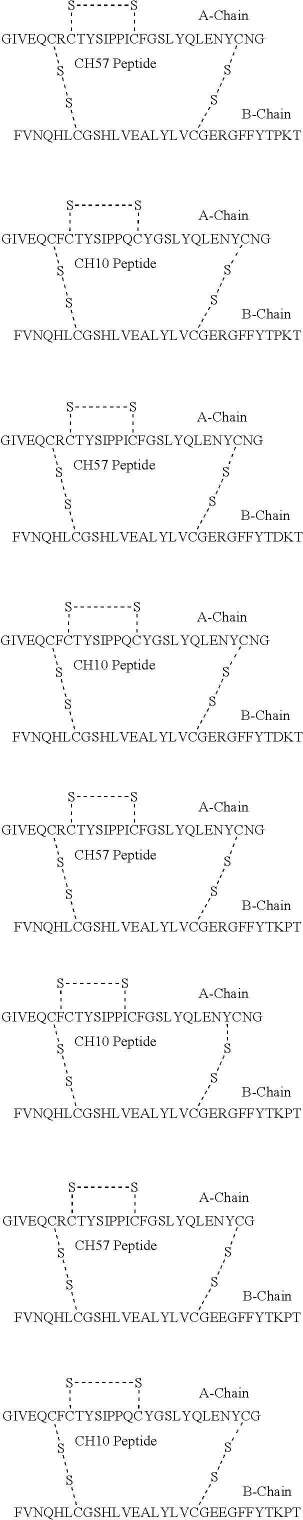

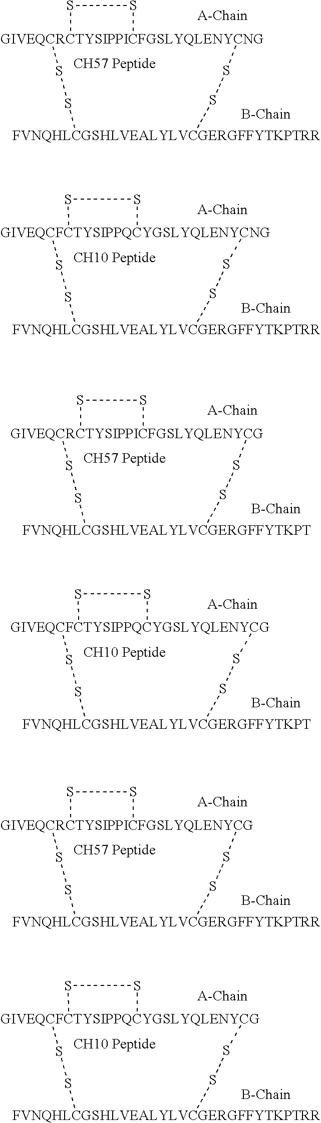

The nucleotide coding sequences of the aforementioned precursor protein of the hybrid peptide are selected from the group consisting of sequences: SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267 and SEQ ID NO: 268.

In one embodiment, the invention provides a method for preparing the hybrid peptide comprising the chymotrypsin inhibitory peptide and the insulin or the analog thereof by a heterologous organism. The biological preparation process comprises the following steps: firstly, synthesizing the precursor protein, then carrying out a series of treatment steps such as protease digestion and chemical induction cleavage, and finally carrying out chromatographic purification. The N-terminus of the precursor protein contains a cleavage site (ENLYFQ) of TEV protease as shown in SEQ ID NO: 69, and the C-peptide between the B-chain and the A-chain of the insulin molecule or the analog thereof is selected from the following the group consisting of polypeptide: SEQ ID NO: 70, SEQ ID NO: 71, SEQ ID NO: 72 and SEQ ID NO: 73. As shown in FIG. 10, in the present invention the precursor protein of the heterologous fusion expression hybrid peptide contains two cleavage sites of proteases KEX2 and TEV, wherein protease KEX2 digestion removes the expression signal peptide guiding the expression of the precursor protein to secrete, while cleaving the peptide bond after the “RR” C-terminus (the C-terminal peptide bond of the “RR” double basic amino acid residue) between the B-chain of insulin or analog and the linker peptide. TEV digests the precursor protein of the hybrid peptide to form the mature hybrid peptide; and then performing nickel ion-induced chemical cleavage, and/or treating with or without carboxypeptidase B and peptidylglycine α-amidating monooxygenase.

In one embodiment, the present invention provides the use of the hybrid peptide formed by fusion of insulin or its analogues and chymotrypsin inhibitory peptide for treating of type I and type II diabetes.

In another embodiment, the present invention provides the hybrid peptide that can be administered via subcutaneous injection and oral administration for the treatment of type I and type II diabetes.

Beneficial technical effects: the stability of insulin molecules can be improved in a mode of fusion between chymotrypsin inhibitory peptides and insulin or analogs thereof, oral administration of the insulin molecules is promoted, the medication compliance of patients can be improved, side effects can be reduced, and the beneficial economic effects are potentially achieved.

To facilitate understanding and practical application of the present invention, one or more preferred embodiments will now be described, by way of example only, with reference to the accompanying drawings.

### BRIEF DESCRIPTION OF THE DRAWINGS

The various features of the present invention have particularities in the claims. Referring to the following detailed description, a better understanding of the features and advantages of the invention will be obtained, utilizing the principles of the invention in the illustrative embodiment, the figures include:

FIG. 1. Determination of the Michaelis constant (Km) of chymotrypsin. Using Prism software, plotting the initial velocity (V₀) against the concentration of the substrate AAPFpNA yields the Michaelis constant (Km) of chymotrypsin for hydrolyzing the substrate AAPFpNA. The experiment was set up with three replicates, and the calculated values are presented as “mean±standard deviation”.

FIG. 2. Determination of the inhibitory activities of peptides against chymotrypsin. By adding different concentrations of chymotrypsin inhibitory peptides (CH5, CH11, CH18, and CH25), their inhibitory effects on chymotrypsin were investigated, and the concentrations that could produce a 50% inhibitory effect on the enzyme (IC₅₀ value) were determined. The experiment was performed with three replicates, and the calculated values were expressed as “mean±standard deviation”.

FIG. 3. Determination of inhibitory activities of chymotrypsin inhibitory peptides. By adding different concentrations of chymotrypsin inhibitory peptides (CH33, CH34, and CH35), their inhibitory effects on chymotrypsin were investigated, and the concentrations that could produce a 50% inhibitory effect on the enzyme (IC₅₀ value) were determined. The experiment was performed with three replicates, and the calculated values were expressed as “mean±standard deviation”.

FIG. 4. Determination of inhibitory activities of chymotrypsin inhibitory peptides. By adding different concentrations of chymotrypsin inhibitory peptides (CH26 and CH51), their inhibitory effects on chymotrypsin were investigated, and the concentrations that could produce a 50% inhibitory effect on the enzyme (IC₅₀ value) were determined. The experiment was performed with three replicates, and the calculated values were expressed as “mean±standard deviation”.

FIG. 5. Determination of inhibitory activities of chymotrypsin inhibitory peptides. By adding different concentrations of chymotrypsin inhibitory peptides (CH10, CH54, CH55, and CH57), their inhibitory effects on chymotrypsin were investigated, and the concentrations that could produce 50% enzyme inhibition (IC₅₀ values) were determined. The experiment was performed with three replicates, and the calculated values were expressed as “mean±standard deviation”.

FIG. 6. Determination of inhibitory activities of chymotrypsin inhibitory peptides. By adding different concentrations of chymotrypsin inhibitory peptides (CH10, CH60, CH61, and CH62), their inhibitory effects on chymotrypsin were investigated, and the concentrations that could produce a 50% inhibitory effect on the enzyme (IC₅₀ value) were determined. The experiment was performed with three replicates, and the calculated values were expressed as “mean±standard deviation”.

FIG. 7. Determination of inhibitory activities of chymotrypsin inhibitory peptides. By adding different concentrations of chymotrypsin inhibitory peptides (CH65, CH69, CH70, and CH71), their inhibitory effects on chymotrypsin were investigated, and the concentrations that could produce a 50% inhibitory effect on the enzyme (IC₅₀ value) were determined. The experiment was performed with three replicates, and the calculated values were expressed as “mean±standard deviation”.

FIG. 8. Schematic diagram of the methanol yeast expression vector pDeut-Aox1-ScKex2. It contains two promoters, AOX1 and DAS2, with different methanol-inducible expression strengths. The relatively weaker promoter DAS2 drives the expression of the protease Kex2 from Saccharomyces cerevisiae.

FIG. 9. Gene editing Cas9/gRNA expression vector targeting the AOX1 gene of methanol yeast.

FIG. 10. The expression, maturation and cleavage process of precursor proteins of insulin hybrid peptides or its analog. The αMF signal peptide guides the precursor protein to target the endoplasmic reticulum. After the signal peptide is cleaved and removed from the precursor protein in the endoplasmic reticulum, the precursor protein is transported into the trans-Golgi apparatus. The preregion of αMF and the peptide bond after the dibasic “RR” of the C-terminus of the B-chain are cleaved and removed by protease Kex2. Subsequently, the precursor protein is packaged and secreted outside the cell. After protease TEV cleavage, carboxypeptidase B cleavage, and nickel ion-induced cleavage, insulin analog hybrid peptides are obtained.

### DETAILED DESCRIPTION OF ILLUSTRATIVE EMBODIMENTS

The natural protease inhibitor is used as an additive to protect insulin molecules from proteinase degradation. Among these polypeptide protease inhibitors, an inhibitor, soybean trypsin inhibitor, selected from the Bowman-Birk protease inhibitor (BBI) family contains two active loops that inhibit proteases, inhibiting human trypsin and chymotrypsin. This protease inhibitors have been publicly described in PCT Patent WO 2014191545, WO 2019239405 and WO 2017161184.

Compared with the BBI polypeptide inhibitor, sunflower trypsin inhibitor-1 (SFTI-1) is a head-tail cyclized peptide separated from sunflower seeds, containing only 14 amino acid residues. The PCT Patent Publication No. WO 2020023386 also describes that the peptide can be used as a protease inhibitor, that is, an oral pharmaceutical component is used for treating diabetes. SFTI-1 forms a rigid structure containing two short beta-folds, an intramolecular disulfide bond, and head-tail cyclization. Although SFTI-1 has a stable cyclic structure, it is difficult to directly couple with a therapeutic polypeptide and can only be used as an additive. However, SFTI-1 only has antitrypsin activity and does not have anti-chymotrypsin activity. Therefore, SFTI-1 cannot be used as a chymotrypsin inhibitor to protect the degradation of insulin molecules against chymotrypsin.

The P1 site of serine protease inhibitory peptides determines the specificity of different serine proteases. The P1 site of chymotrypsin is amino acid residue Tyr and Phe. Only a few literatures have reported that peptide inhibitors derived from the BBI family peptide backbone exhibit anti-chymotrypsin activity [11-14], but their inhibitory activity is relatively weak. The active peptides against chymotrypsin disclosed in the literature are only used as functional applications of chymotrypsin inhibitors. Based on the core molecular backbone of the trypsin inhibitor SFTI-1, this invention replaces the P1 site to alter the protease specificity of the inhibitory peptide molecular backbone, and then replaces different recognition sites and evaluates the inhibitory activity. After a series of optimization experiments, the peptide molecular backbone against chymotrypsin was obtained as: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 11, SEQ ID NO: 12, SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28. The chymotrypsin inhibitory peptide provided by this invention directly forms a fused hybrid peptide with a bioactive peptide, which changes from merely protecting the active peptide from partial degradation by chymotrypsin at the release site as an additive against chymotrypsin, but also can accompany the active peptide, such as insulin, into the blood circulation, continuously protecting it from degradation by chymotrypsin.

The insulin molecule has good resistance to trypsin, and the most main metabolic enzyme in the small intestine metabolic enzyme is chymotrypsin which accounts for 9% of the total protein secreted by the pancreas [15]. In the present invention, the chymotrypsin inhibitory peptide is used to directly form hybrid peptides with insulin or an analog molecule thereof. The resulting formed hybrid peptide is always protected from the degradation of protease in the whole process of entering blood circulation from the microenvironment of the small intestine until the pharmacological effect is exerted.

### Peptide Synthesis

The peptide in the present invention can be prepared by various methods. For example, peptides can be synthesized through commonly used solid-phase synthesis methods, such as a-amino group t-BOC or FMOC protection method well known in the art. Here, amino acids are sequentially added to a growing chain of amino acids. The solid-phase synthesis method is particularly suitable for synthesizing peptides or relatively short peptides in large-scale production.

### Determination of Enzyme Inhibitory Activity

The inhibition constants of various synthetic active peptide inhibitors (molecular scaffolds) against serine proteases were measured. The substrates N-succinyl-Ala-Ala-Pro-Phe-p-nitroaniline (AAPFpNA) is used in competitive binding reaction to determine the inhibitory activities against α -chymotrypsin. Experiments for a-chymotrypsin was measured in 20 mM CaCl₂, 50 mM Tris-HCl buffer (pH 7.8). The concentration of the peptide was measured using optical density (OD) at 280 nm. The Michaelis constant (Km) of the enzyme hydrolysis substrate is calculated from the initial rate of substrate hydrolysis at 405 nm. The absorbance value of the substrate was measured at 405 nm after complete hydrolysis. All data were processed using nonlinear regression.

### Insulin Analogues:

The obtained chymotrypsin inhibitor can be widely used for forming insulin hybrid peptides against chymotrypsin degradation, or an analog. The stability and the in vivo and in vitro biological activity of insulin hybrid peptides are improved. The insulin or the analog thereof is not limited to the insulin or the analog thereof selected as an example disclosed in the present invention, and may be an analog having one or more amino acid residues substituted in the B-chain thereof, for example,
SEQ ID NO: 74 (FVQQHLCGSHLVEALYLVCGERGFFYTPKT),
SEQ ID NO: 75 (FVNQHLCGSHLVEALYLVCGERGFFYTPKQ),
SEQ ID NO: 76 (FVNQHLCGSHLVEALYLVCGERGFFYTPKE),
SEQ ID NO: 77 (FVQQHLCGSHLVEALYLVCGERGFFYTPKE),
SEQ ID NO: 78 (FVNQHLCGSHLVEALYLVCGERGFFYTDPT),
SEQ ID NO: 79 (FVNQHLCGSHLVQALYLVCGERGFFYTPKT),
SEQ ID NO: 80 (FVNQHLCGSHLVEALALVCGERGFFYTPKT),
SEQ ID NO: 81 (FVNQHLCGSHLVEALYLVCGEEGFFYTPKT),
SEQ ID NO: 82 (FVNQHLCGSHLVEALYLVCGEEGFFYTLPT),
SEQ ID NO: 83 (FVNQHLCGSHLVEALYLVCGERGFFYTVPT)
and SEQ ID NO: 84 (FVNQHLCGSHLVEALYLVCGERGFFYTAPT); or analogues with one or several amino acid residues replaced in the A-chain, for example,
SEQ ID NO: 85 (GIVEQCCTSICSLEQLENYCN),
SEQ ID NO: 86 (GIVEQCCTSICSLYQLENYCS),
SEQ ID NO: 87 (GIVEQCCHSICSLYQLENYCN),
SEQ ID NO: 88 (GIVEQCCWSICSLYQLENYCN)
and SEQ ID NO: 89 (GIVEQCCMSICSLYQLENYCN).

Processing and maturation of precursor proteins of insuin hybrid peptides by protease digestion:

In the present invention, hybrid peptides are expressed by heterologous secretion of the precursor proteins in eukaryotic cells, wherein the precursor proteins are cleaved by the protease Kex2 in the host cell to promote their secretion expression; then, the precursor proteins (insulin analogues) B-chain and N-terminal of A-chain (ENLYFQ, SEQ ID NO: 69) are cleaved and

modified by the TEV protease, which has a broad range of substrate characteristics [16], to release the correct B-chain or A-chain. At the same time, G↓SRHW peptide (SEQ ID NO: 72, SEQ ID NO: 73) are introduced at the C-termini of the B-chain or A-chain of the precursor proteins (insulin analogues) to form a nickel ion-induced cleavage site [17]. In addition, carboxypeptidase B can be used or not used for further processing, which can respectively obtain hybrid peptides with the C-terminus with or without two basic amino acids “RR”. When the C-terminus of B-chain or A-chain contains nickel ion induced cleavage peptide (G↓SRHW), a glycine (Gly) residue will be fused at its C-terminus after nickel ion mediated cleavage, which can be processed by recombinant amidase, so that the C-terminus of mature B-chain or A-chain forms an amidated protective modification.

C-peptide: also known as C-peptide, natural C-peptide is a single chain polypeptide composed of 31 amino acids, which connects B-chain and A-chain in proinsulin molecule. The present invention describes a peptide connecting the B-chain and the A-chain in a precursor protein of insulin hybrid peptide or its analog, which can be the same or different from the natural structure of C-peptide.

### Definition

Unless otherwise defined herein, all terms used in this application shall have the meanings that are commonly understood by those of ordinary skill in the art. The following definitions provided below were used to illustrate and clarify the description and claims of the present invention.

The singular forms “a”, “an”, and “the” include the plural, unless the context clearly indicates otherwise.

It is clear to a person skilled in the art that the polypeptide sequence disclosed herein is displayed from left to right, wherein the left end of the sequence is the N-terminal of the polypeptide, and the right end of the sequence is the C-terminal of the polypeptide.

The term “hybrid peptide” used in the present invention refers to two peptide fragments linked by a natural peptide bond, and can be prepared by solid-phase synthesis or biological expression. As used herein, the term “linker peptide” broadly refers to as a peptide fragment riching in glycine, serine or proline, capable of connecting two peptides together and forming a chemical structure. The concept of the invention is also applicable to C-polypeptide, and the function of the C-polypeptide is to connect the B-chain and the A-chain to form the precursor protein.

The term “protease inhibitor” as used in the present invention refers to as peptide molecules that inhibit the function of proteases. In one aspect, protease inhibitors inhibit the chymotrypsin found in the stomach and intestines of mammals and the proteases similar to chymotrypsin in the blood circulation.

### REFERENCES

1. Pinelo R, Roque L, Reis C P. Oral insulin delivery: utopia, currently possible or a near reality? Ther Deliv. 2021, 12 (6): 477-488.
2. Iyer G, Dyawanapelly S, Jain R, Dandekar P. An overview of oral insulin delivery strategies (OIDS). Int J Biol Macromol. 2022, 208:565-585.
3. Gedawy A, Martinez J, Al-Salami H, Dass C R. Oral insulin delivery: existing barriers and current counter-strategies. J Pharm Pharmacol. 2018, 70 (2): 197-213.
4. Twarog C, Fattah S, Heade J, et al. Intestinal permeation enhancers for oral delivery of macromolecules: a comparison between salcaprozate sodium (SNAC) and sodium caprate (C10). Pharmaceutics 11, E78 (2019).
5. Liu H, Tang R, Pan W S, et al. Potential utility of various protease inhibitors for improving the intestinal absorption of insulin in rats. J Pharm Pharmacol, 2003, 55 (11): 1523-1529.
6. Drucker D J. Advances in oral peptide therapeutics. Nat Rev Drug Discov, 2020, 19 (4): 277-289.
7. Zijlstra E, Heinemann L, Plum-Morschel L. Oral insulin reloaded: a structured approach. J Diabetes Sci Technol, 2014, 8 (3): 458-465.
8. Eldor R, Arbit E, Corcos A, Kidron, M. Glucose-reducing effect of the ORMD-0801 oral insulin preparation in patients with uncontrolled type 1 diabetes: a pilot study. PLOS ONE,2013, 8: e59524.
9. Zhang Y, Zhou W, Shen L, et al. Safety, Pharmacokinetics, and Pharmacodynamics of Oral Insulin Administration in Healthy Subjects: A Randomized, Double-Blind, Phase 1 Trial. Clin Pharmacol Drug Dev. 2022, 11 (5): 606-614.
10. Schilling R J, Mitra A K. Degradation of insulin by trypsin and alpha-chymotrypsin. Pharm Res. 1991, 8 (6): 721-727.
11. McBride J D, Brauer A B, Nievo M, Leatherbarrow R J. The role of threonine in the P2 position of Bowman-Birk proteinase inhibitors: studies on P2 variation in cyclic peptides encompassing the reactive site loop. J Mol Biol, 1998, 282:447-458.
12. McBride J D, Freeman N, Domingo G J, Leatherbarrow R J. Selection of chymotrypsin inhibitors from a conformationally-constrained combinatorial peptide library. J Mol Biol, 1996, 259:819-827.
13. McBride J D, Freeman H N, Leatherbarrow R J. Identification of chymotrypsin inhibitors from a second-generation template assisted combinatorial peptide library. J Pept Sci, 2000, 6:446-452.
14. Begowska A, Debowski D, Lesner A, Wysocka M, Rolka K. Introduction of non-natural amino acid residues into the substrate-specific P1 position of trypsin inhibitor SFTI-1 yields potent chymotrypsin and cathepsin G inhibitors. Bioorg Med Chem, 2009, 17 (9): 3302-3307.
15. Whitcomb D C, Lowe M E. Human pancreatic digestive enzymes. Dig Dis Sci, 2007, 52 (1): 1-17.
16. Kapust R B, Tözsér J, Copeland T D, Waugh D S. The P1′ specificity of tobacco etch virus protease. Biochem Biophys Res Commun, 2002, 294 (5): 949-55.
17. Dang B, Mravic M, Hu H, et al. SNAC-tag for sequence-specific chemical protein cleavage. Nat Methods, 2019, 16 (4): 319-322.

The example below is provided to illustrate the embodiments in the present invention and is intended only for a better understanding of the present invention, but is not be interpreted as limiting the scope or spirit of the invention.

### EXAMPLES

### Example 1 Solid-phase Synthesis of Peptides

According to the sequence of the amino acid residues of each polypeptide, the polypeptide is synthesized from C-terminal to N-terminal one by one through the solid-phase chemical synthesis method using Fmoc (9-fluorenylmethoxycarbonyl) amino protective agents; when the synthesis of the linear peptide protected by the side chain of the amino acid is completed, the linear peptide is cleaved from the resin, the protective group of amino acid residues in the linear peptide is removed, then the intramolecular sulfhydryl group is oxidized to form the disulfide bonds, finally, the target polypeptide is obtained by the purification of HPLC reversed-phase C18 column chromatography.

### I. Materials

(1) Resins: Fmoc-Ala-Wang resin, Fmoc-Arg(Pbf)-Wang resin, Fmoc-Asn(Trt)-Wang resin, Fmoc-Asp (OtBu)-Wang resin, Fmoc-Gly-Wang resin, Fmoc-Phe-Wang resin, Fmoc-Pro-Wang resin, Fmoc-Tyr (tBu)-Wang resin.

(2) Reagents: piperidine, DMF, DCM, 4-Picoline, DIEA, HATU, HOBT, TBTU, DIC, TFA, EDT, TIPS, TA, phenol, diethyl ether, DMSO, distilled water.

### II. Synthesis Method

SEQ ID NO: 1
(Gly-Arg-Cys-Thr-Phe-Ser-Ile-Pro-Pro-Ile-Cys-Phe-
Pro-Asp)

(1) Weighed Fmoc-Asp (OtBu)-Wang resin into the glass reaction column and added DCM to swell for 30 min, and then the DCM was removed by vacuum filtration.

(2) Washed the resin with DMF for three times, added piperidine/DMF (1:4, v/v) solution to react for 20 min to remove the protecting group Fmoc. The solution was removed by vacuum filtration, then washed the resin with DMF for six times.

(3) Weighed Fmoc-Pro-OH and TBTU, and added them into the resin and dissolved by DMF. Added DIEA to react for 30 min, and took out of the resin to perform Kaiser Test. It was proved that the reaction was completed when the solution became bright yellow and the resin became yellow. The solvent could be removed by vacuum filtration.

(1) Repeated steps 2 and 3 and then obtained the peptide segment with the protecting group, namely Fmoc-Gly-Arg(Pbf)-Cys(Trt)-Thr(tBu)-Phe-Ser(tBu)-Ile-Pro-Pro-Ile-Cys(Trt)-Phe-Pro-Asp (OtBu)-Wang resin. Removed the Fmoc, washed the resin with DMF, DCM and methanol for three times, respectively, and then drained the resin by vacuum filtration.

(5) The resin and the side-chain protecting group were removed by treatment with cleavage reagent (TFA, EDT, TA, phenol and distilled water mixed in certain proportion). Filtered with gravel core, added ether into the filtrate for precipitation, centrifuged and washed the solid for three times, drained by vacuum filtration.

(6) Dissolved with H₂O/acetonitrile (9:1, v/v), and the volume was increased to 100 mL. Added dilute ammonia solution to adjust pH to basic (pH~ 8) and the sample was taken out to test the activity of the thiol group. It indicated the presence of the thiol group when the solution turned yellow. The oxidation was completed (more than 90%) when the solution became clear after addition of 2-3 drops of hydrogen peroxide to react for 5-10 min. Added glacial acetic acid to adjust pH to acidic (pH~ 6), and the chemical structure of the peptide was characterized by mass spectrometry. The target peptide of the correct molecular weight was obtained by purification using reversed-phase HPLC on a C18 column.

(7) The measured molecular weight of peptide SEQ ID NO: 1 (CH54) is 1550.4 Da ([M+2H²⁺=776.2).

SEQ ID NO: 2
(Gly-Arg-Cys-Thr-Tyr-Ser-Ile-Pro-Pro-Ile-Cys-Phe-
Pro-Asp)

Fmoc-Asp (OtBu)-Wang resin was selected as the starting material of chemical synthesis of peptide SEQ ID NO: 2, which was synthesized according to the method described in chemical synthesis of peptide SEQ ID NO: 1. At first, the amino acids were added successively to synthesize the peptide segment with the protecting groups, namely Fmoc-Gly-Arg(Pbf)-Cys(Trt)-Thr(tBu)-Tyr (tBu)-Ser(tBu)-Ile-Pro-Pro-Ile-Cys(Trt)-Phe-Pro-Asp (OtBu)-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 2 (CH55) was obtained by separation and purification with the measured molecular weight of 1566.4 Da ([M+2H]²⁺=784.2).

SEQ ID NO: 3
(Gly-Arg-Cys-Thr-Tyr-Ser-Ile-Pro-Pro-Ile-Cys-Phe-
Pro)

Fmoc-Pro-Wang resin was selected as the starting material of chemical synthesis of peptide SEQ ID NO: 3 which was synthesized according to the method described in chemical synthesis of peptide SEQ ID NO: 1. At first, the amino acids were added successively to synthesize the peptide segment with the protecting groups, namely Fmoc-Gly-Arg(Pbf)-Cys(Trt)-Thr(tBu)-Tyr (tBu)-Ser(tBu)-Ile-Pro-Pro-Ile-Cys(Trt)-Phe-Pro-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 3 (CH56) was obtained by separation and purification with the measured molecular weight of 1452.2 Da ([M+2H]²⁺=727.1).

SEQ ID NO: 4
(Gly-Arg-Cys-Thr-Tyr-Ser-Ile-Pro-Pro-Ile-Cys-Phe)

Fmoc-Phe-Wang resin was selected as the starting material of chemical synthesis of peptide SEQ ID NO: 4 which was synthesized according to the method described in chemical synthesis of peptide SEQ ID NO: 1. At first, the amino acids were added successively to synthesize the peptide segment with the protecting groups, namely Fmoc-Gly-Arg(Pbf)-Cys(Trt)-Thr(tBu)-Tyr (tBu)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 4 (CH57) was obtained by separation and purification with the measured molecular weight of 1354.2 Da ([M+2H]²⁺=678.1).

SEQ ID NO: 5
(Arg-Cys-Thr-Tyr-Ser-Ile-Pro-Pro-Ile-Cys-Phe)

Fmoc-Phe-Wang resin was selected as the starting material of chemical synthesis of peptide SEQ ID NO: 5 which was synthesized according to the method described in chemical synthesis of peptide SEQ ID NO: 1. At first, the amino acids were added successively to synthesize the peptide segment with the protecting groups, namely Fmoc-Arg(Pbf)-Cys(Trt)-Thr(tBu)-Tyr (tBu)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 5 (CH58) was obtained by separation and purification with the measured molecular weight of 1297.0 Da ([M+2H]²⁺=649.5).

SEQ ID NO: 6
(Gly-Thr-Cys-Thr-Phe-Ser-Ile-Pro-Pro-Ile-Cys-Asn-
Pro-Asn)

Fmoc-Asn(Trt)-Wang resin was selected as the starting material of chemical synthesis of peptide SEQ ID NO: 6 which was synthesized according to the method described in chemical synthesis of peptide SEQ ID NO: 1. At first, the amino acids were added successively to synthesize the peptide segment with the protecting groups, namely Fmoc-Gly-Thr(tBu)-Cys(Trt)-Thr(tBu)-Phe-Ser(tBu)-Ile-Pro-Pro-Ile-Cys(Trt)-Asn-Pro-Asn(Trt)-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 6 (CH59) was obtained by separation and purification with the measured molecular weight of 1461.0 Da ([M+2H]²⁺=731.5).

SEQ ID NO: 7
(Gly-Thr-Cys-Thr-Tyr-Ser-Ile-Pro-Pro-Ile-Cys-Asn-
Pro-Asn)

Fmoc-Asn(Trt)-Wang resin was selected as the starting material of chemical synthesis of peptide SEQ ID NO: 7 which was synthesized according to the method described in chemical synthesis of peptide SEQ ID NO: 1. At first, the amino acids were added successively to synthesize the peptide segment with the protecting groups, namely Fmoc-Gly-Thr(tBu)-Cys(Trt)-Thr(tBu)-Tyr (tBu)-Ser(tBu)-Ile-Pro-Pro-Ile-Cys(Trt)-Asn(Trt)-Pro-Asn(Trt)-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 7 (CH60) was obtained by separation and purification with the measured molecular weight of 1477.2 Da ([M+2H]²⁺=739.6).

SEQ ID NO: 8
(Gly-Thr-Cys-Thr-Tyr-Ser-Ile-Pro-Pro-Ile-Cys-Asn-
Pro)

Fmoc-Pro-Wang resin was selected as the starting material of chemical synthesis of peptide SEQ ID NO: 8 which was synthesized according to the method described in chemical synthesis of peptide SEQ ID NO: 1. At first, the amino acids were added successively to synthesize the peptide segment with the protecting groups, namely Fmoc-Gly-Thr(tBu)-Cys(Trt)-Thr(tBu)-Tyr (tBu)-Ser(tBu)-Ile-Pro-Pro-Ile-Cys(Trt)-Asn(Trt)-Pro-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 8 (CH61) was obtained by separation and purification with the measured molecular weight of 1363.2 Da ([M+2H]²⁺=682.6).

SEQ ID NO: 9
(Gly-Thr-Cys-Thr-Tyr-Ser-Ile-Pro-Pro-Ile-Cys-Asn)

Fmoc-Asn(Trt)-Wang resin was selected as the starting material of chemical synthesis of peptide SEQ ID NO: 9 which was synthesized according to the method described in chemical synthesis of peptide SEQ ID NO: 1. At first, the amino acids were added successively to synthesize the peptide segment with the protecting groups, namely Fmoc-Gly-Thr(tBu)-Cys(Trt)-Thr(tBu)-Tyr (tBu)-Ser(tBu)-Ile-Pro-Pro-Ile-Cys(Trt)-Asn(Trt)-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 9 (CH62) was obtained by separation and purification with the measured molecular weight of 1266.0 Da ([M+2H]²⁺=634.0).

SEQ ID NO: 10
(Thr-Cys-Thr-Tyr-Ser-Ile-Pro-Pro-Ile-Cys-Asn)

Fmoc-Asn(Trt)-Wang resin was selected as the starting material of chemical synthesis of peptide SEQ ID NO: 10 which was synthesized according to the method described in chemical synthesis of peptide SEQ ID NO: 1. At first, the amino acids were added successively to synthesize the peptide segment with the protecting groups, namely Fmoc-Thr(tBu)-Cys(Trt)-Thr(tBu)-Tyr (tBu)-Ser(tBu)-Ile-Pro-Pro-Ile-Cys(Trt)-Asn(Trt)-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 10 (CH63) was obtained by separation and purification with the measured molecular weight of 1209.3 Da ([M−H]⁻=1208.3).

SEQ ID NO: 11
(Lys-Cys-Thr-Tyr-Ser-Ile-Pro-Pro-Ile-Cys-Phe)

Fmoc-Phe-Wang resin was selected as the starting material of chemical synthesis of peptide SEQ ID NO: 11 which was synthesized according to the method described in chemical synthesis of peptide SEQ ID NO: 1. At first, the amino acids were added successively to synthesize the peptide segment with the protecting groups, namely Fmoc-Lys (Boc)-Cys(Trt)-Thr(tBu)-Tyr (tBu)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 11 (CH64) was obtained by separation and purification with the measured molecular weight of 1269.2 Da ([M+2H]²⁺=635.6).

SEQ ID NO: 12
(Cys-Gly-Arg-Ala-Thr-Tyr-Ser-Ile-Pro-Pro-Ile-Cys-Phe-Pro-Asp)

Fmoc-Asp (OtBu)-Wang resin was selected as the starting material of chemical synthesis of peptide SEQ ID NO: 12 which was synthesized according to the method described in chemical synthesis of peptide SEQ ID NO: 1. At first, the amino acids were added successively to synthesize the peptide segment with the protecting groups, namely Fmoc-Cys(Trt)-Gly-Arg(Pbf)-Ala-Thr(tBu)-Tyr (tBu)-Ser(tBu)-Ile-Pro-Pro-Ile-Cys(Trt)-Phe-Pro-Asp (OtBu)-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 12 (CH65) was obtained by separation and purification with the measured molecular weight of 1637.2 Da ([M+2H]²⁺=819.6).

SEQ ID NO: 13
(Cys-Gly-Arg-Ala-Thr-Tyr-Ser-Ile-Pro-Pro-Ile-Cys-Phe-Pro)

Fmoc-Pro-Wang resin was selected as the starting material of chemical synthesis of peptide SEQ ID NO: 13 which was synthesized according to the method described in chemical synthesis of peptide SEQ ID NO: 1. At first, the amino acids were added successively to synthesize the peptide segment with the protecting groups, namely Fmoc-Cys(Trt)-Gly-Arg(Pbf)-Ala-Thr(tBu)-Tyr (tBu)-Ser(tBu)-Ile-Pro-Pro-Ile-Cys(Trt)-Phe-Pro-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 13 (CH66) was obtained by separation and purification with the measured molecular weight of 1523.2 Da ([M+2H]²⁺=762.6).

SEQ ID NO: 14
(Cys-Gly-Arg-Ala-Thr-Tyr-Ser-Ile-Pro-Pro-Ile-Cys-
Phe)

Fmoc-Phe-Wang resin was selected as the starting material of chemical synthesis of peptide SEQ ID NO: 14 which was synthesized according to the method described in chemical synthesis of peptide SEQ ID NO: 1. At first, the amino acids were added successively to synthesize the peptide segment with the protecting groups, namely Fmoc-Cys(Trt)-Gly-Arg(Pbf)-Ala-Thr(tBu)-Tyr (tBu)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 14 (CH67) was obtained by separation and purification with the measured molecular weight of 1425.2 Da ([M+2H]²⁺=713.6).

SEQ ID NO: 15
(Cys-Gly-Arg-Ala-Abu-Thr-Tyr-Ser-Ile-Pro-Pro-Ile-
Cys-Phe)

Fmoc-Phe-Wang resin was selected as the starting material of chemical synthesis of peptide SEQ ID NO: 15 which was synthesized according to the method described in chemical synthesis of peptide SEQ ID NO: 1. At first, the amino acids were added successively to synthesize the peptide segment with the protecting groups, namely Fmoc-Cys(Trt)-Gly-Arg(Pbf)-Ala-Abu-Thr(tBu)-Tyr (tBu)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 15 (CH68) was obtained by separation and purification with the measured molecular weight of 1439.2 Da ([M+2H]²⁺=720.6).

SEQ ID NO: 16
(Cys-Lys-Arg-Ala-Thr-Tyr-Ser-Ile-Pro-Pro-Ile-Cys-
Phe)

Fmoc-Phe-Wang resin was selected as the starting material of chemical synthesis of peptide SEQ ID NO: 16 which was synthesized according to the method described in chemical synthesis of peptide SEQ ID NO: 1. At first, the amino acids were added successively to synthesize the peptide segment with the protecting groups, namely Fmoc-Cys(Trt)-Lys (Boc)-Arg(Pbf)-Ala-Thr(tBu)-Tyr (tBu)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 16 (CH69) was obtained by separation and purification with the measured molecular weight of 1496.2 Da ([M+2H]²⁺=749.1).

SEQ ID NO: 17
(Cys-Glu-Arg-Ala-Thr-Tyr-Ser-Ile-Pro-Pro-Ile-Cys-
Phe)

Fmoc-Phe-Wang resin was selected as the starting material of chemical synthesis of peptide SEQ ID NO: 17 which was synthesized according to the method described in chemical synthesis of peptide SEQ ID NO: 1. At first, the amino acids were added successively to synthesize the peptide segment with the protecting groups, namely Fmoc-Cys(Trt)-Glu (OtBu)-Arg(Pbf)-Ala-Thr(tBu)-Tyr (tBu)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 17 (CH70) was obtained by separation and purification with the measured molecular weight of 1497.2 Da ([M+2H]²⁺=749.6).

SEQ ID NO: 18
(Cys-Gly-Arg-Ala-Thr-Phe-Ser-Ile-Pro-Pro-Ile-Cys-
Phe)

Fmoc-Phe-Wang resin was selected as the starting material of chemical synthesis of peptide SEQ ID NO: 18 which was synthesized according to the method described in chemical synthesis of peptide SEQ ID NO: 1. At first, the amino acids were added successively to synthesize the peptide segment with the protecting groups, namely Fmoc-Cys(Trt)-Gly-Arg(Pbf)-Ala-Thr(tBu)-Phe-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 18 (CH71) was obtained by separation and purification with the measured molecular weight of 1409.2 Da ([M+2H]²⁺=705.6).

SEQ ID NO: 19
(Phe-Cys-Thr-Tyr-Ser-Ile-Pro-Pro-Gln-Cys-Tyr-Gly)

Fmoc-Gly-Wang resin was selected as the starting material of chemical synthesis of peptide SEQ ID NO: 19 which was synthesized according to the method described in chemical synthesis of peptide SEQ ID NO: 1. At first, the amino acids were added successively to synthesize the peptide segment with the protecting groups, namely Fmoc-Phe-Cys(Trt)-Thr(tBu)-Tyr (tBu)-Ser(tBu)-Ile-Pro-Pro-Gln (Trt)-Cys(Trt)-Tyr (tBu)-Gly-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 19 (CH10) was obtained by separation and purification with the measured molecular weight of 1375.55 Da ([M+Na]+=1398.55).

SEQ ID NO: 20
(Phe-Cys-Thr-Phe-Ser-Ile-Pro-Pro-Gln-Cys-Tyr-Gly)

Fmoc-Gly-Wang resin was selected as the starting material of chemical synthesis of peptide SEQ ID NO: 20 which was synthesized according to the method described in chemical synthesis of peptide SEQ ID NO: 1. At first, the amino acids were added successively to synthesize the peptide segment with the protecting groups, namely Fmoc-Phe-Cys(Trt)-Thr(tBu)-Phe-Ser(tBu)-Ile-Pro-Pro-Gln (Trt)-Cys(Trt)-Tyr (tBu)-Gly-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 20 (CH5) was obtained by separation and purification with the measured molecular weight of 1360.02 Da ([M+k+H]²⁺=700.01).

SEQ ID NO: 21
(Ser-Cys-Thr-Phe-Ser-Ile-Pro-Pro-Gln-Cys-Tyr-Gly)

Fmoc-Gly-Wang resin was selected as the starting material of chemical synthesis of peptide SEQ ID NO: 21 which was synthesized according to the method described in chemical synthesis of peptide SEQ ID NO: 1. At first, the amino acids were added successively to synthesize the peptide segment with the protecting groups, namely Fmoc-Ser(tBu)-Cys(Trt)-Thr(tBu)-Phe-Ser(tBu)-Ile-Pro-Pro-Gln (Trt)-Cys(Trt)-Tyr (tBu)-Gly-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 21 (CH11) was obtained by separation and purification with the measured molecular weight of 1300.55 Da ([M+H]⁺).

SEQ ID NO: 22
(Ala-Cys-Thr-Tyr-Ser-Ile-Pro-Ala-Lys-Cys-Phe)

Fmoc-Phe-Wang resin was selected as the starting material of chemical synthesis of peptide SEQ ID NO: 22 which was synthesized according to the method described in chemical synthesis of peptide SEQ ID NO: 1. At first, the amino acids were added successively to synthesize the peptide segment with the protecting groups, namely Fmoc-Ala-Cys(Trt)-Thr(tBu)-Tyr (tBu)-Ser(tBu)-Ile-Pro-Ala-Lys (Boc)-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 22 (CH18) was obtained by separation and purification with the measured molecular weight of 1200.80 Da ([M+2H]²⁺=601.40).

SEQ ID NO: 23
(Gly-Thr-Cys-Thr-Phe-Ser-Ile-Pro-Pro-Ile-Cys-Asn-
Pro-Asn)

Fmoc-Asn(Trt)-Wang resin was selected as the starting material of chemical synthesis of peptide SEQ ID NO: 23 which was synthesized according to the method described in chemical synthesis of peptide SEQ ID NO: 1. At first, the amino acids were added successively to synthesize the peptide segment with the protecting groups, namely Fmoc-Gly-Thr(tBu)-Cys(Trt)-Thr(tBu)-Phe-Ser(tBu)-Ile-Pro-Pro-Ile-Cys(Trt)-Asn(Trt)-Pro-Asn(Trt)-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 23 (CH25) was obtained by separation and purification with the measured molecular weight of 1461.00 Da ([M+2H]²⁺=731.50).

SEQ ID NO: 24
(Gly-Thr-Cys-Thr-Phe-Ser-Ile-Pro-Pro-Ile-Cys-Asn)

Fmoc-Asn(Trt)-Wang resin was selected as the starting material of chemical synthesis of peptide SEQ ID NO: 24 which was synthesized according to the method described in chemical synthesis of peptide SEQ ID NO: 1. At first, the amino acids were added successively to synthesize the peptide segment with the protecting groups, namely Fmoc-Gly-Thr(tBu)-Cys(Trt)-Thr(tBu)-Phe-Ser(tBu)-Ile-Pro-Pro-Ile-Cys(Trt)-Asn(Trt)-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 24 (CH26) was obtained by separation and purification with the measured molecular weight of 1249.50 Da ([M+Na]+=1272.50).

SEQ ID NO: 25
(Phe-Cys-Thr-Tyr-Ser-Ile-Pro-Pro-Gln-Cys-Tyr)

Fmoc-Tyr (tBu)-Wang resin was selected as the starting material of chemical synthesis of peptide SEQ ID NO: 25 which was synthesized according to the method described in chemical synthesis of peptide SEQ ID NO: 1. At first, the amino acids were added successively to synthesize the peptide segment with the protecting groups, namely Fmoc-Phe-Cys(Trt)-Thr(tBu)-Tyr (tBu)-Ser(tBu)-Ile-Pro-Pro-Gln (Trt)-Cys(Trt)-Tyr (tBu)-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 25 (CH33) was obtained by separation and purification with the measured molecular weight of 1318.80 Da ([M+2H]²⁺=660.40).

SEQ ID NO: 26
(Phe-Cys-Thr-Tyr-Ser-Ile-Pro-Pro-Gln-Cys-Tyr-Ala)

Fmoc-Ala-Wang resin was selected as the starting material of chemical synthesis of peptide SEQ ID NO: 26 which was synthesized according to the method described in chemical synthesis of peptide SEQ ID NO: 1. At first, the amino acids were added successively to synthesize the peptide segment with the protecting groups, namely Fmoc-Phe-Cys(Trt)-Thr(tBu)-Tyr (tBu)-Ser(tBu)-Ile-Pro-Pro-Gln (Trt)-Cys(Trt)-Tyr (tBu)-Ala-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 26 (CH34) was obtained by separation and purification with the measured molecular weight of 1390.80 Da ([M+2H]²⁺=696.40).

SEQ ID NO: 27
(Phe-Cys-Thr-Tyr-Ser-Ile-Pro-Pro-Gln-Cys-Arg)

Fmoc-Arg(Pbf)-Wang resin was selected as the starting material of chemical synthesis of peptide SEQ ID NO: 27 which was synthesized according to the method described in chemical synthesis of peptide SEQ ID NO: 1. At first, the amino acids were added successively to synthesize the peptide segment with the protecting groups, namely Fmoc-Phe-Cys(Trt)-Thr(tBu)-Tyr (tBu)-Ser(tBu)-Ile-Pro-Pro-Gln (Trt)-Cys(Trt)-Arg(Pbf)-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 27 (CH35) was obtained by separation and purification with the measured molecular weight of 1312.20 Da ([M+2H]²⁺=657.10).

SEQ ID NO: 28
(Pro-Cys-Thr-Tyr-Ser-Ile-Pro-Pro-Gln-Cys-Tyr)

Fmoc-Tyr (tBu)-Wang resin was selected as the starting material of chemical synthesis of peptide SEQ ID NO: 28 which was synthesized according to the method described in chemical synthesis of peptide SEQ ID NO: 1. At first, the amino acids were added successively to synthesize the peptide segment with the protecting groups, namely Fmoc-Pro-Cys(Trt)-Thr(tBu)-Tyr (tBu)-Ser(tBu)-Ile-Pro-Pro-Gln (Trt)-Cys(Trt)-Tyr (tBu)-Wang resin. Then the Fmoc was removed, and the resin and the side-chain protecting groups were also removed by addition of the cleavage buffer, followed by formation of the disulfide bond via oxidation. Finally, the target peptide segment SEQ ID NO: 28 (CH51) was obtained by separation and purification with the measured molecular weight of 1268.80 Da ([M+2H]²⁺=635.40).

### Example 2

### Design and Inhibitory Activity Evaluation of Peptides Against Chymotrypsin

### Determination of the Michaelis Constant Kₘ Value:

(1) Added a total 198 μL of reaction buffer (20 mM CaCl₂, 50 mM Tris-HCl (pH 7.8)) into the 96 well plate, followed by preheating at 37° C. for 15 min. Then added 2 μL of different concentrations of substrates (p-Nitroanilide, pNA, 0.5% in DMSO), and mixed by centrifugation at 500 rpm for 1 min. Finally, the plate was incubated at 37° C. for 20 min, and the absorbance value at 405 nm was measured. In the 200 μL of reaction system, the final concentrations of pNA were 0, 0.025, 0.05, 0.075, 0.1, 0.125, 0.15, 0.25, and 0.3 mM, respectively. Make three repetitions for each concentration and plot the OD 405 nm value with pNA concentration to obtain the standard curve. (2) Added a total 190 μL of reaction buffer (20 mM CaCl₂, 50 mM Tris-HCl (pH 7.8)) and 8 μL of 0.75 μM chymotrypsin into the 96 well plate, followed by preheating at 37° C. for 5 min. Then added 2 μL of different concentrations of substrates (AAPFpNA, dissolved in DMSO), and mixed by centrifugation at 500 rpm for 1 min. Finally, the plate was incubated at 37° C. for 20 min, and the absorbance value at 405 nm was measured. In the 200 μL of reaction system, the final concentrations of AAPFpNA were 0, 0.125, 0.25, 0.285, 0.33, 0.4, and 0.5 mM, respectively. Make three repetitions for each concentration and plot the OD 405 nm value with time to obtain the corresponding curve. Divide the slope of the curve by the slope of the standard curve and the enzyme concentration to obtain the initial velocity V₀ (mM/(min*mM protein). Plot the initial velocity V₀ with the concentration of substrate AAPFpNA using Prism software, and the Michaelis constant Kₘ value of chymotrypsin hydrolyzing AAPFpNA was obtained.

### Determination of the Inhibition Constant Kᵢ Value:

(1) Added a total 190 μL of reaction buffer (20 mM CaCl₂, 50 mM Tris-HCl (pH 7.8)), different concentrations of anti-chymorypsin peptides (CHs) into the pre-cold 96 well plate, followed by preheating at 37° C. for 5 min and centrifugated at 500 rpm for 1 min and allowed to stand for 4 minutes. Then added 8 μL of 750 nM chymotrypsin and incubated at 37° C. for 10 min, and centrifugated at 500 rpm for 1 min and allowed to stand for 9 minutes. Finally, added 2 μL of 50 mM AAPFpNA, and mixed by centrifugation at 500 rpm for 1 min. The plate was incubated at 37° C. for 90 min, and the absorbance value at 405 nm was measured. Three repetitions were made, and only reaction buffer and substrate were added in the blank control as the minimum absorption value (Min OD 405 nm); Only reaction buffer, enzyme and substrate were added in the negative control as the maximum absorption value (Max OD 405 nm).

(2) In the 200 μL of reaction system, the final concentrations of chymotrypsin and AAPFpNA were 30 nM and 0.5 mM, respectively.

### (3) Data Statistics

$$Residual activity of enzyme ( \% ) = ( 1 - ( Max {OD}_{405 nm} - Sample {OD}_{405 nm} ) / ( Max {OD}_{405 nm} - Min {OD}_{405 nm} ) ) * 100$$

Plotted the residual activity of enzyme with substrate concentration to obtain the half inhibitory concentration (IC50) of anti-chymorypsin peptides (CHs). Then, substituted it into the formula Kᵢ+IC50/(1+S/Kₘ) (S, IC50, and Kₘ were substrate concentration, half inhibitory concentration, and Michaelis constant, respectively) to obtain the inhibition constant Kᵢ of anti-chymorypsin peptides (CHs).

### Results:

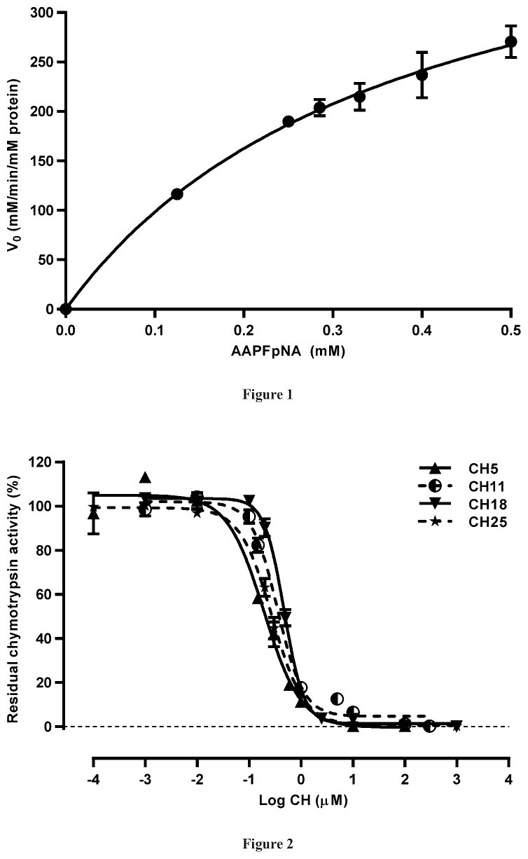

Using a certain concentration of chymotrypsin to catalyze different concentrations of AAPFpNA to produce pNA, and the absorbance value of OD 405 nm was measured. Referring to the standard curve, Prism software was used to plot the initial velocity V₀ with the concentration of substrate AAPFpNA, and the Michaelis constant Kₘ value of chymotrypsin hydrolyzing AAPFpNA was obtained to be 0.38 mM (R²=0.9988) (FIG. 1).

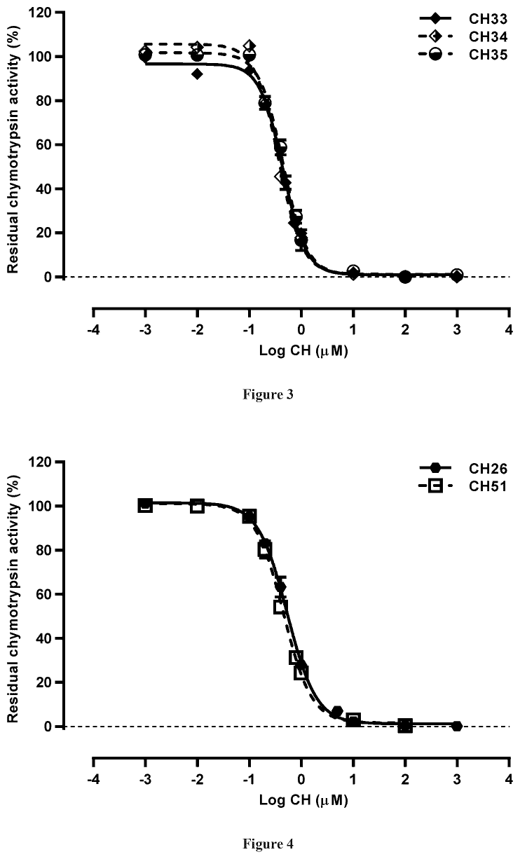

Most of the protease inhibitors of the natural Bowman-Birk Inhibitor (BBI) family contain two independent inhibitory sites which respectively inhibit trypsin and chymotrypsin; the soybean BBI inhibitor is composed of 71 amino acid residues and is cross-linked by seven disulfide bonds, one of the sites for inhibiting trypsin is located at Lys16-Ser17, the site of the other inhibiting chymotrypsin is located at Leu43-Ser44. It forms a 1:1 ternary complex with trypsin or chymotrypsin. This natural structure of BBI has weak inhibitory activity against proteases. It is reported that cyclic peptides linked by disulfide bonds can be obtained with a single inhibitory activity by simplifying the BBI structure [McBride J D, Freeman N, Domingo G J, Leatherbarrow R J. Selection of chymotrypsin inhibitors from a conformationally-constrained combinatorial peptide library. J Mol Biol, 1996, 259:819-827.]. In combination with the specificity of chymotrypsin P1 sites (specifically recognizing Tyr and Phe) and the conservative properties of P1′ and P3′, a series of polypeptides (Table 1) cyclized by disulfide bonds and having a core structure of “CTYSIPPQC” are designed and synthesized in the present invention, two polypeptides CH10 and CH5 with high inhibitory activity are obtained through in vitro inhibitory activity determination, the IC50 values of the polypeptides CH10 and CH5 are 0.06 μM and 0.19 μM respectively (table 2), and the results prove that the inhibition activity of P1 sites on chymotrypsin has a significant impact on the inhibitory activity of chymotrypsin. Then, CH11, CH35 and CH51 are obtained by replacing P4 and P7′ amino acid residues on the basis of CH10, the inhibitory activity of the CH11, CH35 and CH51 is reduced by nearly 7 times, and it is indicated that the amino acid residues of P4 and P7′ can enhance the intermolecular acting force with the active sites of chymotrypsin (table 2). By comparing the inhibitory activity differences of CH33, CH34 and CH10, it can be figured out that the P8′ glycine with relatively strong polarity is more beneficial to enhancing the intermolecular interaction between the P8 ‘glycine and the protease (table 2). The extension (CH25, CH26) of the P5 site and the amino acid substitution (CH18) of the P4 ‘site have no large change to the inhibition activity (table 2, and FIGS. 2, 3 and 4)

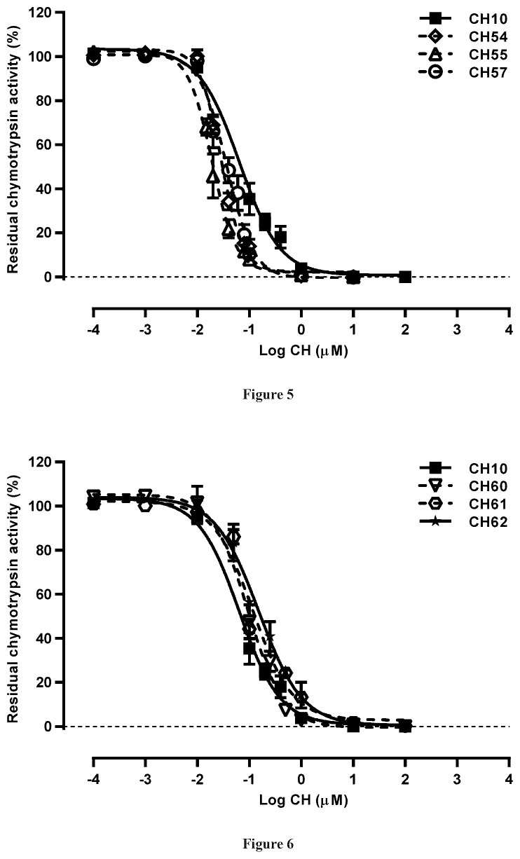

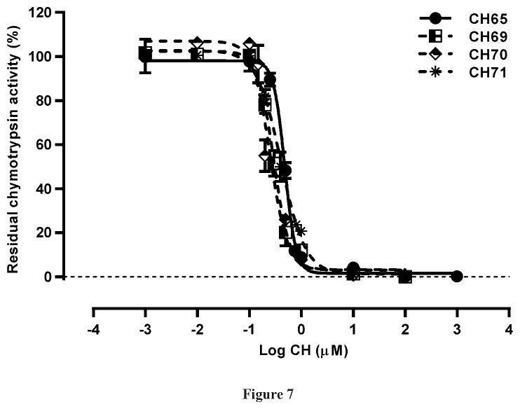

A protease inhibitor SFTI-1 (GRCTKSIPPICFPD) relative to other BBI families has a special structure and only contains a pair of disulfide bonds with a head-tail cyclization structure. According to the specificity of the P1 site of the chymotrypsin P1, Lys at the P1 site is directly mutated to Tyr or Phe, the polypeptide CH54 and CH55 (table 1) are designed and synthesized, both have good chymotrypsin inhibition activity, and their IC50 value is 0.03 μM and 0.02 μM (FIG. 5). In order to further simplify the structure thereof to synthesize CH56, CH57 and CH58 (table 1), the inhibition of the three can show that the Pro at the P8’ position can generate steric hindrance and reduce the inhibition effect thereof, and the IC50 (0.62 μM) thereof is reduced by 30 times relative to CH55 (IC₅₀=0.02 μM) (table 2). At the same time, the inhibition activity of CH57 and CH58 shows that the Gly at the P5 site is beneficial to enhancing the interaction between the inhibitory peptide and the active central region of chymotrypsin, and the amino acid residue at the P5 site has a great influence on the inhibitory activity; and the result also indicates that CH58 is a core skeleton interacting with the active center of the chymotrypsin (table 2). Meanwhile, polypeptide CH59-CH62 (table 1) substituted by amino acid residues at sites P4 and P7′ is synthesized, the inhibitory activity is slightly weaker than that of CH57, IC₅₀ value for inhibiting chymotrypsin is 0.18 μM, 0.09 UM, 0.12 μM and 0.15 μM (table 2, FIG. 6), respectively. A series of disulfide bond expanded polypeptides CH65-CH71 are also synthesized on the basis of CH57 (table 1), wherein CH65 and CH68-CH71 all show better chymotrypsin inhibitory activity, and their IC₅₀ value thereof is 0.48 μM, 0.45 μM, 0.30 μM, 0.25 μM and 0.41 μM (table 2, and FIG. 7), respectively.

TABLE 1
The structure and activities of peptides against chymotrypsin

| | | | | | Theoretical |
|---|---|---|---|---|---|
| SEQ | | | IC₅₀ | Kᵢ | molecular |
| ID NO. | Name | Amino acdi sequences^{a} | (μM) | (μM) | weight (Da) |
| 1 | CH54 | GRCTFSIPPICFPD | 0.03 | 0.01 | 1550.83 |
| 2 | CH55 | GRCTYSIPPICFPD | 0.02 | 0.0086 | 1566.83 |
| 3 | CH56 | GRCTYSIPPICFP | 0.62 | 0.27 | 1451.74 |
| 4 | CH57 | GRCTYSIPPICF | 0.04 | 0.02 | 1354.62 |
| 5 | CH58 | RCTYSIPPICF | 0.27 | 0.12 | 1297.57 |
| 6 | CH59 | GTCTFSIPPICNPN | 0.18 | 0.08 | 1461.69 |
| 7 | CH60 | GTCTYSIPPICNPN | 0.09 | 0.04 | 1477.69 |
| 8 | CH61 | GTCTYSIPPICNP | 0.12 | 0.05 | 1363.58 |
| 9 | CH62 | GTCTYSIPPICN | 0.15 | 0.06 | 1266.47 |
| 10 | CH63 | TCTYSIPPICN | 3.05 | 1.32 | 1209.41 |
| 11 | CH64 | KCTYSIPPICF | 0.93 | 0.40 | 1269.56 |
| 12 | CH65 | CGRATYSIPPICFPD | 0.48 | 0.21 | 1637.90 |
| 13 | CH66 | CGRATYSIPPICFP | 0.65 | 0.28 | 1522.82 |
| 14 | CH67 | CGRATYSIPPICF | 2.00 | 0.86 | 1425.70 |
| 15 | CH68 | CGRXaaTYSIPPICF | 0.45 | 0.19 | 1439.74 |
| 16 | CH69 | CKRATYSIPPICF | 0.30 | 0.13 | 1496.82 |
| 17 | CH70 | CERATYSIPPICF | 0.25 | 0.11 | 1497.76 |
| 18 | CH71 | CGRATFSIPPICF | 0.41 | 0.18 | 1409.70 |
| 19 | CH10 | FCTYSIPPQCYG | 0.06 | 0.03 | 1376.58 |
| 20 | CH5 | FCTFSIPPQCYG | 0.19 | 0.08 | 1360.58 |
| 21 | CH11 | SCTFSIPPQCYG | 0.35 | 0.15 | 1300.48 |
| 22 | CH18 | ACTYSIPAKCF | 0.47 | 0.20 | 1201.44 |
| 23 | CH25 | GTCTFSIPPICNPN | 0.28 | 0.12 | 1461.69 |
| 24 | CH26 | GTCTFSIPPICN | 0.54 | 0.23 | 1250.47 |
| 25 | CH33 | FCTYSIPPQCY | 0.44 | 0.19 | 1319.53 |
| 26 | CH34 | FCTYSIPPQCYA | 0.37 | 0.16 | 1390.61 |
| 27 | CH35 | FCTYSIPPQCR | 0.45 | 0.19 | 1312.54 |
| 28 | CH51 | PCTYSIPPQCY | 0.46 | 0.20 | 1269.47 |

a, A disulfide bond is formed between two cysteine residues within the peptide scaffold against chymotrypsin.; Xaa=L-α-Aminobutyric acid, Abu.

TABLE 2
Determination of the activities of peptides against chymotrypsin

| | Residual | | Residual | | Residual | | Residual |
|---|---|---|---|---|---|---|---|
| | activity of | | activity of | | activity of | | activity of |
| CH10 | chymotrypsin | CH54 | chymotrypsin | CH55 | chymotrypsin | CH57 | chymotrypsin |
| (μM) | (%) | (μM) | (%) | (μM) | (%) | (μM) | (%) |
| 0.0001 | 101.3* | 0.0001 | 100.6 ± 0.9 | 0.0001 | 102.1 ± 1.0 | 0.0001 | 99.0 ± 1.3 |
| 0.01 | 95.1 ± 1.2 | 0.001 | 101.7 ± 0.6 | 0.001 | 101.6 ± 0.9 | 0.001 | 100.2 ± 0.3 |
| 0.1 | 35.4 ± 7.2 | 0.01 | 100.1 ± 2.9 | 0.015 | 68.1 ± 3.7 | 0.01 | 98.0 ± 5.1 |
| 0.2 | 24.9 ± 3.5 | 0.02 | 68.8 ± 4.0 | 0.02 | 45.8 ± 10.0 | 0.02 | 66.0 ± 7.5 |
| 0.4 | 18 ± 4.9 | 0.04 | 34.2 ± 3.9 | 0.04 | 21.8 ± 4.2 | 0.04 | 48.4 ± 5.8 |
| 1 | 3.8 ± 0.4 | 0.08 | 15.2 ± 1.9 | 0.08 | 11.6 ± 1.2 | 0.06 | 38.1 ± 7.9 |
| 10 | 0.1 ± 0.1 | 0.1 | 13.8 ± 3.3 | 0.1 | 8.0 ± 2.4 | 0.08 | 19.1 ± 4.6 |
| 100 | −0.1 ± 0.0 | 1 | 0.4 ± 0.2 | 1 | 0.9 ± 0.1 | 0.1 | 10.3 ± 1.7 |
| | | 10 | −0.1 ± 0.0 | 10 | 0.1 ± 0.0 | 1 | 0.9 ± 0.3 |
| | | | | | | 10 | −0.2 ± 0.2 |

*the concentration of CH10 is almost free of enzyme inhibition activity at the concentration of 0.0001 μM, but the values of the two experimental replicates are abandoned due to the fact that the sample adding error of the two experimental replicates is large.

TABLE 2
Determination of the activities of peptides against chymotrypsin

| | Residual | | Residual | | Residual | | Residual |
|---|---|---|---|---|---|---|---|
| | activity of | | activity of | | activity of | | activity of |
| CH10 | chymotrypsin | CH60 | chymotrypsin | CH61 | chymotrypsin | CH62 | chymotrypsin |
| (μM) | (%) | (μM) | (%) | (μM) | (%) | (μM) | (%) |
| 0.0001 | 101.3* | 0.0001 | 104.2 ± 0.8 | 0.0001 | 100.9 ± 0.6 | 0.0001 | 100.9 ± 0.8 |
| 0.01 | 95.1 ± 1.2 | 0.001 | 104.5 ± 0.9 | 0.001 | 100.1 ± 0.7 | 0.01 | 101.5 ± 2.2 |
| 0.1 | 35.4 ± 7.2 | 0.01 | 101.5 ± 7.6 | 0.01 | 97.3 ± 5.4 | 0.05 | 83.4 ± 8.3 |
| 0.2 | 24.9 ± 3.5 | 0.1 | 47.6 ± 7.6 | 0.05 | 86.1 ± 3.3 | 0.1 | 56.4 ± 2.0 |
| 0.4 | 18.0 ± 4.9 | 0.25 | 29.5 ± 4.6 | 0.1 | 44.2 ± 4.5 | 0.25 | 40.9 ± 6.8 |
| 1 | 3.8 ± 0.4 | 0.5 | 7.4 ± 1.0 | 0.5 | 24.3 ± 2.5 | 0.5 | 25.1 ± 1.5 |
| 10 | 0.1 ± 0.1 | 1 | 3.0 ± 0.2 | 1 | 13.3 ± 1.7 | 1 | 14.2 ± 5.9 |
| 100 | −0.1 ± 0.0 | 10 | 0.7 ± 0.1 | 10 | 1.7 ± 0.0 | 10 | 0.8 ± 0.1 |
| | | 100 | 0.0 ± 0.2 | 100 | 0.2 ± 0.0 | 100 | 0.1 ± 0.0 |

| | Residual | | Residual | | Residual | | Residual |
|---|---|---|---|---|---|---|---|
| | activity of | | activity of | | activity of | | activity of |
| CH65 | chymotrypsin | CH69 | chymotrypsin | CH70 | chymotrypsin | CH71 | chymotrypsin |
| (μM) | (%) | (μM) | (%) | (μM) | (%) | (μM) | (%) |
| 0.001 | 99.8 ± 2.0 | 0.001 | 101.7 ± 0.3 | 0.001 | 100.2 ± 7.6 | 0.001 | 101.4 ± 0.6 |
| 0.1 | 97.8 ± 4.4 | 0.01 | 101.0 ± 0.2 | 0.01 | 106.2 ± 1.8 | 0.01 | 100.6 ± 1.0 |
| 0.25 | 89.5 ± 2.9 | 0.1 | 100.4 ± 2.5 | 0.1 | 105.8 ± 0.9 | 0.1 | 100.3 ± 0.5 |
| 0.5 | 48.2 ± 3.6 | 0.2 | 78.0 ± 4.0 | 0.15 | 96.5 ± 8.5 | 0.2 | 81.7 ± 3.3 |
| 0.75 | 11.7 ± 1.4 | 0.3 | 51.5 ± 5.8 | 0.2 | 55.0 ± 7.2 | 0.4 | 49.9 ± 6.6 |
| 1 | 8.9 ± 1.4 | 0.5 | 20.1 ± 6.1 | 0.5 | 26.0 ± 2.5 | 0.8 | 23.6 ± 1.5 |
| 10 | 4.1 ± 2.3 | 1 | 12.1 ± 1.1 | 1 | 7.7 ± 0.2 | 1 | 20.7 ± 1.3 |
| 100 | 0.1 ± 0.1 | 10 | 1.3 ± 0.6 | 10 | 0.7 ± 0.3 | 10 | 0.8 ± 0.2 |
| 1000 | 0.2 ± 0.0 | 100 | 0.0 ± 0.0 | 100 | −0.1 ± 0.1 | 100 | 0.1 ± 0.1 |

| | Residual | | Residual | | Residual | | Residual |
|---|---|---|---|---|---|---|---|
| | activity of | | activity of | | activity of | | activity of |
| CH5 | chymotrypsin | CH11 | chymotrypsin | CH18 | chymotrypsin | CH25 | chymotrypsin |
| (μM) | (%) | (μM) | (%) | (μM) | (%) | (μM) | (%) |
| 0.0001 | 96.7 ± 9.3 | 0.001 | 98.5 ± 3.1 | 0.001 | 103.6 ± 0.2 | 0.0001 | 99.7 ± 1.0 |
| 0.001 | 113.2 ± 2.4 | 0.01 | 104.1 ± 1.8 | 0.01 | 102.2 ± 3.7 | 0.001 | 100.1 ± 1.9 |
| 0.01 | 102.5 ± 3.7 | 0.1 | 95.3 ± 3.0 | 0.1 | 102.4 ± 2.4 | 0.01 | 96.9 ± 1.4 |
| 0.15 | 58.3 ± 2.3 | 0.15 | 82.3 ± 3.3 | 0.2 | 90.3 ± 4.1 | 0.2 | 63.1 ± 4.1 |
| 0.3 | 41.9 ± 5.7 | 1 | 17.6 ± 0.8 | 0.5 | 49.4 ± 3.7 | 0.3 | 44.6 ± 4.9 |
| 0.6 | 19.0 ± 0.8 | 5 | 12.5 ± 0.7 | 2.5 | 3.7 ± 0.5 | 1 | 15.4 ± 1.2 |
| 1 | 11.3 ± 1.5 | 10 | 6.5 ± 0.3 | 10 | 3.4 ± 0.5 | 10 | 1.5 ± 0.3 |
| 10 | 0.3 ± 0.0 | 100 | 1.1 ± 0.2 | 100 | 0.3 ± 0.1 | 100 | 0.2 ± 0.2 |
| 100 | 0.3 ± 0.2 | 300 | 0.2 ± 0.6 | 1000 | 0.1 ± 0.1 | 1000 | 0.2 ± 0.1 |

| | Residual | | Residual | | Residual |
|---|---|---|---|---|---|
| | activity of | | activity of | | activity of |
| CH33 | chymotrypsin | CH34 | chymotrypsin | CH35 | chymotrypsin |
| (μM) | (%) | (μM) | (%) | (μM) | (%) |
| 0.001 | 99.5 ± 1.4 | 0.001 | 102.3 ± 0.8 | 0.001 | 100.8 ± 0.9 |
| 0.01 | 92.1 ± 2.1 | 0.01 | 104.1 ± 0.5 | 0.01 | 100.7 ± 1.2 |
| 0.1 | 93.6 ± 1.6 | 0.1 | 104.8 ± 0.6 | 0.1 | 100.8 ± 2.6 |
| 0.5 | 42.7 ± 3.1 | 0.2 | 78.5 ± 2.5 | 0.2 | 78.9 ± 2.9 |
| 0.75 | 24.6 ± 2.0 | 0.4 | 45.5 ± 1.5 | 0.4 | 58.8 ± 3.4 |
| 1 | 19.8 ± 1.0 | 0.75 | 24.5 ± 0.9 | 0.8 | 27.3 ± 2.9 |
| 10 | 2.0 ± 0.2 | 1 | 17.8 ± 0.9 | 1 | 16.6 ± 4.7 |
| 100 | 0.1 ± 0.0 | 10 | 1.2 ± 0.1 | 10 | 2.6 ± 0.2 |
| 1000 | 0.1 ± 0.0 | 100 | 0.1 ± 0.0 | 100 | 0.0 ± 0.1 |

| | Residual | | Residual |
|---|---|---|---|
| | activity of | | activity of |
| CH26 | chymotrypsin | CH51 | chymotrypsin |
| (μM) | (%) | (μM) | (%) |
| 0.001 | 101.1 ± 1.6 | 0.001 | 100.2 ± 1.2 |
| 0.1 | 95.1 ± 2.1 | 0.01 | 100.0 ± 0.2 |
| 0.2 | 83.1 ± 1.1 | 0.1 | 95.4 ± 2.2 |
| 0.4 | 63.2 ± 4.5 | 0.2 | 80.3 ± 3.9 |
| 1 | 27.8 ± 2.5 | 0.4 | 54.2 ± 1.8 |
| 5 | 7.0 ± 0.5 | 0.8 | 31.2 ± 1.6 |
| 10 | 2.2 ± 0.2 | 1 | 24.3 ± 2.4 |
| 100 | 0.3 ± 0.1 | 10 | 3.0 ± 0.2 |
| 1000 | 0.1 ± 0.0 | 100 | 0.3 ± 0.0 |

*the concentration of CH10 is almost free of enzyme inhibition activity at the concentration of 0.0001 μM, but the values of the two experimental replicates are abandoned due to the fact that the sample adding error of the two experimental replicates is large.

### Example 3. Construction of methanol yeast expression vector pDeut-Aox1-ScKex2

When the secretory expression of heterologous protein is carried out by using Pichia yeast strain as a host, the most used extensive vector is pPIC9K expression plasmid (Invitrogen). The expression vector has the problems such as unstable expression, only one multiple cloning site (MCS), and low homologous integration efficiency. In order to improve the integration and expression of heterologous target genes, the expression plasmid was modified, and the construction process was as follows:

(1) A promoter and a terminator of cytochrome C isoform 1 (CYC1) of Saccharomyces cerevisiae are artificially synthesized, and then fused with Escherichia coli KAN coding gene (SEQ ID NO.90). The fusion gene fragment amplified by using a pair of primers KAN-1/KAN-2 (SEQ ID NO: 91 and 92) was directly subcloned into the original pPIC9K plasmid expression vector by using restriction endonuclease BsrG I and Nde I. After the ligation (ClonExpress® II One Step Cloning Kit), transformation, screening for positive clones and sequencing validation, the vector pScCycl-Kan was obtained.

(2) A gene fragment (SEQ ID NO: 93) containing alpha 1 mating factor (αMF) signal peptide and secretion preregion of S. cerevisiae αMF, a FLD1 terminator, and a DAS2 promoter was artificially synthesized. Then the fusion gene was amplified using primers A4-B-1/A4-B-2 (SEQ ID NO: 94 and 95), and subcloned into pScCYC-Kan plasmid using restriction endonucleases BamH I. After the ligation (ClonExpress® II One Step Cloning Kit), transformation, screening for positive clones and sequencing validation, the vector pA4-DAS2-Kan was obtained.

(3) The homologous DNA fragment at the 3′-end of the AOX1 gene (SEQ ID NO: 98) was amplified using the genome DNA of Pichia yeast as a template and primers AOX1-3F1/AOX1-3F2 (SEQ ID NO: 96 and 97), and then replaced the AOX1 terminator region of plasmid pA4-DAS2-Kan using the restriction enzyme Sap I. After the ligation (ClonExpress® II One Step Cloning Kit), transformation, screening for positive clones and sequencing validation, the vector pDeut-Aox1 was obtained.

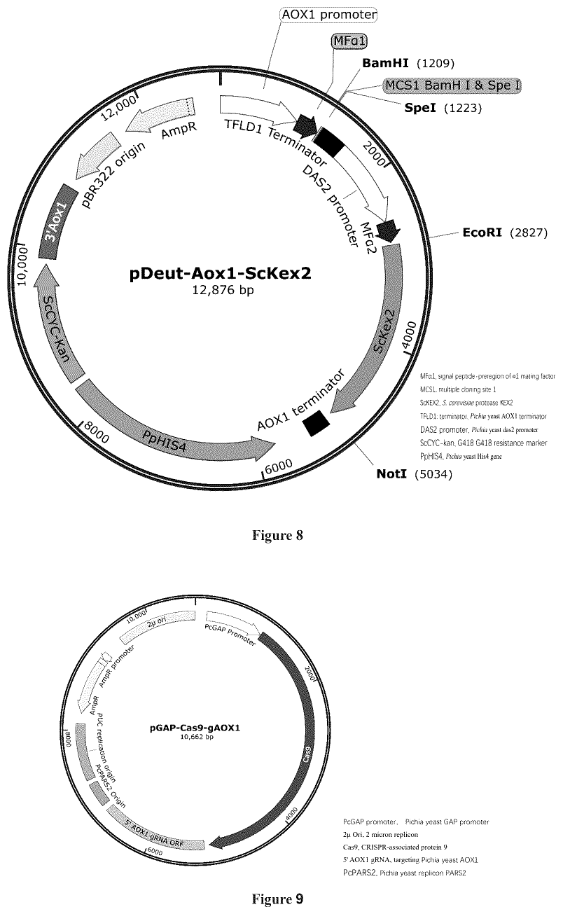

(1) In order to promote the secretion expression of precursor protein of insulin or analog thereof, the expression of protease KEX2 needs to be enhanced. The pair of primer SCKEX2-E1/SCKEX2-N2 (SEQ ID NO: 99 and 100) is used directly to amplify S. cerevisiae protease KEX2 gene with the optimized coding frame (ScKex2, SEQ ID NO: 101) as a template, and then subcloned into the vector pDeut-Aox1 using restriction endonuclease EcoR I and Not I to realize the expression of protease KEX2 under the control of the DDAS2 promoter. A plasmid expression vector pDeut-Aox1-SCKEx2 (FIG. 8) was obtained by means of ligation (ClonExpress® II One Step Cloning Kit), transformation, screening for positive clones and sequencing validation.

### Example 4. Construction of Gene Editing Expression Vector pGAD-Cas9-gAOX1 for Targeting AOX1 Loci in Pichia Yeast

The efficiency of homologous integration of Pichia yeast is very low. In order to improve the site-direct integration efficiency of genes into the AOX1 loci in the yeast, the present invention uses gene editing technology to perform site-direct integration and express the target gene through a gRNA-mediated double-plasmid transformation system. The plasmid pGADT7 is used as the scaffold to construct the gRNA expression vector targeting the AOX1 gene loci. The construction process is as follows:

(1) First, a gRNA expression cassette (gAOX1) targeting the AOX1 promoter region and a Pichcia yeast replicon element PARS2 (SEQ ID NO: 102) were artificially synthesized. Then the primers gRNA-Nhe1/PARS2-Not1 (SEQ ID NO: 103 and 104) were used to amplify the fusion gene, which was subcloned into plasmid pGADT7 via using restriction endonucleases Nhe I and Not I. After the ligation (ClonExpress® II One Step Cloning Kit), transformation, screening for positive clones and sequencing validation, the vector pGADT7-gAOX1 was obtained.

(2) The plasmid pX260 (AddGene) containing a Cas9 coding frame is used as a template, and a pair of primers X260-NDE 1/X260-XHO 1 (SEQ ID NO. 105 and 106) is used directly to amplify the Cas9 encoding frame, and then inserted into the plasmid pGADT7-gAOX1 using restriction endonucleases Nde I and Xho I. After the ligation (ClonExpress® II One Step Cloning Kit), transformation, screening for positive clones and sequencing validation, the vector pCas9-gAOX1 was obtained.

(3) A pair of primers GAP_X260-1/GAP_X260-2 (SEQ ID NO. 107 and 108) and the genome DNA of Pichia yeast as the template were used to amplify the DNA fragment of triphosphoglyceraldehyde dehydrogenase (GAD) gene promoter (SEQ ID NO: 109), and then subcloned into the plasmid pCas9-gAOX1 using a restriction endonuclease Cla I and Nde I site. After the ligation (ClonExpress® II One Step Cloning Kit), transformation, screening for positive clones and sequencing validation, the vector pGAD-Cas9-gAOX1 was obtained (FIG. 9).

### Example 5. Construction of Precursor Protein Expression Vector Containing Hybrid Peptides with Insulin or Analogs

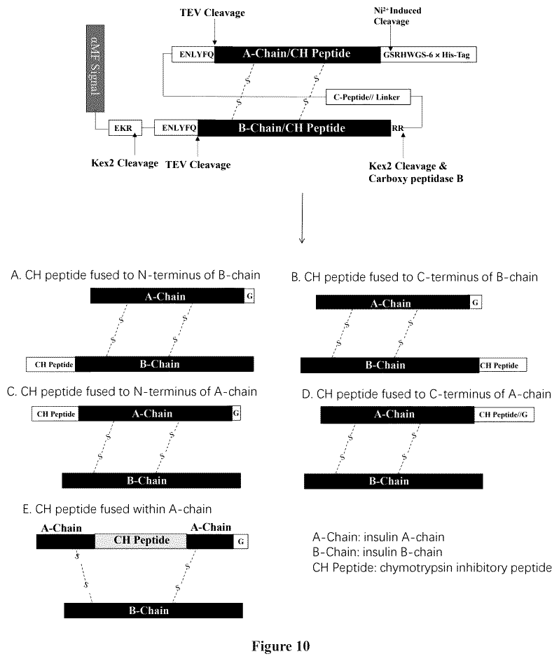

The Pichia yeast cells are eukaryotic mononuclear cells with strong secretory expression capacity and are easy to ferment and culture. The secretory expression of the precursor protein of hybrid peptides is guided by using the signal peptide and the preregion of the S. cerevisiae αMF. Firstly, the precursor protein of the hybrid peptides produced by endoplasmic reticulum (ER) translation is cleaved by a signal peptidase to remove the signal peptide of αMF, and then transported to the trans-Golgi apparatus. The precursor region of αMF and the C-peptide or linker peptide between the B-chain and A-chain in precursor protein of the insulin hybrid peptides or its analogs are cleaved by yeast protease ScKex2, which highly expresses and locates the trans-Golgi apparatus, and then packaged and secreted into the extracellular space. After purification by affinity chromatography, the fused peptide segments and its linker peptides were removed using tobacco etch virus (TEV) protease cleavage, and finally purified to obtain the target protein. FIG. 10 briefly summarizes the process from the precursor protein of the hybrid peptides to the mature hybrid peptide. The detailed construction process of the coding frame for the precursor protein of hybrid peptides is as follows:

### (1) Expression Vector for Precursor Proteins of Human Insulin or Analogs:

The genes encoding precursor proteins of insulin or its analogues with different B-chains and the same A-chain (SEQ ID NO: 29) were artificially synthesized (Table 3). The DNA coding frame were directly amplified using the synthesized primers Ins-BamH1/Ins-Spel (SEQ ID NO: 118 and 119) (Table 4), and then subcloned into the expression plasmid pDeut-Aox1-ScKex2 using restriction endonucleases BamH I and Spe I. After the ligation (ClonExpress® II One Step Cloning Kit), transformation, screening for positive clones and sequencing validation, the expression plasmid vectors pAOX1-ScKex2-Ins-1, pAOX1-ScKex2-Ins-2, pAOX1-ScKex2-Ins-3, and pAOX1-ScKex2-Ins-4 were obtained. The expression plasmids of the correct positive clones were purified, linearized using restriction enzyme Pme I, and then electro-transformed into Pichia pastoris GS115 together with gRNA expression plasmid pGAD-Cas9-gAOX1.

TABLE 3
Genes encoding precursor proteins of human insulin or its analogues

| | Amino acid sequence | |
|---|---|---|
| B- | of precursor protein | Gene sequene encoding precursor protein |
| chain | containing C-peptide | containing C-peptide (5′→3′) |
| SEQ | ENLYFQFVNQHLCG | GAGAATCTGTACTTCCAATTTGTGAACCAACACCTGTG |
| ID | SHLVEALYLVCGER | CGGCTCACACCTGGTGGAAGCTCTGTACCTGGTGTGCG |
| NO: | GFFYTPKTRREAED | GAGAACGTGGATTCTTCTACACACCAAAGACCAGACGT |
| 34 | LQVGQVELGGGPG | GAGGCAGAGGACTTGCAGGTGGGACAGGTGGAGCTGG |
| | AGSLQPLALGENLY | GAGGTGGTCCTGGTGCAGGCAGCCTGCAGCCATTGGCA |
| | FQGIVEQCCTSICSL | CTGGGGGAGAATCTGTACTTCCAAGGCATTGTGGAACA |
| | YQLENYCNGSRHW | ATGCTGTACCAGCATCTGCTCCCTCTACCAGCTGGAGA |
| | GSHHHHHHH | ACTACTGCAACGGATCCAGACATTGGGGATCACATCAT |
| | (SEQ ID NO: 110) | CACCATCATCATCACTAGT (SEQ ID NO: 111) |
| SEQ | ENLYFQFVNQHLCG | GAGAATCTGTACTTCCAATTTGTGAACCAACACCTGTG |
| ID | SHLVEALYLVCGER | CGGCTCACACCTGGTGGAAGCTCTGTACCTGGTGTGCG |
| NO: | GFFYTDKTRREAED | GAGAACGTGGATTCTTCTACACAGATAAGACCAGACGT |
| 35 | LQVGQVELGGGPG | GAGGCAGAGGACTTGCAGGTGGGACAGGTGGAGCTGG |
| | AGSLQPLALGENLY | GAGGTGGTCCTGGTGCAGGCAGCCTGCAGCCATTGGCA |
| | FQGIVEQCCTSICSL | CTGGGGGAGAATCTGTACTTCCAAGGCATTGTGGAACA |
| | YQLENYCNGSRHW | ATGCTGTACCAGCATCTGCTCCCTCTACCAGCTGGAGA |
| | GSHHHHHHH (SEQ | ACTACTGCAACGGATCCAGACATTGGGGATCACAT |
| | ID NO: 112) | CATCACCATCATCATCACTAGT (SEQ ID NO: 113) |
| SEQ | ENLYFQFVNQHLCG | GAGAATCTGTACTTCCAATTTGTGAACCAACACCTGTG |
| ID | SHLVEALYLVCGER | CGGCTCACACCTGGTGGAAGCTCTGTACCTGGTGTGCG |
| NO: | GFFYTKPTRREAED | GAGAACGTGGATTCTTCTACACAAAGCCAACCAGACGT |
| 36 | LQVGQVELGGGPG | GAGGCAGAGGACTTGCAGGTGGGACAGGTGGAGCTGG |
| | AGSLQPLALGENLY | GAGGTGGTCCTGGTGCAGGCAGCCTGCAGCCATTGGCA |
| | FQGIVEQCCTSICSL | CTGGGGGAGAATCTGTACTTCCAAGGCATTGTGGAACA |
| | YQLENYCNGSRHW | ATGCTGTACCAGCATCTGCTCCCTCTACCAGCTGGAGA |
| | GSHHHHHHH (SEQ | ACTACTGCAACGGATCCAGACATTGGGGATCACATCAT |
| | ID NO: 114) | CACCATCATCATCACTAGT (SEQ ID NO: 115) |
| SEQ | ENLYFQFVNQHLCG | GAGAATCTGTACTTCCAATTTGTGAACCAACACCTGTG |
| ID | SHLVEALYLVCGEE | CGGCTCACACCTGGTGGAAGCTCTGTACCTGGTGTGCG |
| NO: | GFFYTKPTRREAED | GAGAAGAGGGATTCTTCTACACAAAGCCAACCAGACG |
| 39 | LQVGQVELGGGPG | TGAGGCAGAGGACTTGCAGGTGGGACAGGTGGAGCTG |
| | AGSLQPLALGENLY | GGAGGTGGTCCTGGTGCAGGCAGCCTGCAGCCATTGGC |
| | FQGIVEQCCTSICSL | ACTGGGGGAGAATCTGTACTTCCAAGGCATTGTGGAAC |
| | YQLENYCNGSRHW | AATGCTGTACCAGCATCTGCTCCCTCTACCAGCTGGAG |
| | GSHHHHHHH (SEQ | AACTACTGCAACGGATCCAGACATTGGGGATCACATCA |
| | ID NO: 116) | TCACCATCATCATCACTAGT (SEQ ID NO: 117) |

### (2) Precursor Protein Expression Vector of Human Insulin or Analog Containing the Fusion of Chymotrypsin Inhibitory Peptide to N-Terminus of B-Chain

Using the constructed expression plasmids pAOX1-ScKex2-Ins-1, pAOX1-ScKex2-Ins-2, pAOX1-ScKex2-Ins-3, and pAOX1-ScKex2-Ins-4 containing the ORFs of precursor protein of human insulin or its analog as templates, the DNA fragments encoding the signal peptide and preregion of αMF were amplified using primers AOX1-P1/CH57-B-N1 or AOX1-P1/CH10-B-N1 (Table 4), respectively. Similarly, the DNA fragments encoding precursor proteins of insulin or analog were amplified using primers CH57-B-N2/TFLD1-R1 or CH10-B-N2/TFLD1-R1 (Table 4). The two DNA fragments amplified were subjected to overlap extension polymerase chain reaction (SOE-PCR), and the resulting SOE-PCR products as the templates were used to amplify the fusion ORFs encoding human insulin or analog with the fusion of chymotrypsin inhibitory peptide to N-terminus of B-chain using the primers Ins-BamH1-2/Ins-Spel (or Ins-Spel-1), and the resulting DNA fragments were subcloned into the expression vector pDeut-Aox1-ScKex2. After the ligation (ClonExpress® II One Step Cloning Kit), transformation, screening for positive clones and sequencing validation, the expression plasmid vectors pIns1-B-CH57N, pIns2-B-CH57N, pIns3-B-CH57N, pIns4-B-CH57N, pIns1-B-CH10N, pIns2-B-CH10N, pIns3-B-CH10N, and pIns4-B-CH10N were obtained.

### (3) Precursor Protein Expression Vector of Human Insulin or Analog Containing the Fusion of Chymotrypsin Inhibitory Peptide to C-Terminus of B-Chain:

Using the constructed expression plasmids pAOX1-ScKex2-Ins-1, pAOX1-ScKex2-Ins-2, pAOX1-ScKex2-Ins-3, and pAOX1-ScKex2-Ins-4 containing the ORFs of precursor protein of human insulin or its analog as templates, the DNA fragments encoding the signal peptide and preregion of αMF along with the B-chain of human insulin or analog were amplified using the pairs of primers AOX1-P1/Ins1-B-CH57C1 or AOX1-P1/Ins1-B-CH10C1, AOX1-P1/Ins2-B-CH57C1 or AOX1-P1/Ins2-B-CH10C1, AOX1-P1/Ins3-B-CH57C1 or AOX1-P1/Ins3-B-CH10C1 and AOX1-P1/Ins3-B-CH57C1 or AOX1-P1/Ins3-B-CH10C1 (Table 4), respectively. Similarly, the DNA fragments encoding the linker peptide and A-chain of insulin or analog were amplified using primers Ins1-B-CH57C2/TFLD1-R1 or Ins1-B-CH10C2/TFLD1-R1 (Table 4). The two DNA fragments amplified were subjected to overlap extension polymerase chain reaction (SOE-PCR), and the resulting SOE-PCR products as the templates were used to amplify the fusion ORFs encoding human insulin or analog with the fusion of chymotrypsin inhibitory peptide to C-terminus of B-chain using the primers Ins-BamH1-2/Ins-Spel (or Ins-Spel-1), and the resulting DNA fragments were subcloned into the expression vector pDeut-Aox1-ScKex2. After the ligation (ClonExpress® II One Step Cloning Kit), transformation, screening for positive clones and sequencing validation, the expression plasmid vectors pIns1-B-CH57C, pIns2-B-CH57C, pIns3-B-CH57C, pIns4-B-CH57C, pIns1-B-CH10C, pIns2-B-CH10C, pIns3-B-CH10C and pIns4-B-CH10C were obtained.

Using the constructed expression plasmids pIns1-B-CH57C, pIns2-B-CH57C, pIns3-B-CH57C, pIns4-B-CH57C, pIns1-B-CH10C, pIns2-B-CH10C, pIns3-B-CH10C and pIns4-B-CH10C as templates, the DNA fragments encoding αMF and human insulin or analog with the fusion of chymotrypsin inhibitory peptide to C-terminus of B-chain were amplified using the pairs of primers AOX1-P1/CH57-G-R1 or AOX1-P1/CH10-G-R1 (Table 4). Similarly, the DNA fragments encoding the linker peptide and A-chain of insulin or analog were amplified using primers CH-G-F1/TFLD1-R1 (Table 4). The two DNA fragments (eight combining forms) amplified were subjected to overlap extension polymerase chain reaction (SOE-PCR), and the resulting SOE-PCR products as the templates were used to amplify the fusion ORFs encoding human insulin or analog with the fusion of chymotrypsin inhibitory peptide to C-terminus of B-chain and C-peptide replaced with a flexible linker (GGSGGSG) using the primers Ins-BamH1-2/Ins-Spel, and the resulting DNA fragments were subcloned into the expression vector pDeut-Aox1-ScKex2. After the ligation (ClonExpress® II One Step Cloning Kit), transformation, screening for positive clones and sequencing validation, the expression plasmid vectors pIns1-B-CH57CT, pIns2-B-CH57CT, pIns3-B-CH57CT, pIns4-B-CH57CT, pIns1-B-CH1OCT, pIns2-B-CH1OCT, pIns3-B-CH1OCT and pIns4-B-CH1OCT were obtained.

### (4) Precursor Protein Expression Vector of Human Insulin or Analog Containing the Fusion of Chymotrypsin Inhibitory Peptide to N-Terminus of A-Chain:

Using the constructed expression plasmids pAOX1-ScKex2-Ins-1, pAOX1-ScKex2-Ins-2, pAOX1-ScKex2-Ins-3, and pAOX1-ScKex2-Ins-4 containing the ORFs of precursor protein of human insulin or its analog as templates, the DNA fragments encoding the signal peptide and preregion of αMF along with the B-chain and the linker peptide of human insulin or analog were amplified using the pairs of primers AOX1-P1/Ins-A-CH57N1 and AOX1-P1/Ins-A-CH10N1 (Table 4), respectively. Similarly, the DNA fragments encoding the linker peptide and A-chain of insulin or analog were amplified using primers Ins-A-CH57N2/TFLD1-R1 or Ins-A-CH10N2/TFLD1-R1 (Table 4). The two DNA fragments amplified were subjected to overlap extension polymerase chain reaction (SOE-PCR), and the resulting SOE-PCR products as the templates were used to amplify the fusion ORFs encoding human insulin or analog with the fusion of chymotrypsin inhibitory peptide to N-terminus of A-chain using the primers Ins-BamH1-2/Ins-Spel (or Ins-Spel-1), and the resulting DNA fragments were subcloned into the expression vector pDeut-Aox1-ScKex2. After the ligation (ClonExpress® II One Step Cloning Kit), transformation, screening for positive clones and sequencing validation, the expression plasmid vectors pIns1-A-CH57N, pIns2-A-CH57N, pIns3-A-CH57N, pIns4-A-CH57N, pIns1-A-CH10N, pIns2-A-CH10N, Ins3-A-CH10N and pIns4-A-CH10N were obtained.

### (5) Precursor Protein Expression Vector of Human Insulin or Analog Containing the Fusion of Chymotrypsin Inhibitory Peptide to C-Terminus of A-Chain:

Using the constructed expression plasmids pAOX1-ScKex2-Ins-1, pAOX1-ScKex2-Ins-2, pAOX1-ScKex2-Ins-3, and pAOX1-ScKex2-Ins-4 containing the ORFs of precursor protein of human insulin or its analog as templates, the DNA fragments encoding the signal peptide and preregion of αMF along with the B-chain, the linker peptide and A-chain of human insulin or analog were amplified using the pairs of primers AOX1-P1/Ins-A-CH57C1 and AOX1-P1/Ins-A-CH10C1 (Table 4), respectively. Similarly, the DNA fragments encoding the linker peptide and A-chain of insulin or analog were amplified using primers Ins-A-CH57C2/TFLD1-R1 or Ins-A-CH10C2/TFLD1-R1 (Table 4). The two DNA fragments amplified were subjected to overlap extension polymerase chain reaction (SOE-PCR), and the resulting SOE-PCR products as the templates were used to amplify the fusion ORFs encoding human insulin or analog with the fusion of chymotrypsin inhibitory peptide to C-terminus of A-chain using the primers Ins-BamH1-2/Ins-Spel, and the resulting DNA fragments were subcloned into the expression vector pDeut-Aox1-ScKex2. After the ligation (ClonExpress® II One Step Cloning Kit), transformation, screening for positive clones and sequencing validation, the expression plasmid vectors pIns1-A-CH57C, pIns2-A-CH57C, pIns3-A-CH57C, pIns4-A-CH57C, pIns1-A-CH10C, pIns2-A-CH10C, pIns3-A-CH10C and pIns4-A-CH10C were obtained.

Using the constructed expression plasmids pIns1-A-CH57C, pIns2-A-CH57C, pIns3-A-CH57C, pIns4-A-CH57C, pIns1-A-CH10C, pIns2-A-CH10C, pIns3-A-CH10C and pIns4-A-CH10C as templates, the DNA fragments encoding αMF and human insulin or analog with the fusion of chymotrypsin inhibitory peptide to C-terminus of B-chain were amplified using the pair of primers AOX1-P1/A-CH-LR1 (Table 4). Similarly, the DNA fragments encoding the linker peptide and A-chain of insulin or analog were amplified using primers A-CH-LR2/TFLD1-R1 (Table 4). The two DNA fragments (eight combining forms) amplified were subjected to overlap extension polymerase chain reaction (SOE-PCR), and the resulting SOE-PCR products as the templates were used to amplify the fusion ORFs encoding human insulin or analog with the fusion of chymotrypsin inhibitory peptide to C-terminus of A-chain and linkr peptide replaced with a flexible linker SEQ ID NO: 70 (RREAEDGSGGSGENLYFQ) using the primers Ins-BamH1-2/Ins-Spel, and the resulting DNA fragments were subcloned into the expression vector pDeut-Aox1-ScKex2. After the ligation (ClonExpress® II One Step Cloning Kit), transformation, screening for positive clones and sequencing validation, the expression plasmid vectors pIns1-A-CH57CT, pIns2-A-CH57CT, pIns3-A-CH57CT, pIns4-A-CH57CT, pIns1-A-CH1OCT, pIns2-A-CH1OCT, pIns3-A-CH10CT and pIns4-A-CH1OCT were obtained.

(6) Precursor Protein Expression Vector of Human Insulin or Analog Containing the Fusion of Chymotrypsin Inhibitory Peptide within A-Chain:

Using the constructed expression plasmids pAOX1-ScKex2-Ins-1, pAOX1-ScKex2-Ins-2, pAOX1-ScKex2-Ins-3, and pAOX1-ScKex2-Ins-4 containing the ORFs of precursor protein of human insulin or its analog as templates, the DNA fragments encoding the signal peptide and preregion of αMF along with the B-chain, the linker peptide and partial A-chain of human insulin or analog were amplified using the pairs of primers AOX1-P1/Ins-A-CH57M1 and AOX1-P1/Ins-A-CH10M1 (Table 4), respectively. Similarly, the DNA fragments encoding the linker peptide and partial A-chain of insulin or analog were amplified using primers Ins-A-CH57M2/TFLD1-R1 or Ins-A-CH10M2/TFLD1-R1 (Table 4). The two DNA fragments amplified were subjected to overlap extension polymerase chain reaction (SOE-PCR), and the resulting SOE-PCR products as the templates were used to amplify the fusion ORFs encoding human insulin or analog with the fusion of chymotrypsin inhibitory peptide within A-chain using the primers Ins-BamH1-2/Ins-Spel (or Ins-Spel-1), and the resulting DNA fragments were subcloned into the expression vector pDeut-Aox1-ScKex2. After the ligation (ClonExpress® II One Step Cloning Kit), transformation, screening for positive clones and sequencing validation, the expression plasmid vectors pIns1-A-CH57M, pIns2-A-CH57M, pIns3-A-CH57M, pIns4-A-CH57M, pIns1-A-CH10M, pIns2-A-CH10M, pIns3-A-CH10M and pIns4-A-CH10M were obtained.

TABLE 4
Primers used for constructing expression vectors of precursor proteins
of human insulin hybrid peptide or analog thereof

| SEQ ID | | |
|---|---|---|
| NO. | Primer name | Primer sequence, 5′→3′ |
| 118 | Ins-BamH1 | TGAAGCCGAGGCAGGATCCGAGAATCTGTACTTCCAA |
| | | TTTGTGAAC |
| 119 | Ins-SpeI | ATTGACTATACACACTAGTGATGATGATGGTGATGATGT |
| | | GATCCCCAATGTCTGGATCC |
| 120 | CH57-B-N1 | GAATAGAATAAGTACACCTACCTTGGAAGTACAGATT |
| | | CTCGGATCCTGCCTCGGCTTCA |
| 121 | CH57-B-N2 | AGGTGTACTTATTCTATTCCTCCAATATGCTTTGGAGG |
| | | TTTTGTGAACCAACACCTGTG |
| 122 | CH10-B-N1 | AGGAATAGAGTAAGTACAAAATTGGAAGTACAGATT |
| | | CTCGGATCCTGCCTCGGCTTCA |
| 123 | CH10-B-N2 | TTTGTACTTACTCTATTCCTCCACAGTGCTATGGAGGT |
| | | TTTGTGAACCAACACCTGTG |
| 124 | AOX1_P1 | CACATTGTATGCTTCCAAGATTCTG |
| 125 | AOX1_P2 | GAAGATCAAAAAACAACTAATTATTCG |
| 126 | TFLD1-R1 | GATACATATATGCTGGAAATACTCCGGC |
| 127 | Ins-BamH1-2 | TGAAGCCGAGGCAGGATCC |
| 128 | Ins1-B-CH57C1 | AAAGCATATTGGAGGAATAGAATAAGTACACCTACC |
| | | GGTCTTTGGTGTGTAGAAGA |
| 129 | Ins1-B-CH57C2 | TTCTATTCCTCCAATATGCTTTGGATCCAGACATTGGG |
| | | GAGGTGGTCCTGGTGCA |
| 130 | Ins1-B-CH10C1 | ATAGCACTGTGGAGGAATAGAGTAAGTACAAAATCC |
| | | GGTCTTTGGTGTGTAGAAGA |
| 131 | Ins1-B-CH10C2 | CTATTCCTCCACAGTGCTATGGATCCAGACATTGGGG |
| | | AGGTGGTCCTGGTGCA |
| 132 | Ins2-B-CH57C1 | AAAGCATATTGGAGGAATAGAATAAGTACACCTACC |
| | | GGTCTTATCTGTGTAGAAGA |
| 133 | Ins2-B-CH10C1 | ATAGCACTGTGGAGGAATAGAGTAAGTACAAAATCC |
| | | GGTCTTATCTGTGTAGAAGA |
| 134 | Ins3-B-CH57C1 | AAAGCATATTGGAGGAATAGAATAAGTACACCTACC |
| | | GGTTGGCTTTGTGTAGAAGA |
| 135 | Ins3-B-CH10C1 | ATAGCACTGTGGAGGAATAGAGTAAGTACAAAATCC |
| | | GGTTGGCTTTGTGTAGAAGA |
| 136 | CH57-G-R1 | TCCCCAATGTCTGGATCCAAAGCATATTGGAGGAATA |
| | | G |
| 137 | CH10-G-R1 | TCCCCAATGTCTGGATCCATAGCACTGTGGAGGAATA |
| | | G |
| 138 | CH-G-F1 | GGATCCAGACATTGGGGAGGTTCTGGAGGTAGTGGT |
| | | GAGAATCTGTACTTCCAAGGCA |
| 139 | Ins-A-CH57N1 | GAGGAATAGAATAAGTACACCTACCTTGGAAGTACA |
| | | GATTCTCCCCCAGTGCCAATGG |
| 140 | Ins-A-CH57N2 | GTGTACTTATTCTATTCCTCCAATATGCTTTGGAGGCA |
| | | TTGTGGAACAATGCTGTACC |
| 141 | Ins-A-CH10N1 | GAGGAATAGAGTAAGTACAAAATCCTTGGAAGTACA |
| | | GATTCTCCCCCAGTGCCAATGG |
| 142 | Ins-A-CH10N2 | TTGTACTTACTCTATTCCTCCACAGTGCTATGGAGGCA |
| | | TTGTGGAACAATGCTGTACC |
| 143 | Ins-A-CH57C1 | TTGGAGGAATAGAATAAGTACACCTACCTCCGTTGCA |
| | | GTAGTTCTCCAGCTG |
| 144 | Ins-A-CH57C2 | TACTTATTCTATTCCTCCAATATGCTTTGGATCCAGACA |
| | | TTGGGGATCACAT |
| 145 | Ins-A-CH10C1 | GTGGAGGAATAGAGTAAGTACAAAATCCTCCGTTGC |
| | | AGTAGTTCTCCAGCTG |
| 146 | Ins-A-CH10C2 | TACTTACTCTATTCCTCCACAGTGCTATGGATCCAGAC |
| | | ATTGGGGATCACAT |
| 147 | A-CH-LR1 | TCACCACTACCTCCAGAACCGTCCTCTGCCTCACGTCT |
| | | GGT |
| 148 | A-CH-LR2 | ACGGTTCTGGAGGTAGTGGTGAGAATCTGTACTTCCA |
| | | AGG |
| 149 | Ins-A-CH57M1 | ATATTGGAGGAATAGAATAAGTACACCTGCATTGTTC |
| | | CACAATGCCTTG |
| 150 | Ins-A-CH57M2 | CTTATTCTATTCCTCCAATATGCTTTGGATCCCTCTAC |
| | | CAGCTGGAGAAC |
| 151 | Ins-A-CH10M1 | ACTGTGGAGGAATAGAGTAAGTACAAAAGCATTGTT |
| | | CCACAATGCCTTG |
| 152 | Ins-A-CH10M2 | CTTACTCTATTCCTCCACAGTGCTATGGATCCCTCTACC |
| | | AGCTGGAGAAC |
| 153 | Ins-SpeI-1 | CCAGCTGGAGAACTACTGCGGATCCAGACATTGGGGA |
| | | TCACATCATCACCATCATCATCACTAGTGTGTATAGT |
| | | CAAT |

### Example 6: DNA Transformation of Pichia Yeast, Screening of Positive Transformants, and Induction Expression of Target Protein

### Electroporation Method:

(1) pick up a single colony on a newly activated Pichia pastoris GS115 agar plate, and culture them in 5-10 mL YPD liquid medium at 30° C. and 220 rpm for 8-10 hours. The culture GS115 cells are transferred into a fresh YPD medium according to the proportion of 1.2-1.5% (V/V) and cultured overnight under the same condition.

(2) when the OD600 value of the yeast culture is 1.3-1.5, collecting each 4-6 ml of yeast culture into a 1.5 ml centrifuge tube, and centrifuging at 8000 rpm for 5 minutes.

(3) Sequentially wash the yeast cells with 1 mL of pre-cooled sterile water twice and with 1 mL of 1M sterile sorbitol solution twice. After each washing, centrifuge at 8000 rpm for 5 minutes.

(4) Every 4-6 mL of the centrifuged yeast cells is resuspended in 80 μL of 1M pre-cooled sterile sorbitol solution, resulting in a treated tube of competent cells.

(5) Mix a tube of GS115 competent cells with 2-3 μg (not exceeding 10 μL) of the target DNA fragment, add it to a pre-cooled electroporation cup, and let it stand on ice for 5 minutes. Wipe off the water droplets on the outer wall of the electroporation cup, select the “Pic” option for Pichia pastoris on the electroporation apparatus table, record the corresponding parameters after electroporation shock, and immediately add 1 mL of 1 M pre-cooled sterile sorbitol YPD solution. Let it stand on ice for 1 hour. Let it stand at 30° C. for 4-6 hours for rejuvenation. After gently mixing with a pipette, apply every 200 μL of rejuvenated yeast cells onto a YPD resistant plate. Incubate in a 30° C. incubator for 2-4 days.

Extraction of yeast genomic DNA: Follow the steps of the yeast genome extraction kit as follows:

(1) Take 1-5 mL of yeast culture (not exceeding 5× 107 yeast cells), centrifuge at 12000 rpm for 1 minute, and try to remove the supernatant as much as possible.

(2) Breaking of yeast cell wall: Add 600 μL of snail enzyme to the yeast cells, mix thoroughly, and treat on a shaker at 220 rpm and 30° C. for 2 hours. Centrifuge at 4000 rpm for 10 minutes, discard the supernatant, and collect the precipitate.

(3) Add 200 μL of buffer GA and resuspend thoroughly.

(4) Add 20 μL Proteinase K solution and mix well.

(5) Add 220 μL of buffer GB, thoroughly invert and mix, let it stand at 70° C. for 10 minutes, the solution strain becomes clear, and briefly centrifuge to remove water droplets from the tube wall.

(6) Add 220 μL anhydrous ethanol, thoroughly invert and mix, and briefly centrifuge to remove water droplets from the tube wall.

(7) Add the solution and flocculent precipitate obtained in the previous step into an adsorption column CB3 (place the adsorption column in a collection tube), centrifuge at 12000 rpm for 30 seconds, discard the waste liquid, and return it to CB3.

(8) Add 500 μL of buffer solution GD to adsorption column CB3, centrifuge at 12000 rpm for 30 seconds, discard the waste liquid, and return it to CB3.

(9) Add 600 μL of rinse solution PW to adsorption column CB3, centrifuge at 12000 rpm for 30 seconds, discard the waste liquid, and return it to CB3. Repeat once

(10) Place the adsorption column CB3 into a collection tube and centrifuge at 12000 rpm for 2 minutes, then let it stand at room temperature for 10 minutes to remove any residual ethanol in the adsorption column.

(11) Place the adsorption column CB3 in a clean centrifuge tube, add 50 μL ddH2O to the center of the adsorption membrane, incubate at 37° C. for 15 minutes, centrifuge at 12000 rpm for 2 minutes, and collect the genomic DNA solution into the centrifuge tube.

### PCR Identification of Positive Clones:

Screen positive clones using YPD solid medium containing 100 μg·mL-1 G418 resistance. Six positive clones of recombinant strains were randomly selected to extract their genomic DNA. Primers AOX1-P1/TFLD1-R1 were used for the first round of PCR amplification, followed by a second round of PCR validation using primers AOX1-P2/Ins-Spel. The amplified bands of the target gene positive clones were around 1 kb, and the DNA fragments were purified and recovered before sequencing validation.

### Rapid Screening of Target Protein Expression:

Select monoclonal antibodies identified by PCR and inoculate them into 4 mL of BMGY medium. Incubate at 28° C. and 240 RPM for approximately 16-20 hours, with an OD600 of 8-10. Transfer it into 4 mL of BMMY at OD600=1, continue to culture at 28° C. and 240 RPM for 72-96 hours, and add 1-1.5% anhydrous methanol every day. Perform dot blot hybridization according to the operation of Bio-Dot Aparatus (Bio rad), and select clones with strong hybridization signals for shake flask fermentation culture. Dot-blot hybridization is carried out according to the operation of Bio-Dot Apparatus (Bio-Rad), and the clone with strong hybridization signal is selected for shake flask fermentation culture.

### Expression and Purification of Target Protein:

Select positive clones with high expression levels of the target protein through dot blot screening, and induce fermentation culture using a 1-L shake flask containing 250 mL BMMY medium. After 72-96 hours of fermentation and cultivation, collect the supernatant by centrifugation. Then, nickel ion affinity chromatography was used to purify extracellular secreted proteins. The target protein is obtained by using imidazole gradient elution.

### Example 7: Expression, Purification of Protease TEV and PAM, and Digestion Reaction

Construct the expression vector of protease TEV: In the present invention, a cold shock-induced expression system Pcold-MBP (Takara Company) is selected, heterologous expression is carried out using Shuffle T7 host bacteria, the solubility of the protein is increased, and correct folding of the protein is promoted. In order to conveniently purify a target protein by using 6 x His Tag, the primers Cold-NHE1/Cold-NDE 1 (SEQ ID NO:: 154 and 155) (Table 5) were used to amplify the DNA fragment encoding the MBP, replacing the corresponding vector structure fragment on the vector with the restriction endonuclease Nhe I and the Nde I, and eliminating the His-Tag label at the N-terminal of the MBP label for promoting soluble expression. The plasmid pCold-MBP-T was obtained. Next, the tobacco etch virus (TEV) protease coding frame (SEQ ID NO: 158) artificially synthesized was directly amplified utilizing the primers TEV-BamH 1/TEV-SAL 1 (SEQ ID NO: 156 and 157) (Table 5), and then subloned into the pCold-MBP-T by using restriction endonuclease BamH I and Sal I. After the ligation (ClonExpress® II One Step Cloning Kit), transformation, screening for positive clones and sequencing validation, the expression plasmid vectors pCold-TEV was obtained, so that the soluble expression of TEV and the self-cleavage of the fusion label MBP can be realized.

### Construct the Expression Vector of Peptidylglycine Alpha-Amidating Monooxygenase (PAM):

PAM contains two catalytic domains of peptidylglycine alpha-hydroxylating monooxygenase (PHM) and peptidylamidoglycolate lyase (PAL). Using the plasmid pReceiver-M02 (GeneCopoeia) containing the PAM gene (GenBank: NM_000919) as a template, PCR amplification was performed using the primers pReceiver-M03F/PAM-1 (SEQ ID NO: 159 and 161) and PAM-2/pReceiver-M03R (SEQ ID NO: 160 and 162) (Table 5) to amplify DNA fragments containing the PHM domain encoding amino acid residues 39-350 and the PAL domain encoding amino acids 494-835, respectively. These two DNA fragments were then subjected to PCR overlapping reaction, and the overlapped product was used as a template to amplify the truncated PAM mutant, in which a linker peptide segment (GSGGSG) was introduced between the two domains, using primers PAM-BamH1/PAM-Sa11 (SEQ ID NO: 163 and 164) (Table 5). The resulting DNA fragment was inserted into plasmid pCold-MBP-T. After the ligation (ClonExpress® II One Step Cloning Kit), transformation, screening for positive clones and sequencing validation, the expression plasmid vector pCold-PAM was obtained, and the N-terminal fusion tag of MBP can be removed by auto-cleavage of protease TEV.

The above two proteases were purified by nickel ion affinity chromatography to obtain, and then an enzymatic digesting reaction system containing insulin or its analog hybrid peptides was established: 50 mM Hepes (pH 7.0), 1 u M CuSO4, 50 μg/ml catalase, 1 mM ascorbate, precursor protein substrate, protease TVE, and PAM were added. The precursor protein was directly cleaved at 30° C. After the reaction was completed, 1/10 volume of 0.1 M EDTA and 50% TFA were added to inactivate the enzymatic cleavage reaction. Finally, ultrafiltration concentration was used to replace the reaction buffer (1.0 mM NiCl2, 0.1 M CHES, 0.1 M acetone oxime, 0.1 M NaCl, pH 8.2) with nickel ion induced the cleavage of fusion tag (G↓SRHW). The reaction was carried out at 22° C. for 16 hours, and then directly purified by HPLC to obtain the hybrid peptide containing insulin or its analogues provided by the present invention.

TABLE 5
Primers for constructing expression vectors of protease TEV and PAM

| SEQ ID NO. | Primer name | Primer sequence, 5′→3′ |
|---|---|---|
| 154 | Cold-NheI | GCGGATAACAATTTGATGTGCTAGC |
| 155 | Cold-NdeI | TACCTTCTTCGATTTTCATATGCCTACCTTCGATCA |
| | | CTTTGTGATTCATGGT |
| 156 | TEV-BamHI | CGGTACCCTCGAGGGATCCGAGAATCTGTACTTCCA |
| | | AGGAGGCGAGAGCCTGTTCAAGG |
| 157 | TEV-SalI | CCTATCTAGACTGCAGGTCGACCTAATGATGATGA |
| | | TGATGATGACCTCCATTCATCAGCTGAGTGGCCT |
| 159 | PReceiver-M03F | GCGGTAGGCGTGTACGGT |
| 160 | PReceiver-M03R | CCGGACACGCTGAACTTGT |
| 161 | PAM-1 | CACCACTACCTCCAGAACCGGGAATTGGAATGTTG |
| | | GCCTC |
| 162 | PAM-2 | CGGTTCTGGAGGTAGTGGTGGAGATTTCCACATGG |
| | | AAGAG |
| 163 | PAM-BamHI | CGGTACCCTCGAGGGATCCGAGAATCTGTACTTCCA |
| | | AGGATCCAATGAATGTCTTGGTA |
| 164 | PAM-SalI | CCTATCTAGACTGCAGGTCGACCTAATGATGATGA |
| | | TGATGATGACCTCCTTCAACAACTGCCTCGGCTT |

### Example 8: Stability Analysis of Insulin Hybrid Peptides or Analogs thereof against Chymotrypsin Hydrolysis

Control test: Take three sterile EP tubes and add 1.5 μL of 1 mM insulin hybrid peptides or analogs, 23.5 μL of reaction buffer (20 mM CaCl₂, pH 7.8, 50 mM Tris-HCl), and 3.75 μL of 10% TFA. Mix by centrifugation at 8000 rpm for 30 seconds.

Enzymatic hydrolysis kinetics of chymotrypsin on insulin molecules, insulin hybrid pepetides, or analogs against chymotrypsin: Take three sterile EP tubes and add 9 μL of 1 mM insulin molecules, insulin hybrid pepetides, or analogs and 138 μL of reaction buffer (20 mM CaCl₂, pH 7.8, 50 mM Tris-HCl). Arrange a certain volume of 0.05 μg/μL chymotrypsin solution in another sterile EP. Incubate the EP tubes containing peptides and enzymes at 37° C. for 5 min, and add 3 μL of chymotrypsin solution to each EP tube containing peptides and mix well. Start timing and extract 25 μL of reaction solution at 0.5, 1.0, 2.0, 3.0 and 4.0 h, respectively. Add 3.75 μL of 10% TFA to terminate the reaction and mix well by centrifugation at 8000 rpm for 30 seconds.

In the 25 μL of reaction system of two different sets described above, the final concentrations of insulin molecules, insulin hybrid pepetides, or analogs and chymotrypsin were 60 μM and 1.0 ng/μL, respectively. There are three replicates at each time point, and the peak area of the peptide at each time point is detected using reverse phase high-performance liquid chromatography (RP-HPLC). The ratio of the remaining peak area of the sample at detection time T (h) to the peak area of the prototype peptide at 0 h is calculated as the remaining percentage (%) of the peptide.

Results: After 4 hours of chymotrypsin hydrolysis, only about 10.3% of human insulin remained, indicating low stability to chymotrypsin hydrolysis. However, the introduction of insulin analogs containing chymotrypsin inhibitory skeletons CH57 or CH10 showed excellent stability against trypsin hydrolysis, with a remaining amount exceeding nearly 80% after 4 hours of enzymatic hydrolysis.

TABLE 6
Stability analysis of human insulin and insulin hybrid peptides
(Ins3-A-CH10M and Ins3-A-CH57M) against chymotrypsin

| | |
|---|---|
| Time | Remaining peak area (%)/chymotrypsin |

| (h) | Insulin | Ins3-A-CH10M | Ins3-A-CH57M |
|---|---|---|---|
| 0.0 | 100.0 ± 0.0 | 100.0 ± 0.0 | 100.0 ± 0.0 |
| 0.5 | 70.7 ± 3.0 | 89.1 ± 5.8 | 79.8 ± 3.7 |
| 1.0 | 57.4 ± 2.3 | 90.5 ± 3.5 | 79.8 ± 0.9 |
| 2.0 | 36.5 ± 1.0 | 90.3 ± 2.4 | 80.2 ± 0.6 |
| 3.0 | 21.7 ± 2.1 | 93.8 ± 5.4 | 82.5 ± 1.2 |
| 4.0 | 10.3 ± 0.7 | 92.4 ± 0.4 | 80.2 ± 0.9 |

### Example 9 In Vivo Activity of Insulin, Insulin Hybrid Peptides or Analogs Injected Subcutaneously and Administered Through the Duodenum

The in vivo hypoglycemic activity of insulin analog and insulin hybrid peptides administered subcutaneously: Normal ICR mice were subcutaneously injected with HumulinR (0.4 U/kg) and insulin hybrid peptidess or corresponding volumes of physiological saline (0.1 mL/10 g B. W., n=6-9). Tail tip blood samples were collected at 40 min and 90 min after administration, and blood glucose levels were measured using glucose oxidase method to calculate blood glucose values at each blood sampling time point. The calculated values are expressed as “mean±standard error”, and p<0.05 is considered statistically significant.

Result: Compared with a positive control, insulin analogue lispro synthesized in the present invention showed good hypoglycemic activity. Insulin hybrid peptides containing chymotrypsin inhibitory peptides (Ins3-B-CH57N, Ins3-B-CH57C, Ins3-A-CH10M, Ins3-A-CH57M) also showed good hypoglycemic activity (Table 7). It indicates that there is no structural impact of chymotrypsin inhibitory peptide on interaction between insulin hybrid peptides and insulin receptors, whether at the N-terminus of B-chain, C-terminus of B-chain, or in the middle of A-chain of insulin hybrid peptides synthesized in this invention. However, the activity of insulin hybrid peptides with the fusion of chymotrypsin inhibitory peptides to N- or C-terminus of A-chain prepared by biological expression is weak (Ins3-A-CH57N and Ins3-A-CH57C), which may be due to the incorrect formation of disulfide bond structures and may be a false negative result.

TABLE 7
Hypoglycemic activity of subcutaneous administration of insulin and analogs

| | Dosage | | Glucose (mg/dL) (mean ± sd) | | |
|---|---|---|---|---|---|
| Group | (mg/kg) | n | 0 min | 40 min | 90 min |
| Nor | — | 9 | 98.6 ± 26.9 | 95.7 ± 10.3 | 99.1 ± 9.6 |
| lispro | 0.9 | 8 | 96.7 ± 24.8 | 70.3 ± 6.2*** | 72.9 ± 15.6** |
| Ins3-A- | 0.5 | 8 | 86.2 ± 18.9 | 90.4 ± 9.7 | 89.7 ± 18.2 |
| CH57N | | | | | |
| Ins3-A- | 1.1 | 8 | 100.4 ± 11.5 | 92.4 ± 13.9 | 106.0 ± 25.2 |
| CH57C | | | | | |
| Ins3-A- | 0.7 | 8 | 93.1 ± 15.5 | 83.5 ± 7.8* | 107.9 ± 13.4 |
| CH10M | | | | | |
| Ins3-A- | 0.1 | 8 | 97.9 ± 21.8 | 85.3 ± 8.3* | 86.9 ± 19.3 |
| CH57M | | | | | |
| HumulinR | 0.4 U/kg | 8 | 97.1 ± 13.8 | 56.5 ± 9.9*** | 60.3 ± 6.8*** |

| | Dosage | | Glucose (mg/dL) (mean ± sd) | | | |
|---|---|---|---|---|---|---|
| Group | (mg/kg) | n | 0 min | 20 min | 40 min | 90 min |
| Nor | — | 9 | 102.6 ± 20.5 | 109.5 ± 12.0 | 107.3 ± 9.2 | 112.6 ± 22.2 |
| Ins3-B-CH57N | 3.0 | 8 | 107.0 ± 24.1 | 105.4 ± 7.4 | 90.1 ± 13.1** | 112.0 ± 15.3 |
| Ins3-B-CH57C | 2.6 | 6 | 96.1 ± 22.5 | 101.1 ± 10.0 | 85.8 ± 12.7** | 97.0 ± 15.4 |

*p < 0.05;
**p < 0.01;
***p < 0.001.

### In Vivo Hypoglycemic Activity of Insulin Hybrid Peptides or Analogs Administered Via Duodenal Administration:

Normal ICR mice were anesthetized by inhalation of ether, and their duodenum was surgically removed and injected with insulin analogs or corresponding volumes of physiological saline (0.1 mL/10 g B. W., n=6-10). The wound was then sutured, and blood samples were collected from the tail tip at 40 and 90 minutes after administration. Blood glucose levels were measured using glucose oxidase method, and blood glucose values were calculated at each blood sampling time point. The calculated values are expressed as “mean±standard error”, and p<0.05 is considered to have a statistical difference.

Result: Compared with the normal control group, only insulin hybrid peptides Ins3-B-CH57C, Ins3-A-CH10M, and Ins3-A-CH57M significantly reduced blood glucose levels in mice after duodenal administration for 40 minutes, with blood glucose levels reduced by 12.1%, 11.9%, and 15.3%, respectively (Table 8). This indicates that chymotrypsin inhibitory peptides have a good protective effect on insulin at the C-terminus of the B-chain and in the middle of the A-chain, improving their in vivo stability and promoting the absorption of insulin hybrid peptides into the bloodstream through the intestinal epithelium.

TABLE 8
Hypoglycemic activity of human insulin and analogs
administered via duodenal administration

| | Dos- | | | | |
|---|---|---|---|---|---|
| | age | | | | |
| | (mg/ | | Glucose (mg/dL) (mean ± sd) | | |
| Group | kg) | n | 0 min | 40 min | 90 min |
| Nor | — | 10 | 92.7 ± 8.1 | 102.5 ± 10.7 | 86.6 ± 14.4 |
| Ins3-B-CH57N | 0.6 | 9 | 89.3 ± 13.9 | 96.9 ± 9.5 | 97.8 ± 9.6 |
| Ins3-B-CH57C | 0.7 | 6 | 85.1 ± 10.7 | 90.1 ± 6.8* | 95.3 ± 13.8 |
| Ins3-A-CH57N | 0.7 | 9 | 88.6 ± 12.6 | 97.2 ± 14.9 | 93.9 ± 9.3 |
| Ins3-A-CH57C | 0.5 | 9 | 83.1 ± 3.8 | 101.1 ± 11.8 | 99.6 ± 13.9 |
| Nor | — | 7 | 91.5 ± 11.9 | 122.3 ± 14.2 | 105.2 ± 12.7 |
| Ins3-A-CH10M | 7.7 | 9 | 91.0 ± 10.6 | 107.7 ± 12.4* | 101.3 ± 14.2 |
| Ins3-A-CH57M | 6.7 | 8 | 87.5 ± 9.8 | 103.6 ± 11.7* | 99.3 ± 11.9 |

*p < 0.05;
** p < 0.01;
*** p < 0.001.

What is claimed is:

1. A hybrid peptide formed by connection of an insulin or an analog thereof to a chymotrypsin inhibitory peptide via a linker peptide, wherein the insulin or the analog thereof comprises a B-chain and an A-chain linked together by two pairs of interchain disulfide bonds, the chymotrypsin inhibitory peptide is connected to the B-chain or A-chain of the insulin or the analog thereof via the linker peptide.

2. The hybrid peptide according to claim 1, characterized in that the chymotrypsin inhibitory peptide is selected from the group consisting of polypeptides with the following amino acid sequences: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 11, SEQ ID NO: 12, SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28.

3. The hybrid peptide according to claim 1, characterized in that the insulin or the analog thereof comprises an A-chain and a B-chain, wherein the A-chain is selected from the group consisting of polypeptides with the following amino acid sequences: SEQ ID NO: 29, SEQ ID NO: 30, SEQ ID NO: 31, SEQ ID NO: 32, and SEQ ID NO: 33, and the B-chain is selected from the group consisting of polypeptides with the following amino acid sequences: SEQ ID NO: 34, SEQ ID NO: 35, SEQ ID NO: 36, SEQ ID NO: 37, SEQ ID NO: 38, SEQ ID NO: 39, and SEQ ID NO: 40.

4. The hybrid peptide according to claim 1, characterized in that the linker peptide between the insulin or the analog thereof and the chymotrypsin inhibitory peptide is a peptide fragment comprising Gly, Ser and Pro amino acid residues and has a length of one or more amino acid residues or absent.

5. The hybrid peptide according to claims 1-3, characterized in that the hybrid peptide has a N-to C-terminal domain: (N-terminus)-chymotrypsin inhibitory peptide-insulin B-chain—insulin A-chain-(C-terminus), wherein the C-terminus of the chymotrypsin inhibitory peptide is directly fused to the N-terminus of the insulin B-chain or the analog thereof via the linker peptide, the “(N-terminus)-chymotrypsin inhibitory peptide-insulin B-chain” is a polypeptide formed by fusion of the chymotrypsin inhibitory peptide and the insulin B-chain and is selected from the group consisting of polypeptides with the following amino acid sequences: SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 and SEQ ID NO: 50, the A-chain is selected from the group consisting of polypeptides with the following amino acid sequences: SEQ ID NO: 29, SEQ ID NO: 31 and SEQ ID NO: 33.

6. The hybrid peptide according to claim 5, characterized in that a precursor protein of the hybrid peptide has a N- to C-terminal domain: (N-terminus)-chymotrypsin inhibitory peptide-insulin B-chain-C-polypeptide-insulin A-chain-(C-terminus), wherein the C-terminus of the chymotrypsin inhibitory peptide is directly fused to the N-terminus of the insulin B-chain or the analog thereof via the linker peptide; the “(N-terminus)-chymotrypsin inhibitory peptide-insulin B-chain” is a polypeptide formed by fusion of the chymotrypsin inhibitory peptide and the insulin B-chain and is selected from the group consisting of polypeptides with the following amino acid sequences: SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 and SEQ ID NO: 50; the A-chain is selected from the group consisting of polypeptides with the following amino acid sequences: SEQ ID NO: 29, SEQ ID NO: 31 and SEQ ID NO: 33; The C-polypeptide is selected from the group consisting of polypeptides with the following amino acid sequence: SEQ ID NO: 70 and SEQ ID NO: 71, wherein the precursor protein is treated by using protease KEX2 digestion, tobacco etch virus (TEV) protease digestion, and nickel ion-induced chemical cleavage, and/or treated with or without carboxypeptidase B and peptidylglycine α-amidating monooxygenase.

7. The hybrid peptide according to claim 6, characterized in that the precursor protein of the hybrid peptide is selected from the group consisting of polypeptides with the following amino acid sequences: SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173 and SEQ ID NO: 174.

8. The hybrid peptide according to claims 1-3, characterized in that the hybrid peptide has a N- to C-terminal domain: (N-terminus)-insulin B-chain-chymotrypsin inhibitory peptide—insulin A-chain-(C-terminus), wherein the N-terminus of the chymotrypsin inhibitory peptide is directly fused to the C-terminus of the insulin B-chain or the analog thereof via the linker peptide; the “(N-terminus)-insulin B-chain-chymotrypsin inhibitory peptide” is a polypeptide formed by fusion of the insulin B-chain and the chymotrypsin inhibitory peptide and is selected from the group consisting of polypeptides with the following amino acid sequences: SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58; the A-chain is selected from the group consisting of polypeptides with the following amino acid sequences: SEQ ID NO: 29, SEQ ID NO: 31 and SEQ ID NO: 33.

9. The hybrid peptide according to claim 8, characterized in that a precursor protein of the hybrid peptide has a N- to C-terminal domain: (N-terminus)-insulin B-chain-chymotrypsin inhibitory peptide-C-polypeptide-insulin A-chain-(C-terminus), wherein the N-terminus of the chymotrypsin inhibitory peptide is directly fused to the C-terminus of the insulin B-chain or the analog thereof via the linker peptide; the “(N-terminus)-insulin B-chain-chymotrypsin inhibitory peptide” is a polypeptide formed by fusion of the insulin B-chain and the chymotrypsin inhibitory peptide and is selected from the group consisting of polypeptides with the following amino acid sequences: SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58; the A-chain is selected from the group consisting of polypeptides with the following amino acid sequences: SEQ ID NO: 29, SEQ ID NO: 31, SEQ ID NO: 33; the C-polypeptide is selected from the group consisting of polypeptides with the following amino acid sequence: SEQ ID NO: 72 and SEQ ID NO: 73; wherein the precursor protein is treated by using protease KEX2 digestion, tobacco etch virus (TEV) protease digestion and nickel ion-induced chemical cleavage, and/or treated with or without carboxypeptidase B and peptidylglycine α-amidating monooxygenase.

10. The hybrid peptide according to claim 9, characterized in that the precursor protein of the hybrid peptide is selected from the group consisting of polypeptides with the following amino acid sequence: SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195 and SEQ ID NO: 196.

11. The hybrid peptide according to claims 1-3, characterized in that the hybrid peptide has a N- to C-terminal domain: (N-terminus)-insulin B-chain—chymotrypsin inhibitory peptide-insulin A-chain-(C-terminus), wherein the C-terminus of the chymotrypsin inhibitory peptide is directly fused to the N-terminus of the insulin A-chain or the analog thereof via the linker peptide, and the B-chain of the insulin or the analog thereof is selected from the group consisting of polypeptides with the following amino acid sequences: SEQ ID NO: 34, SEQ ID NO: 35, SEQ ID NO: 36, SEQ ID NO: 39 and SEQ ID NO: 40; the “chymotrypsin inhibitory peptide-insulin A-chain-(C-terminus)” is a polypeptide formed by fusion of the chymotrypsin inhibitory peptide and the insulin A-chain and is selected from the group consisting of polypeptides with the following amino acid sequences: SEQ ID NO: 59, SEQ ID NO: 60, SEQ ID NO: 61 and SEQ ID NO: 62.

12. The hybrid peptide according to claim 11, characterized in that a precursor protein of the hybrid peptide has a N- to C-terminal domain: (N-terminus)-insulin B-chain-C-polypeptide-chymotrypsin inhibitory peptide-insulin A-chain-(C-terminus), wherein the C-terminus of the chymotrypsin inhibitory peptide is directly fused to the N-terminus of the insulin A-chain or the analog thereof via the linker peptide, the B-chain of the insulin or the analog thereof is selected from the group consisting of polypeptides with the following amino acid sequences: SEQ ID NO: 34, SEQ ID NO: 35, SEQ ID NO: 36, SEQ ID NO: 39 and SEQ ID NO: 40; the “chymotrypsin inhibitory peptide-insulin A-chain-(C-terminus)” is a polypeptide formed by fusion of the chymotrypsin inhibitory peptide and the insulin A-chain and is selected from the group consisting of polypeptides with the following amino acid sequences: SEQ ID NO: 59, SEQ ID NO: 60, SEQ ID NO: 61 and SEQ ID NO: 62; the C-polypeptide is selected from the group consisting of polypeptides with the following amino acid sequence: SEQ ID NO: 70 and SEQ ID NO: 71; wherein the precursor protein is treated by using protease KEX2 digestion, tobacco etch virus (TEV) protease digestion and nickel ion-induced chemical cleavage, and/or treated with or without carboxypeptidase B and peptidylglycine α-amidating monooxygenase.

13. The hybrid peptide according to claim 12, characterized in that the precursor protein of the hybrid peptide is selected from the group consisting of polypeptides with the following amino acid sequence: SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217 and SEQ ID NO: 218.

14. The hybrid peptide according to claims 1-3, characterized in that the hybrid peptide has a N- to C-terminal domain: (N-terminus)-insulin B-chain—insulin A-chain-chymotrypsin inhibitory peptide-(C-terminus), wherein the N-terminus of the chymotrypsin inhibitory peptide is directly fused to the C-terminus of the insulin A-chain or the analog thereof via the linker peptide; the B-chain of the insulin or the analog thereof is selected from the group consisting of polypeptides with the following amino acid sequence: SEQ ID NO: 34, SEQ ID NO: 35, SEQ ID NO: 36, SEQ ID NO: 39, SEQ ID NO: 40; the “insulin A-chain-chymotrypsin inhibitory peptide-(C-terminus)” is a polypeptide formed by fusion of the insulin A-chain and the chymotrypsin inhibitory peptide and is selected from the group consisting of polypeptides with the following amino acid sequence: SEQ ID NO: 63 and SEQ ID NO: 64.

15. The hybrid peptide, according to claim 14, characterized in that a precursor protein of the hybrid peptide has a N- to C-terminal domain: (N-terminus)-insulin B-chain-C-polypeptide-insulin A-chain-chymotrypsin inhibitory peptide-(C-terminus), wherein the N-terminus of the chymotrypsin inhibitory peptide is directly fused to the C-terminus of the insulin A-chain or the analog thereof via the linker peptide; the B-chain of the insulin or the analog thereof is selected from the group consisting of polypeptides with the following amino acid sequence: SEQ ID NO: 34, SEQ ID NO: 35, SEQ ID NO: 36, SEQ ID NO: 39 and SEQ ID NO: 40; the “insulin A-chain-chymotrypsin inhibitory peptide-(C-terminus)” is a polypeptide formed by fusion of the insulin A-chain and the chymotrypsin inhibitory peptide and is selected from the group consisting of polypeptides with the following amino acid sequence: SEQ ID NO: 63 and SEQ ID NO: 64; the C-polypeptide is selected from the group consisting of polypeptides with the following amino acid sequence: SEQ ID NO: 70 and SEQ ID NO: 71; wherein the precursor protein is treated by using protease KEX2 digestion, tobacco etch virus (TEV) protease digestion and nickel ion-induced chemical cleavage, and/or treated with or without carboxypeptidase B and peptidylglycine a-amidating monooxygenase.

16. The hybrid peptide according to claim 15, characterized in that the precursor protein of the hybrid peptide is selected from the group consisting of polypeptides with the following amino acid sequence: SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237 and SEQ ID NO: 238.

17. The hybrid peptide according to claims 1-3, characterized in that the hybrid peptide has a N- to C-terminal domain: (N-terminus)-insulin B-chain—insulin A-chain-chymotrypsin inhibitory peptide-A-chain-(C-terminus), wherein the chymotrypsin inhibitory peptide directly forms a fusion peptide within the A-chain of the insulin or the analog thereof; the B-chain of the insulin or the analog is selected from the group consisting of polypeptides with the following amino acid sequences: SEQ ID NO: 34, SEQ ID NO: 35, SEQ ID NO: 36, SEQ ID NO: 39 and SEQ ID NO: 40; the “insulin A-chain-chymotrypsin inhibitory peptide-A-chain-(C-terminus)” is a polypeptide formed by fusion of the chymotrypsin inhibitory peptide within the A-chain of insulin and is selected from the group consisting of polypeptides with the following amino acid sequence: SEQ ID NO: 65, SEQ ID NO: 66, SEQ ID NO: 67 and SEQ ID NO: 68.

18. The hybrid peptide according to claim 17, characterized in that a precursor protein of the hybrid peptide has a N- to C-terminal domain: (N-terminus)-insulin B-chain-C-polypeptide-insulin A-chain-chymotrypsin inhibitory peptide-A-chain-(C-terminus), wherein the chymotrypsin inhibitory peptide directly forms a fusion peptide within the A-chain of the insulin or the analog thereof; the B-chain of the insulin or the analog is selected from the group consisting of polypeptides with the following amino acid sequences: SEQ ID NO: 34, SEQ ID NO: 35, SEQ ID NO: 36, SEQ ID NO: 39 and SEQ ID NO: 40; the “insulin A-chain chymotrypsin inhibitory peptide-A-chain-(C-terminus)” is a polypeptide formed by fusion of the chymotrypsin inhibitory peptide within the A-chain of the insulin and is selected from the group consisting of polypeptides with the following amino acid sequence: SEQ ID NO: 65, SEQ ID NO: 66, SEQ ID NO: 67 and SEQ ID NO: 68; the C-polypeptide is selected from the group consisting of polypeptides with the following amino acid sequence: SEQ ID NO: 70 and SEQ ID NO: 71; wherein the precursor protein is treated by using protease KEX2 digestion, tobacco etch virus (TEV) protease digestion and nickel ion-induced chemical cleavage, and/or treated with or without carboxypeptidase B and peptidylglycine a-amidating monooxygenase.

19. The hybrid peptide according to claim 18, characterized in that the precursor protein of the hybrid peptide is selected from the group consisting of polypeptides with the following amino acid sequences: SEQ ID NO: 249, SEQ ID NO: 250, SEQ ID NO: 251, SEQ ID NO: 252, SEQ ID NO:
253, SEQ ID NO: 254, SEQ ID NO: 255, SEQ ID NO: 256, SEQ ID NO: 257 and SEQ ID NO: 258.

20. A method for preparing the hybrid peptide comprising the chymotrypsin inhibitory peptide and the insulin or the analog thereof according to claims 1-19, characterized in that the N-terminus of the precursor protein contains a cleavage site of TEV protease as shown in SEQ ID NO: 69, and the linker peptide between the B-chain and the A-chain of the insulin molecule or the analog thereof is selected from the group consisting of the following polypeptide: SEQ ID NO: 70, SEQ ID NO:
71, SEQ ID NO: 72 and SEQ ID NO: 73; wherein the precursor protein is treated by using protease KEX2 digestion, TEV digestion and nickel ion-induced chemical cleavage, and/or treated with or without carboxypeptidase B and peptidylglycine α-amidating monooxygenase.

21. A use of the hybrid peptide according to claims 1-19 in the preparation of drugs for treating type I diabetes or type II diabetes.

22. The use according to claim 21, characterized in that the hybrid peptide can be administered by subcutaneous injection and orally when treating type I diabetes and type II diabetes.
